(19) United States
(12) Patent Application Publication
(10) Pub. No.: US 2014/0286980 A1
(43) Pub. Date: Sep. 25, 2014

(54) INFECTIOUS CLONES OF TORQUE TENO VIRUS

(71) Applicant: Jacqueline Gayle Marx, Portage, MI (US)

(72) Inventor: Jacqueline Gayle Marx, Portage, MI (US)

(73) Assignee: ZOETIS LLC, Florham Park, NJ (US)

(21) Appl. No.: 14/122,423

(22) PCT Filed: May 24, 2012
(86) PCT No.: PCT/IB2012/052628

§ 371 Date: May 15, 2014

Related U.S. Application Data
(60) Provisional application No. 61/494,674, filed on Jun. 8, 2011.

(51) Int. Cl. C12N 7/00

(52) U.S. Cl. CPC C12N 7/00
USPC 424/186.1; 536/23.72; 435/320.1; 435/254.2; 435/252.33; 435/235.1; 435/236

(57) ABSTRACT

The present invention is directed to novel nucleotide and amino acid sequences of Torque teno virus (“TTV”), including novel genotypes thereof, all of which are useful in the preparation of vaccines for treating and preventing diseases in swine and other animals. Vaccines provided according to the practice of the invention are effective against multiple swine TTV genotypes and isolates. Diagnostic and therapeutic polyclonal and monoclonal antibodies are also a feature of the present invention, as are infectious clones useful in the propagation of the virus and in the preparation of vaccines. Particularly important aspects of the invention include polynucleotide constructs that replicate in tissue culture and in host swine. The invention also provides for novel full length TTV genomes that can replicate efficiently in host animals and tissue culture.

### FIELD OF THE INVENTION

The present invention is directed to novel nucleotide and amino acid sequences of Torque teno virus (“TTV”), including novel genotypes thereof, all of which are useful in the preparation of vaccines for treating and preventing diseases in swine and other animals. Vaccines provided according to the practice of the invention are effective against multiple swine TTV genotypes and isolates. Diagnostic and therapeutic polyclonal and monoclonal antibodies are also a feature of the present invention, as are infectious clones useful in the propagation of the virus and in the preparation of vaccines. Of particular importance, there are disclosed vaccines that comprise, as antigen, the expressed protein of single TTV open reading frames, most particularly from ORF1 or ORF2, and also fragments of the full length ORF1 and ORF2-encoded proteins. The invention also provides for novel full length TTV genomes that can replicate efficiently in host animals and tissue culture.

### BACKGROUND OF THE INVENTION

Torque Teno Virus (“TTV”), also referred to as transfusion-transmitted virus, is generally assigned to the Circoviridae family. It is generally recognized that TTV was first isolated from human transfusion patients (see for example, Nishizawa et al., Biochem. Biophys. Res. Comm. vol. 241, 1997, pp. 92-97). Subsequently, TTV or TTV-like viruses have been identified from other mammals, including swine, and numerous strains or isolates have been published (see for example, McKeown et al. Vet. Microbiol. vol. 104, 2004, pp 113-117).

Subsequent work as shown that TTV and TTV-like viruses are very common; however the pathogenesis of TTV, and the contributions it may make to other disease states (for example, those caused by other viruses and bacteria) remains unclear. For example, TTV infections appear to be common in humans, including even in healthy individuals, and such infections are often asymptomatic, and may remain for years. In addition, the general inability to propagate the virus in cell culture, and a lack of any clear mechanistic disease models, have made any overall characterization of TTV biology difficult. Notwithstanding that TTV viremia is elevated in human patients afflicted with other viral diseases, (such as hepatitis or HIV/AIDS), there is also considerable medical literature suggesting that TTVs are, in fact, avirulent, and await any clear actual association with known disease states. See, for example, Biagini et al., Vet. Microbiol. vol. 98, 2004, pp. 95-101.

In regard of swine, the situation is similar. There is considerable work suggesting that TTV infection is associated with, and contributes to, numerous diseases such as porcine circovirus disease (and its various clinical manefestations, such as postweaning multisystemic wasting syndrome and respiratory disease complicated by lung lesions), and PRRSV-associated disease (porcine respiratory and reproductive syndrome virus). See for example published international patent applications WO 2008/150275 and WO 2008/127279. Krakowka et al. also report on an often fatal disease in pigs referred to as PDNS (porcine dermatitis and neuropathy syndrome) which is described as a manifestation of disseminated intravascular coagulation, and for which combined infection by serotype 1 TTV and PRRSV virus was possibly implicated (Am. J. Vet Res, vol 69(12), 2008, pp. 1615-1622. PDNS disease was also correlated with porcine circovirus disease (notably PCV-2) and also with bacterial infections. Accordingly, while considerable work has been accomplished, there remains little work that definitively correlates porcine TTV infection with specific pathologies. Nonetheless, it has become reasonably clear that TTV infection can potentiate numerous disease states. Accordingly, there is a need for various classes of TTV reagents, such as high affinity antibodies, and for example, peptide fragments of TTV or whole virions that are highly immunizing, both to further our understanding of overall TTV biology and to vaccinate, directly or indirectly, against numerous disease states to which TTV may contribute.

Thus, although the possibility exists that TTV is the principle causative factor of diseases in swine, it seems more likely that numerous swine diseases either require the presence of more than one virus, or that the primary effect of certain “primary” pathogens is potentiated by TTV infection. As stated, the possibility exists that numerous diseases of swine can be treated or lessened by administering anti-TTV agents to affected or potentially affected animals. Notwithstanding the well established interest in TTV, effective vaccines have not emerged.

TTV is a small, non-enveloped virus comprised of negative polarity, single-strand circularized DNA. The genome includes three major open reading frames, ORF1, ORF2 and ORF3, which overlap, and ORF1 encodes the capsid protein. (Biagini et al., supra). For a detailed discussion thereof, please see the following references, which are incorporated by referenece: Kakkola et al., Virology, vol. 382 (2008), pp. 182-189; Mushahwar et al., Proc. Natl. Acad. Sci, USA, vol 96, (1999) pp. 3177-3182; and T. Kekarainen and J. Segales, “Torque teno virus infection in the pig and its potential role as a model of human infection”, The Veterinary Journal, accepted Dec. 13, 2007 for 2008.

Despite the relatively simple genome, it has been generally very difficult to propagate the virus in cell culture or by other in vitro methods. The present invention is directed to recombinant constructs whereby TTV can be propagated in vitro, including via infectious clones. More particularly, the invention is directed to the discovery that effective vaccines can in fact be made from TTV, most particularly when the TTV antigen is the expression product of a single ORF, or a fragment thereof. In a preferred embodiment, the invention provides for ORFI protein vaccines.

### SUMMARY OF THE INVENTION

The present invention provides a method of treating or preventing a disease or disorder in an animal caused by infection with torque teno virus (TTV), including disease states that are directly caused by TTV, and disease states contributed to or potentiated by TTV. In a preferred example, the animal treated is a swine. Disease states in swine that may be potentiated by TTV, and which may also be treated or prevented according to the practice of the invention, include those caused by or associated with porcine circovirus (PCV), and porcine reproductive and respiratory syndrome virus (PRRS).

The present invention also includes the option to administer a combination vaccine, that is, a bivalent or multivalent combination of antigens, which may include live, modified live, or inactivated antigens against the non-TTV pathogen, with appropriate choice of adjuvant.

Based in part upon the unique TTV amino acid sequences as disclosed herein, the present invention also provides a diagnostic kit for differentiating between porcine animals vaccinated with the above described TTV vaccines and porcine animals infected with field strains of TTV.

Representative embodiments of the invention include an isolated polynucleotide sequence that comprises a polynucleotide selected from the group consisting of:

(a₁) the DNA of genotype 2 sequence TTV13 (SEQ ID NO: 1); the DNA genotype 2 sequence TTV10 (SEQ ID NO: 2); or a fragment thereof than encodes the TTV capsid protein or a fragment of said protein;

(a₂) the DNA of a genotype 1 sequence selected from the group consisting of ttvg1-7 (SEQ ID NO: 4), ttvGT1-17 (SEQ ID NO: 5), ttvGT1-21 (SEQ ID NO: 6), ttvgt1-27 (SEQ ID NO: 3), ttvgt1-178 (SEQ ID NO: 7) or a fragment thereof than encodes the TTV capsid protein or a fragment of said protein;

(b) the complement of any sequence in (a);

(c) a polynucleotide that hybridizes with a sequence of (a) or (b) under stringent conditions defined as hybriding to filter bound DNA in 0.5M NaHPO₄, 7% SDS, 1 mM EDTA at 65° C., and washing in 0.1×SSC/0.1% SDS at 68° C.;

(d) a polynucleotide that is at least 70% identical to the polynucleotide of (a) or (b);

(e) a polynucleotide that is at least 80% identical to the polynucleotide of (a) or (b);

(f) a polynucleotide that is at least 90% identical to the polynucleotide of (a) or (b); and

(g) a polynucleotide that is at least 95% identical to the polynucleotide of (a) or (b).

The invention further provides RNA polynucleotide molecules that are the complement of any such DNA polynucleotide sequence, and vectors and plasmids for the expression of any such RNA or DNA polynucleotides, and for TTV virus that is expressed from such nucleotide sequences, wherein said virus is live, or fully or partially attenuated.

The invention also provides a DNA vaccine that comprises a polynucleotide sequence as aforementioned, and corresponding nucleotide sequences that function as infectious clones.

The invention provides a polypeptide encoded by any of the open reading frames of the genotype 2 TTV13 (SEQ ID NO:1) or genotype 2 TTV10 (SEQ ID NO: 2) polynucleotides, or a polypeptide that is at least 90% identical thereto, or to a fragment thereof, including the option that additional otherwise identical amino acids are replaced by conservative substitutions.

The invention also provides a polypeptide encoded by any of the open reading frames of the (all serotype 1) ttvg1-7 (SEQ ID NO:10), ttvGT1-17 (SEQ ID NO:11), ttvGT1-21 (SEQ ID NO:12), ttvgt1-27 (SEQ ID NO:13), and ttvgt1-178 (SEQ ID NO:9) ORF1 polynucleotides, or a polypeptide that is at least 90% identical thereto, or to a fragment thereof, including the option that additional otherwise identical amino acids are replaced by conservative substitutions.

Despite continued failures as reported in the art, to provide effective vaccines against TTV (or to limit the ability of TTV to potentiate other diseases), the present invention provides for such effective vaccines, which preferably comprise a polypeptide resultant from expression of a single TTV open reading frame, or a mixture thereof. In a preferred embodiment, the polypeptide is expressed from ORF1, and preferred mixtures include a combination of the polypeptides of ORF1 and ORF2, and ORF1 and ORF3.

In a further preferred embodiment, and taking advantage of the substantial polypeptide sequence information disclosed herein, there are further provided polypeptide vaccines wherein the antigen is defined by (a) the first 100 N-terminal amino acids of the capsid protein of TTV13 (SEQ NO:1) or TTV10 (SEQ ID NO:2); or (b) an amino acid sequence that is at least 90 percent identical thereto; or (c) an arginine rich region thereof.

The invention also provides for novel full length TTV genomes that can replicate efficiently in host animals and tissue culture.

### BRIEF DESCRIPTION OF THE DRAWINGS

FIG. 1 (panels A and B) shows detection of ORF1 protein by immunological methods.

FIG. 2 evidences successful expression of codon-optimized TTVg1 ORF1 protein in E. coli, with a 6X His tag for affinity purification.

FIG. 3 provides a vector map for the Chromos construct pcTV-TTV1-7 ORF1 (plus yeast invertase) expression plasmid from which is expressed (following integration into an artificial chromosome in CHO cells) vaccinating ORF1 protein.

FIG. 4 provides a phylogenetic tree for various TTV strains including a compilation of percent identities.

FIG. 5 (panels A, B and C) provides identification of in-common arginine rich regions of ORF1 proteins as expressed from various TTV isolates.

FIG. 6 provides a vector map for TTVg1-178 as assembled.

FIG. 7 demonstrates that Chromos-expressed g1TTV ORF1 significantly reduced lung lesions compared to the challenge controls and reduces the magitude and duration of g1TTV viremia, again compared to the challenge controls.

FIG. 8 provides a vector map for the pCR2.1+TTVg1-178 construct that contains a ttvg1-178 strain full length infectious clone.

FIG. 9 provides a vector map for the pCR2.1+TTVg1-178 construct that contains a ttvg1-178 strain full length infectious clone with a 218 bp partial duplication.

FIG. 10 provides a vector map for the pCR2.1+TTVg1-178 construct that contains a ttvg1-178 strain full length infectious clone with a 518 bp partial duplication.

FIG. 11 provides the nucleotide sequence for the construct of FIG. 9.

FIG. 12 provides the nucleotide sequence for the construct of FIG. 10.

### BRIEF DESCRIPTION OF THE SEQUENCE LISTINGS

SEQ ID NO:1 provides the genotype gt2 TTV 10 DNA sequence.

SEQ ID NO:2 provides the genotype 2 gt2 TTV 13 DNA sequence.

SEQ ID NO:3 provides the genotype 1 ttvgt1-27 DNA sequence.

SEQ ID NO:4 provides the genotype 1 ttvgt1-7 DNA sequence.

SEQ ID NO:5 provides the genotype 1 ttvgt1-17 DNA sequence.

SEQ ID NO:6 provides the genotype 1 ttvgt1-21 DNA sequence.

SEQ ID NO:7 provides the genotype 1 ttvg1-178 DNA sequence

SEQ ID NO:8 provides the amino acid sequence of TTV strain AY823991 ORF1.

SEQ ID NO:9 provides the amino acid sequence of TTV strain ttvgt1-178 ORF1 (TTV genotype 1).

SEQ ID NO:10 provides the amino acid sequence of TTV strain ttvgt1-7 ORF1.

SEQ ID NO:11 provides the amino acid sequence of TTV strain ttvgt1-17 ORF1.

SEQ ID NO:12 provides the amino acid sequence of TTV strain ttvgt1-21 ORF1.

SEQ ID NO:13 provides the amino acid sequence of TTV strain ttvgt1-27 ORF1.

SEQ ID NO:14 provides the amino acid sequence of TTV strain gt2 TTV10 ORF1 (genotype 2).

SEQ ID NO:15 provides the amino acid sequence of TTV strain gt2 TTV13 ORF1.

SEQ ID NO:16 provides the DNA sequence of known strain AY823991 (genotype 2).

SEQ ID NO:17 provides the DNA sequence of known strain AY823990 (genotype 1).

SEQ ID NO:18 provides the 76057-3 TTV capsid encoding sequence, codon optimized for E. coli. as cloned into the pUC57 GenScript® vector.

SEQ ID NO:19 provides the 76057-4 TTV capsid encoding sequence, codon optimized for E. coli. as cloned into the Invitrogen pET101/D-TOPO® expression plasmid.

SEQ ID NO:20 provides the 76057-5 TTV capsid encoding sequence, codon optimized for Saccharomyces cerevisiae as cloned into the pUC57 GenScript® vector.

SEQ ID NO:21 provides the DNA sequence for a construct that encodes ttvgt1-7 ORF1 with a yeast invertase expression tag (YI).

SEQ ID NO:22 provides a ttvgt1 peptide sequence (numbering based on the corresponding AY823990 sequence) from the ORF1 capsid protein corresponding to residues 167-185, which is used with the C-terminal AA in amidated form.

SEQ ID NO:23 provides a ttvgt1 peptide sequence (numbering based on the corresponding AY823990 sequence) from the ORF1 capsid protein corresponding to residues 459-479.

SEQ ID NO:24 provides a ttvgt1 peptide sequence (numbering based on the corresponding AY823990 sequence) from the ORF1 capsid protein corresponding to residues 612-637.

SEQ ID NO:25 provides the amino acid sequence of TTV strain AY823990 ORF1.

SEQ ID NOS:26-29 define primer sequences.

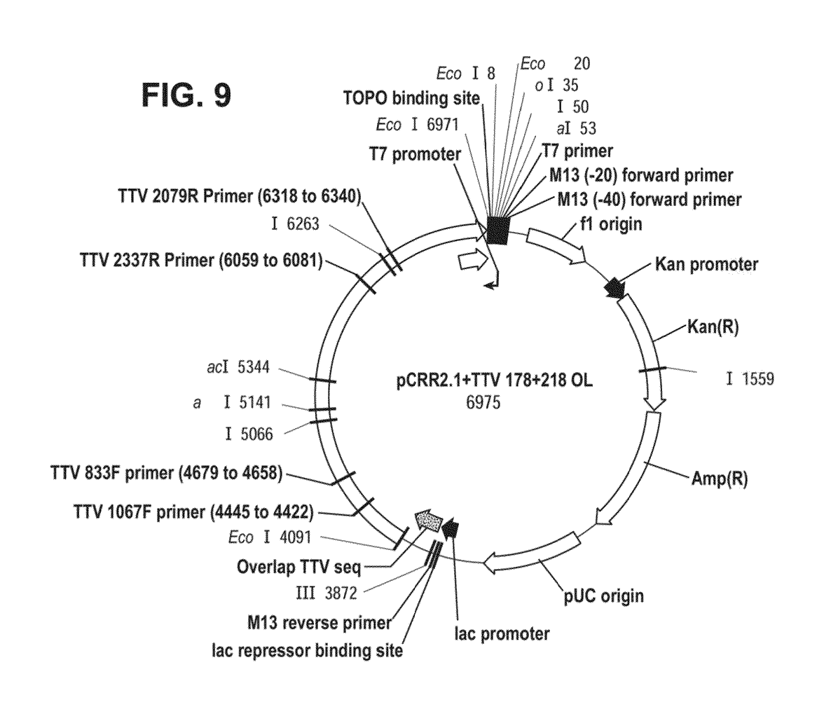

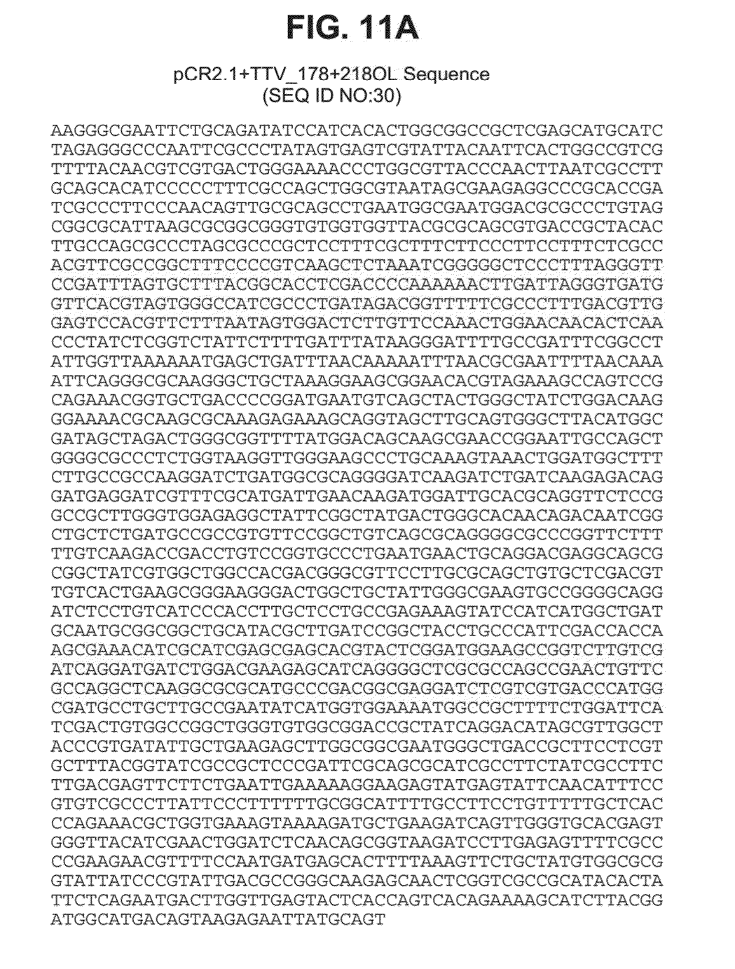

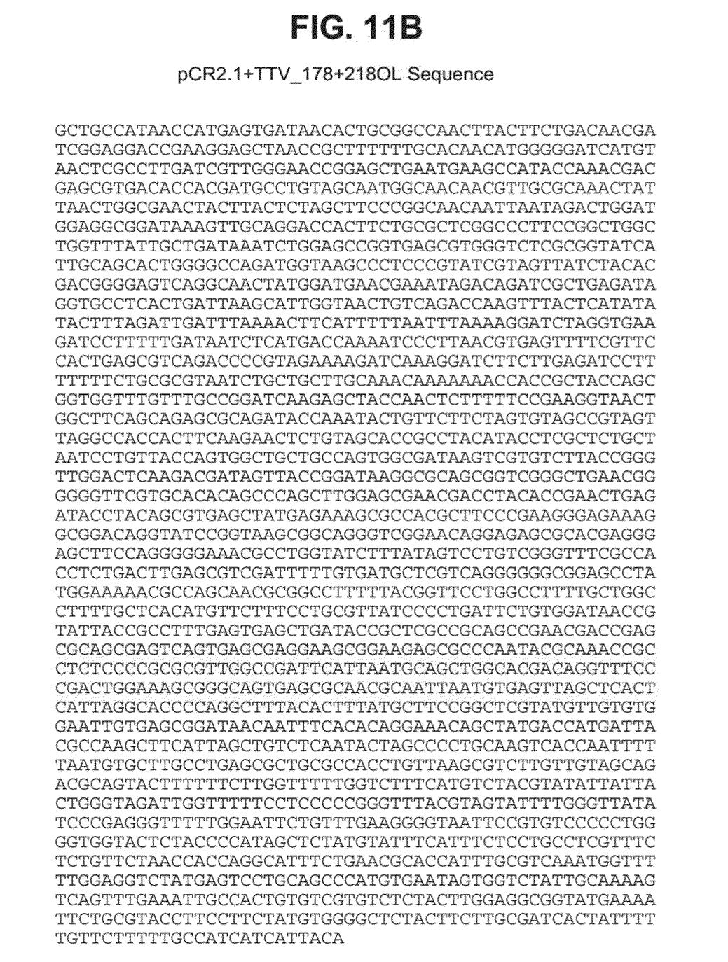

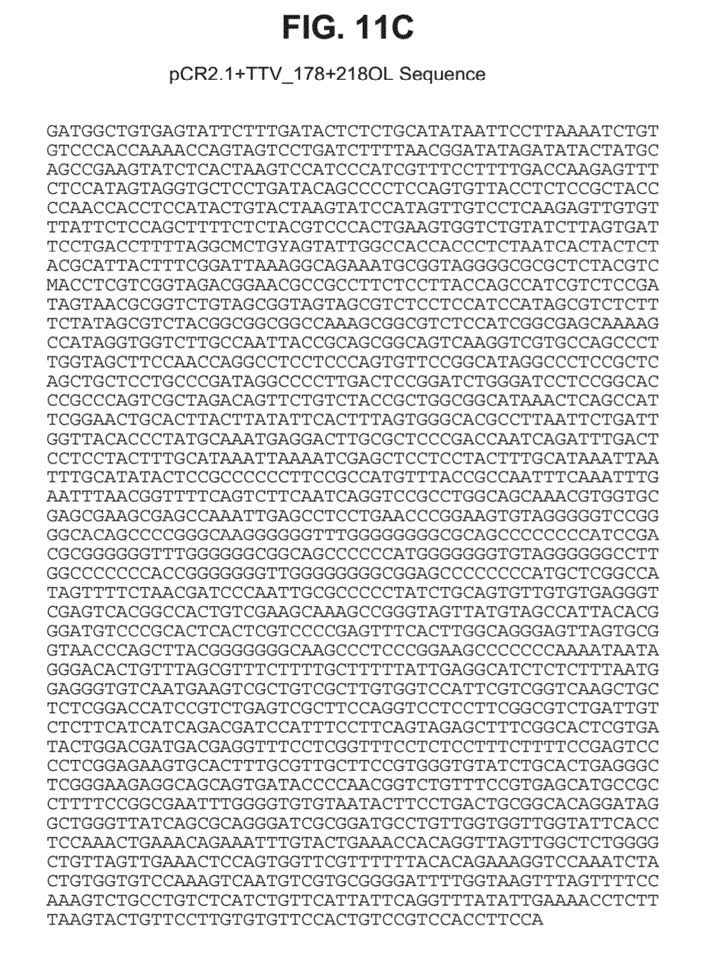

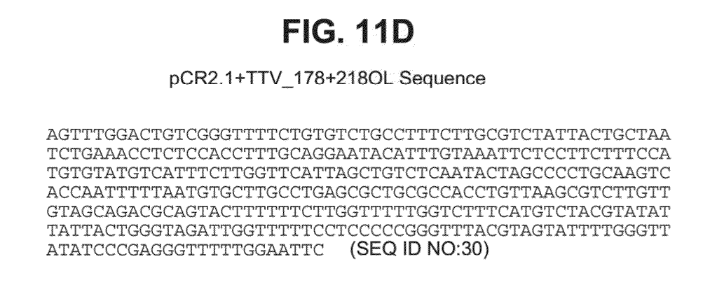

SEQ ID NO: 30 provides the nucleotide sequence of the pCR2.1+TTV-178 clone having an additional +218 nucleotides, partial duplication (see FIGS. 9, 11).

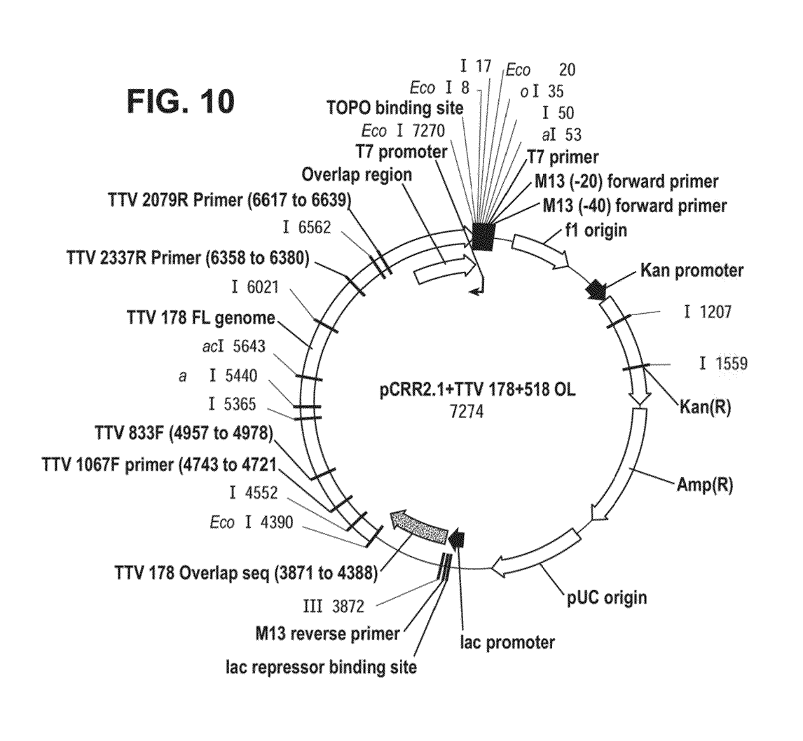

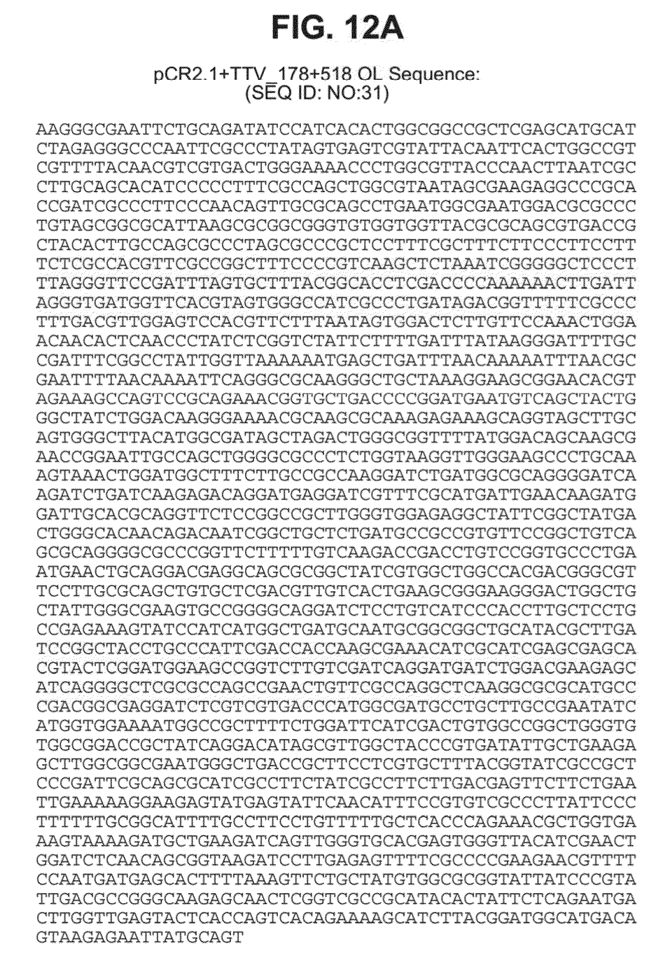

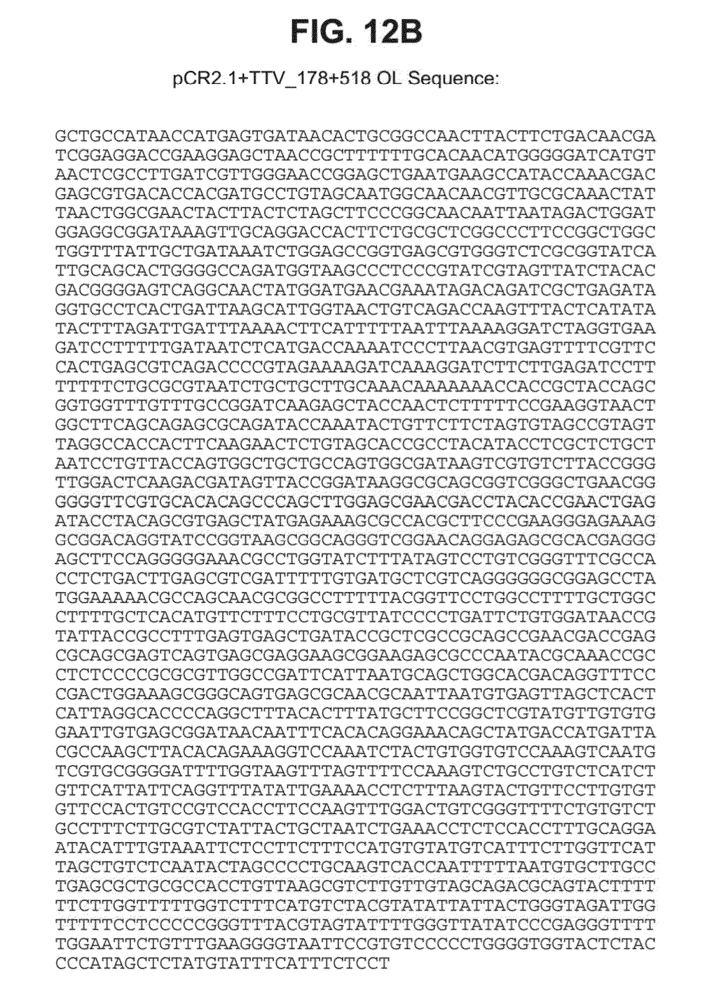

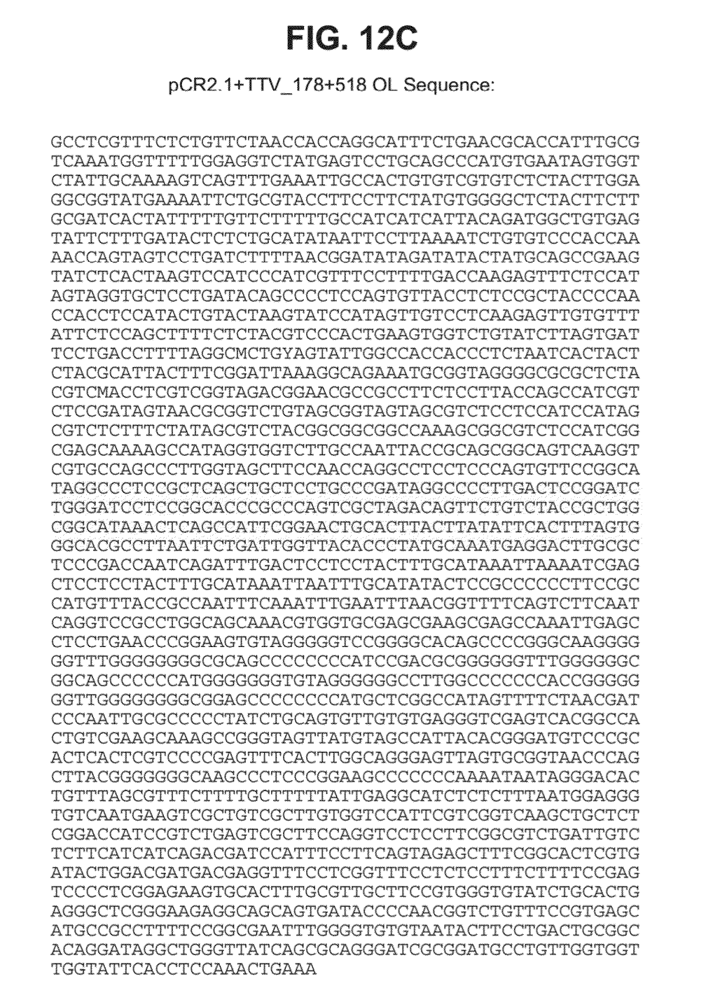

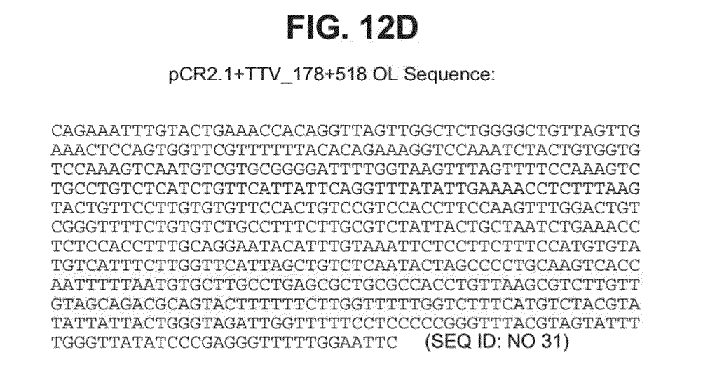

SEQ ID NO: 31 provides the nucleotide sequence of the pCR2.1+TTV-178 clone having an additional +518 nucleotides, partial duplication. (see FIGS. 10, 12).

SEQ ID NOS: 32-34 define primer sequences.

In connection with the descriptors for the sequences, those familiar with the art will recognize that numerous slightly different abbreviations are commonly used interchangeably for specific serotypes, for example, g1TTV, TTVg1, genotype 1 TTV, serotype 1 TTV, gt1TTV, and the like. A similar situation exists for genotype 2.

### DETAILED DESCRIPTION OF THE INVENTION

The following definitions and introductory matters are applicable in the specification.

The terms “porcine” and “swine” are used interchangeably herein and refer to any animal that is a member of the family Suidae such as, for example, a pig. “Mammals” include any warm-blooded vertebrates of the Mammalia class, including humans.

An “infectious DNA molecule”, for purposes of the present invention, is a DNA molecule that encodes the necessary elements for viral replication, transcription, and translation into a functional virion in a suitable host cell.

Likewise, an “isolated polynucleotide molecule” refers to a composition of matter comprising a polynucleotide molecule of the present invention purified to any detectable degree from its naturally occurring state, if any.

For purposes of the present invention, the nucleotide sequence of a second polynucleotide molecule (either RNA or DNA) is “homologous” to the nucleotide sequence of a first polynucleotide molecule, or has “identity” to said first polynucleotide molecule, where the nucleotide sequence of the second polynucleotide molecule encodes the same polyaminoacid as the nucleotide sequence of the first polynucleotide molecule as based on the degeneracy of the genetic code, or when it encodes a polyaminoacid that is sufficiently similar to the polyaminoacid encoded by the nucleotide sequence of the first polynucleotide molecule so as to be useful in practicing the present invention. Homologous polynucleotide sequences also refers to sense and anti-sense strands, and in all cases to the complement of any such strands. For purposes of the present invention, a polynucleotide molecule is useful in practicing the present invention, and is therefore homologous or has identity, where it can be used as a diagnostic probe to detect the presence of TTV virus or viral polynucleotide in a fluid or tissue sample of an infected pig, e.g. by standard hybridization or amplification techniques. Generally, the nucleotide sequence of a second polynucleotide molecule is homologous to the nucleotide sequence of a first polynucleotide molecule if it has at least about 70% nucleotide sequence identity to the nucleotide sequence of the first polynucleotide molecule as based on the BLASTN algorithm (National Center for Biotechnology Information, otherwise known as NCBI, (Bethesda, Md., USA) of the United States National Institute of Health). In a specific example for calculations according to the practice of the present invention, reference is made to BLASTP 2.2.6 [Tatusova TA and TL Madden, “BLAST 2 sequences—a new tool for comparing protein and nucleotide sequences.” (1999) FEMS Microbiol Lett. 174:247-250.]. Briefly, two amino acid sequences are aligned to optimize the alignment scores using a gap opening penalty of 10, a gap extension penalty of 0.1, and the “blosum62” scoring matrix of Henikoff and Henikoff (Proc. Nat. Acad. Sci. USA 325 89:10915-10919. 1992). The percent identity is then calculated as: Total number of identical matches×100/divided by the length of the longer sequence+number of gaps introduced into the longer sequence to align the two sequences.

Preferably, a homologous nucleotide sequence has at least about 75% nucleotide sequence identity, even more preferably at least about 80%, 85%, 90% and 95% nucleotide sequence identity. Since the genetic code is degenerate, a homologous nucleotide sequence can include any number of “silent” base changes, i.e. nucleotide substitutions that nonetheless encode the same amino acid.

A homologous nucleotide sequence can further contain non-silent mutations, i.e. base substitutions, deletions, or additions resulting in amino acid differences in the encoded polyaminoacid, so long as the sequence remains at least about 70% identical to the polyaminoacid encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tyrptophan and phenylalanine.

Homologous nucleotide sequences can be determined by comparison of nucleotide sequences, for example by using BLASTN, above. Alternatively, homologous nucleotide sequences can be determined by hybridization under selected conditions. For example, the nucleotide sequence of a second polynucleotide molecule is homologous to SEQ ID NO:1 (or any other particular polynucleotide sequence) if it hybridizes to the complement of SEQ ID NO:1 under moderately stringent conditions, e.g., hybridization to filter-bound DNA in 0.5 M NaHPO₄, 7% sodium dodecyl sulfate (SDS), 1 mM EDTA at 65° C., and washing in 0.2×SSC/0.1% SDS at 42° C. (see Ausubel et al editors, Protocols in Molecular Biology, Wiley and Sons, 1994, pp. 6.0.3 to 6.4.10), or conditions which will otherwise result in hybridization of sequences that encode a TTV virus as defined below. Modifications in hybridization conditions can be empirically determined or precisely calculated based on the length and percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

In another embodiment, a second nucleotide sequence is homologous to SEQ ID NO:1 (or any other sequence of the invention) if it hybridizes to the complement of SEQ ID NO:1 under highly stringent conditions, e.g. hybridization to filter-bound DNA in 0.5 M NaHPO₄, 7% SDS, 1 mM EDTA at 65° C., and washing in 0.1×SSC/0.1% SDS at 68° C., as is known in the art.

It is furthermore to be understood that the isolated polynucleotide molecules and the isolated RNA molecules of the present invention include both synthetic molecules and molecules obtained through recombinant techniques, such as by in vitro cloning and transcription.

### Polypeptides and Polynucleotides of the Invention

Representative embodiments of the invention include an isolated polynucleotide sequence that comprises a polynucleotide selected from the group consisting of:

(a₁) the DNA of genotype 2 sequence TTV13 (SEQ ID NO: 1); the DNA genotype 2 sequence TTV10 (SEQ ID NO: 2); or a fragment thereof than encodes the TTV capsid protein or a fragment of said protein;

(a₂) the DNA of a genotype 1 sequence selected from the group consisting of ttvg1-7 (SEQ ID NO: 4), ttvGT1-17 (SEQ ID NO: 5), ttvGT1-21 (SEQ ID NO: 6), ttvgt1-27 (SEQ ID NO: 3), ttvgt1-178 (SEQ ID NO: 7) or a fragment thereof than encodes the TTV capsid protein or a fragment of said protein;

(b) the complement of any sequence in (a);

(c) a polynucleotide that hybridizes with a sequence of (a) or (b) under stringent conditions defined as hybriding to filter bound DNA in 0.5M NaHPO₄, 7% SDS, 1 mM EDTA at 65° C., and washing in 0.1×SSC/0.1% SDS at 68° C.;

(d) a polynucleotide that is at least 70% identical to the polynucleotide of (a) or (b);

(e) a polynucleotide that is at least 80% identical to the polynucleotide of (a) or (b);

(f) a polynucleotide that is at least 90% identical to the polynucleotide of (a) or (b); and

(g) a polynucleotide that is at least 95% identical to the polynucleotide of (a) or (b).

The invention also provides a polypeptide encoded by any of the open reading frames of the genotype 2 TTV13 (SEQ ID NO:1) or genotype 2 TTV10 (SEQ ID NO:2) polynucleotides, or a polypeptide that is at least 90% identical thereto, or to a fragment thereof, including the option that additional otherwise identical amino acids are replaced by conservative substitutions.

The invention also provides a polypeptide encoded by any of the open reading frames of the (all serotype 1) ttvg1-7 (SEQ ID NO:10), ttvGT1-17 (SEQ ID NO:11), ttvGT1-21 (SEQ ID NO:12), ttvgt1-27 (SEQ ID NO:13), and ttvgt1-178 (SEQ ID NO:9) ORF1 polynucleotides, or a polypeptide that is at least 90% identical thereto, or to a fragment thereof, including the option that additional otherwise identical amino acids are replaced by conservative substitutions.

In a preferred embodiment, the polypeptide is expressed from ORF1, and preferred mixtures include a combination of the polypeptides of ORF1 and ORF2, and ORF1 and ORF3.

In a further preferred embodiment, there are further provided TTV polypeptide-based vaccines wherein the antigen is defined by:

(a) the first 300 N-terminal amino acids of the ORF1 capsid protein of TTV13 (SEQ NO:1) or TTV10 (SEQ ID NO:2); or (b) an amino acid sequence that is at least 90 percent identical thereto;
(b) the first 200 N-terminal amino acids of the ORF1 capsid protein of TTV13 (SEQ NO:1) or TTV10 (SEQ ID NO:2); or (b) an amino acid sequence that is at least 90 percent identical thereto;
(c) the first 100 N-terminal amino acids of the ORF1 capsid protein of TTV13 (SEQ NO:1) or TTV10 (SEQ ID NO:2); or (b) an amino acid sequence that is at least 90 percent identical thereto;
(d) the first 300 N-terminal amino acids of the ORF1 capsid protein of any of (all serotype 1) ttvg1-7 (SEQ ID NO:10), ttvGT1-17 (SEQ ID NO:11), ttvGT1-21 (SEQ ID NO:12), ttvgt1-27 (SEQ ID NO:13), and ttvgt1-178 (SEQ ID NO:9) or a polypeptide that is at least 90% identical thereto;
(e) the first 200 N-terminal amino acids of the ORF1 capsid protein of any of (all serotype 1) ttvg1-7 (SEQ ID NO:10), ttvGT1-17 (SEQ ID NO:11), ttvGT1-21 (SEQ ID NO:12), ttvgt1-27 (SEQ ID NO:13), and ttvgt1-178 (SEQ ID NO:9) or a polypeptide that is at least 90% identical thereto; and
(f) the first 100 N-terminal amino acids of the ORF1 capsid protein of any of (all serotype 1) ttvg1-7 (SEQ ID NO:10), ttvGT1-17 (SEQ ID NO:11), ttvGT1-21 (SEQ ID NO:12), ttvgt1-27 (SEQ ID NO:13), and ttvgt1-178 (SEQ ID NO:9) or a polypeptide that is at least 90% identical thereto.

### Further Genetic Manipulations

The DNA and amino acid sequence information provided by the present invention also makes possible the systematic analysis of the structure and function of the viral genes and their encoded gene products. Knowledge of a polynucleotide encoding a viral gene product of the invention also makes available anti-sense polynucleotides which recognize and hybridize to polynucleotides encoding a polypeptide of the invention, or a fragment thereof. Full length and fragment anti-sense polynucleotides are useful in this respect. The worker of ordinary skill will appreciate that fragment anti-sense molecules of the invention include (i) those which specifically recognize and hybridize to a specific RNA (as determined by sequence comparison of DNA encoding a viral polypeptide of the invention as well as (ii) those which recognize and hybridize to RNA encoding variants of the encoded proteins. Antisense polynucleotides that hybridize to RNA/DNA encoding other TTV peptides are also identifiable through sequence comparison to identify characteristic, or signature sequences for the family of molecules. Such techniques (see Example 8) are further of use in the study of antigenic domains in TTV polypeptides, and may also be used to distinguish between infection of a host animal with remotely related non-TTV members of the Circoviridae.

Example 4 provides guidance as to effective codon optimization for enhanced expression in yeast and E. coli for the constructs of the invention.

### Vaccine Formulations

Vaccines of the present invention can be formulated following accepted convention to include acceptable carriers for animals, including humans (if applicable), such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

Vaccines of the present invention may further comprise one or more additional immunomodulatory components such as, e.g., an adjuvant or cytokine, among others. Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines.

Additional adjuvant systems permit for the combination of both T-helper and B-cell epitopes, resulting in one or more types of covalent T-B epitope linked structures, with may be additionally lipidated, such as those described in WO2006/084319, WO2004/014957, and WO2004/014956.

In a preferred embodiment of the present invention, ORFI TTV protein, or other TTV proteins or fragments thereof, is formulated with 5% AMPHIGEN®.

Vaccines of the present invention can optionally be formulated for sustained release of the virus, infectious DNA molecule, plasmid, or viral vector of the present invention. Examples of such sustained release formulations include virus, infectious DNA molecule, plasmid, or viral vector in combination with composites of biocompatible polymers, such as, e.g., poly(lactic acid), poly(lactic-co-glycolic acid), methylcellulose, hyaluronic acid, collagen and the like. The structure, selection and use of degradable polymers in drug delivery vehicles have been reviewed in several publications, including A. Domb et al., 1992, Polymers for Advanced Technologies 3: 279-292, which is incorporated herein by reference. Additional guidance in selecting and using polymers in pharmaceutical formulations can be found in texts known in the art, for example M. Chasin and R. Langer (eds), 1990, “Biodegradable Polymers as Drug Delivery Systems” in: Drugs and the Pharmaceutical Sciences, Vol. 45, M. Dekker, NY, which is also incorporated herein by reference. Alternatively, or additionally, the virus, plasmid, or viral vector can be microencapsulated to improve administration and efficacy. Methods for microencapsulating antigens are well-known in the art, and include techniques described, e.g., in U.S. Pat. No. 3,137,631; U.S. Pat. No. 3,959,457; U.S. Pat. No. 4,205,060; U.S. Pat. No. 4,606,940; U.S. Pat. No. 4,744,933; U.S. Pat. No. 5,132,117; and International Patent Publication WO 95/28227, all of which are incorporated herein by reference.

Liposomes can also be used to provide for the sustained release of virus, plasmid, viral protein, or viral vector. Details concerning how to make and use liposomal formulations can be found in, among other places, U.S. Pat. No. 4,016,100; U.S. Pat. No. 4,452,747; U.S. Pat. No. 4,921,706; U.S. Pat. No. 4,927,637; U.S. Pat. No. 4,944,948; U.S. Pat. No. 5,008,050; and U.S. Pat. No. 5,009,956, all of which are incorporated herein by reference.

An effective amount of any of the above-described vaccines can be determined by conventional means, starting with a low dose of virus, viral protein plasmid or viral vector, and then increasing the dosage while monitoring the effects. An effective amount may be obtained after a single administration of a vaccine or after multiple administrations of a vaccine. Known factors can be taken into consideration when determining an optimal dose per animal. These include the species, size, age and general condition of the animal, the presence of other drugs in the animal, and the like. The actual dosage is preferably chosen after consideration of the results from other animal studies (see, for example, Examples 2 and 3 below).

One method of detecting whether an adequate immune response has been achieved is to determine seroconversion and antibody titer in the animal after vaccination. The timing of vaccination and the number of boosters, if any, will preferably be determined by a doctor or veterinarian based on analysis of all relevant factors, some of which are described above.

The effective dose amount of virus, protein, infectious DNA molecule, plasmid, or viral vector, of the present invention can be determined using known techniques, taking into account factors that can be determined by one of ordinary skill in the art such as the weight of the animal to be vaccinated. The dose amount of virus of the present invention in a vaccine of the present invention preferably ranges from about 10¹ to about 10⁹ pfu (plaque forming units), more preferably from about 10² to about 10⁸ pfu, and most preferably from about 10³ to about 10⁷ pfu. The dose amount of a plasmid of the present invention in a vaccine of the present invention preferably ranges from about 0.1 μg to about 100 mg, more preferably from about 1 μg to about 10 mg, even more preferably from about 10 μg to about 1 mg. The dose amount of an infectious DNA molecule of the present invention in a vaccine of the present invention preferably ranges from about 0.1 μg to about 100 mg, more preferably from about 1 μg to about 10 mg, even more preferably from about 10 μg to about 1 mg. The dose amount of a viral vector of the present invention in a vaccine of the present invention preferably ranges from about 10¹ pfu to about 10⁹ pfu, more preferably from about 10² pfu to about 10⁸ pfu, and even more preferably from about 10³ to about 10⁷ pfu. A suitable dosage size ranges from about 0.5 ml to about 10 ml, and more preferably from about 1 ml to about 5 ml.

Suitable doses for viral protein or peptide vaccines according to the practice of the present invention range generally from 1 to 50 micrograms per dose, or higher amounts as may be determined by standard methods, with the amount of adjuvant to be determined by recognized methods in regard of each such substance. In a preferred example of the invention relating to vaccination of swine, an optimum age target for the animals is between about 1 and 21 days, which at pre-weening, may also correspond with other scheduled vaccinations such as against Mycoplasma hyopneumoniae. Additionally, a preferred schedule of vaccination for breeding sows would include similar doses, with an annual revaccination schedule.

### Antibodies

Also contemplated by the present invention are anti-TTV antibodies (e.g., monoclonal and polyclonal antibodies, single chain antibodies, chimeric antibodies, humanized, human, porcine, and CDR-grafted antibodies, including compounds which include CDR sequences which specifically recognize a TTV polypeptide of the invention. The term “specific for” indicates that the variable regions of the antibodies of the invention recognize and bind a TTV polypeptide exclusively (i.e., are able to distinguish a single TTV polypeptide from related polypeptides despite sequence identity, homology, or similarity found in the family of polypeptides), and which are permitted (optionally) to interact with other proteins (for example, S. aureus protein A or other antibodies in ELISA techniques) through interactions with sequences outside the variable region of the antibodies, and in particular, in the constant region of the Ab molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the TTV polypeptides of the invention are also contemplated, provided that the antibodies are first and foremost specific for, as defined above, a TTV polypeptide of the invention from which the fragment was derived.

For the purposes of clarity, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, as, Fv, Fab′, F(ab′)₂, as well as in single chains, and include synthetic polypeptides that contain all or part of one or more antibody single chain polypeptide sequences.

### Diagnostic Kits

The present invention also provides diagnostic kits. The kit can be valuable for differentiating between porcine animals naturally infected with a field strain of a TTV virus and porcine animals vaccinated with any of the TTV vaccines described herein. The kits can also be of value because animals potentially infected with field strains of TTV virus can be detected prior to the existence of clinical symptoms and removed from the herd, or kept in isolation away from naïve or vaccinated animals. The kits include reagents for analyzing a sample from a porcine animal for the presence of antibodies to a particular component of a specified TTV virus. Diagnostic kits of the present invention can include as a component a peptide or peptides from ORF1, 2, or 3 which is present in a field strain but not in a vaccine of interest, or vice versa, and selection of such suitable peptide domains is made possible by the extensive amino acid sequencing as provided for in Examples 1 and of the Specification. As is known in the art, kits of the present invention can alternatively include as a component a peptide which is provided via a fusion protein. The term “fusion peptide” or “fusion protein” for purposes of the present invention means a single polypeptide chain consisting of at least a portion of a TTV virus protein, preferably of ORF1, and a heterologous peptide or protein.

### EXAMPLES

### Example 1

### Cloning of Swine TTV Complete Genome

### A. TTV Genotype 2.

DNA was purified from porcine serum using a DNA blood mini kit (Qiagen) per manufacturer's protocol. DNA was eluted from the columns in 50 μl_ Tris-EDTA buffer. DNA was then amplified via random primed rolling circle amplification. Briefly, 5 μL of purified DNA and 100 ng random hexamers (Invitrogen) were then added to 71 μl water and heated at 95 C for 3 min and cooled on ice. One mM dNTP's, 100 ng random hexamers (Invitrogen), 1× phi29 polymerase buffer and 1 μg of phi29 polymerase were then added and the reaction was incubated overnight at 30 C.

One-fifth total volume was digested with EcoRI and electrophoresed on 0.8% E-gel (Invitrogen) to detect presence of 2.7 kB fragment. EcoRI digested material was purified using a Qiagen PCR purification kit following manufacturer's protocol, and ligated into an EcoRI digested/shrimp alkaline phosphatase-treated pGem3zf(+) vector (Promega). Ligated DNA was used to transform chemically competent E. coli DH5a. Transformed E. coli was selected on LB/amp agar plates.

Plasmid DNA was isolated from transformed colonies and digested with EcoRI to confirm presence of an approximately 2.7 kB insert. Four clones (4, 7, 10 and 13) were selected and submitted to ACGT, Inc. for sequencing. Alignment of sequence data indicated that clones 10 and 13 demonstrated homology to TTV published sequence and aligned more closely to TTV genotype 2 than genotype 1. These clones were subsequently named TTV10 and TTV13.

### Analyses of Sequencing Data for PAH TTV Genotype 2.

Nucleotide Alignment of TTV13 (SEQ ID NO:2) and TTV10 (SEQ ID NO:1) to published TTV genotype 2 AY823991 DNA sequence (SEQ ID NO:16).

| | | |
|---|---|---|
| AY823991 | (1) | TCATGACAGGGTTCACCGGAAGGGCTGCAAAA-TTACAGCTAAAACCACA |
| TTV13 | (1) | TAATGACAGGGTTCACCGGAAGGGCTGCAAAA-TTACAGCTAAAACCACA |
| TTV10 | (1) | TAATGACAGGGTTC-CAGGAAGTGCTGCAAAAATTACAGCTAAAACCACA |
| AY823991 | (50) | AGT-CTAACACAATAAACCACAAAGTATTACAGGAAACTGCAATAAATTT |
| TTV13 | (50) | AAT-CTAACACAATAAACCACAAAATATTACAGGAAACTGCAATAAATTT |
| TTV10 | (50) | ACTACTTACACAT--AACCACAAAATATTTCAGGAAACTGCAATAATTTT |
| AY823991 | (99) | AGAAATAAGTTACACATAACCACCA------------AACCACAGGAAAC |
| TTV13 | (99) | AGAAATAAATTACACATAACCACCA------------AACCACAGGAAAC |
| TTV10 | (98) | CAACACACATTGCACAAAACCACAAGATATCAACATAAACCACAGGAAAC |
| AY823991 | (137) | TGTGCAAAAAAGAGGAAATAAATTTCATTGGCTGGGCCTGAAGTCCTCAT |
| TTV13 | (137) | TCTGCAAAAAAGAGGAAATAAATTTCATTGGCTGGTCCATAAGTCCTCAT |
| TTV10 | (148) | TCTGCAAAAAAGAGGAAGTAAATGCTATTGGCTAAATCTGAAGTCTTCAT |
| AY823991 | (187) | TAGAATAATAAAAGAACCAATCAGAAGAACTTCCTCTTTTAGAGTATATA |
| TTV13 | (187) | TAGAATACAAAAAGAACCAATCAGAAACACTTCCTCTTTTAGAGTATATA |
| TTV10 | (198) | TAGCATACACAACCAACCAATCAGAAACACTTCCTCATTTGAAGTATATA |
| AY823991 | (237) | AGTAAGTGCGCAGACGAATGGCTGAGTTTATGCCGCTGGTGGTAGACACG |
| TTV13 | (237) | AGTAAGTGCGCAGACGAATGGCTGAGTTTATGCCGCTGGTGGTAGACACG |
| TTV10 | (248) | AGTAAATGCGCAGACGAATGGCTGAGTTTATGCCGCTGGTGGTAGACACG |
| AY823991 | (287) | AACAGAGCTGAGTGTCTAACCGCCTGGGCGGGTGCCGGAGCTCCTGAGAG |
| TTV13 | (287) | AACAGAGCTGAGTGTCTAACCGCCTGGGCGGGTGCCGGAGCTCCTGAGAG |
| TTV10 | (298) | AACAGAGCTGAGTGTCTAACCGCCTGGGCGGGTGCCGGAGCTCCAGAGAG |
| AY823991 | (337) | CGGAGTCAAGGGGCCTATCGGGCAGGCGGTAATCCAGCGGAACCGGGCCC |
| TTV13 | (337) | CGGAGTCAAGGGGCCTATCGGGCAGGCGGTAATCCAGCGGAACCGGGCCC |
| TTV10 | (348) | CGGAGTCAAGGGGCCTATCGGGCGGGCGGTAATCCAGCGGAACCGGGCCC |
| AY823991 | (387) | CCC-TCGATGGAAGAAAGATGGCTGACGGTAGCGTACTGCGCACACGGAT |
| TTV13 | (387) | CCCCTCCATGGAAGAAAGATGGCTGACGGTAGCGTACTGCGCCCACGGAT |
| TTV10 | (398) | CCC-TCCATGGAGGAGAGATGGCTGACGGTAGCGTACGCCGCCCACGGAT |
| AY823991 | (436) | TATTCTGCAGCTGTAAAGACCCGAAAAAACATCTTGAAAAATGCCTTACA |
| TTV13 | (437) | TATTCTGCGACTGTAAAGACCCGAAAAAACATCTTGAAAAATGCCTTACA |
| TTV10 | (447) | TATTCTGCGCCTGCAGTAAGCCCAAAGACCACCTTGAAAAATGCCTTTCC |
| AY823991 | (486) | GACGCTATCGCAGACGCCGAAGAAGACCGACACGGAGATGGAGGCACCGG |
| TTV13 | (487) | GACGCTATCGCAGACGCCGAAGGAGACCGACAAGAAGATGGAGGCACCGG |
| TTV10 | (497) | ACCGCTATCGCCGACGCCGAAGGAGACCCACCAGGAGATGGAGGAGAAGG |
| AY823991 | (536) | AGGTGGAGACGCTACTTTCGATATCGGTATCGACGCGCTCCTCGCCGCCG |
| TTV13 | (537) | AGGTGGAGACGCTACTTTCGATATCGGTATCGACGCGCTCCTCGCCGCCG |
| TTV10 | (547) | AGGTTCCAGCGCTACTTTCGATATCGGTATAGACGCGCTCCTCGCCGCCG |
| AY823991 | (586) | CCG---CACAAAGGTAAGGAGACGGAGG---AAAAAAGCTCCGGTCATAC |
| TTV13 | (587) | CCG---CACAAAGGTAAGGAGACGGAGG---AGGAAAGCTCCGGTCATAC |
| TTV10 | (597) | CCGACGCTACAAGGTAAGGAGACGGAGGGTTAAAAAGGCTCCGGTCATTC |
| AY823991 | (630) | AATGGTTCCCTCCTAGCCGGAGAACCTGCCTCATAGAGGGATTTTGGCCG |
| TTV13 | (631) | AATGGAACCCTCCTAGCCGGAGGACCTGCCTCATAGAGGGGTTCTGGCCG |
| TTV10 | (647) | AATGGTTCCCCCCAACAGTCAGAAACTGTTTTATCAAGGGAATCTGGCCG |
| AY823991 | (680) | TTGAGCTACGGACACTGGTTCCGTACCTGTCTCCCCTTTAGGCGGTTAAA |
| TTV13 | (681) | TTGAGCTACGGACACTGGTTCCGTACCTGTCTCCCCTTTAGAAGAAAAAA |
| TTV10 | (697) | TTGAGCTACGGACACTGGCTCCGTACCTGTCTCCCTATGAGAAAAGAAAA |
| AY823991 | (730) | TGGACTAGTATTCCCGGGTGGAGGTTGTGACTGGAGCCAGTGGAGTTTAC |
| TTV13 | (731) | TGGACTAATATTTACGGGAGGAGGTTGTGACTGGACTCAGTGGAGCTTAC |
| TTV10 | (747) | CGGACTCATATTCCTAGGAGGTGGCATAGACTGGACTGTCTGGAGTTTAC |
| AY823991 | (780) | AAAACCTTTACAATGAAAAACTTAACTGGAGAAATATATGGACAGCTAGT |
| TTV13 | (781) | AAAACCTTTATCATGAAAAACTAAACTGGAGAAATATATGGACAGCTAGT |
| TTV10 | (797) | AGAATCTATACCATGAAAAACTAAACTGGAGGAATGTGTGGACTTCTTCA |
| AY823991 | (830) | AATGTTGGAATGGAATTCGCTAGATTTTTAAAAGGAAAGTTTTACTTTTT |
| TTV13 | (831) | AACGTGGGAATGGAATTCGCTAGATTTTTAAAAGGAAAATTCTACTTTTT |
| TTV10 | (847) | AATGATGGCATGGAGTTCGCTAGATTCAGATATGCAAAGTTTAAATTTTT |
| AY823991 | (880) | CAGACATCCATGGAGAAATTATATAATAACTTGGGATCAAGATANACCAT |
| TTV13 | (881) | TAGACATCCTTGGAGAAACTATATAGTGACTTGGGATCAGGACATTCCTT |
| TTV10 | (897) | TAGACACACAACCAGATCCTACGTAGTAACATGGGACCAAGACATACCAT |
| AY823991 | (930) | GCAGGCCACTACCTTATCAAAACCTGCATCCACTCCTAATGCTACTAAAA |
| TTV13 | (931) | GTAAACCTTTACCATATCAGAACTTACACCCATTATTAATGCTATTAAAA |
| TTV10 | (947) | GTAAACCTTTACCATACACAAATTTACATCCATTTGTAATGCTTCTAAAA |
| AY823991 | (980) | AAACAGCACAAAATTGTACTTTCACAGCAAAACTGTAACCCAAACAGAAA |
| TTV13 | (981) | AAACAACACAAATTAGTACTCTCACAACAAAACTGTAACCCTAACAGAAA |
| TTV10 | (997) | AAACATCATAAAGTAGTTCTAAGCAAACAAGACTGTAATCCTAGAAAAAT |
| AY823991 | (1030) | ACAAAAACCTGTCACATTAAAATTCAAACCTCCGCCAAAACTAACATCAC |
| TTV13 | (1031) | ACAAAAACCTGTAACTTTAAAATTCAGACCGCCACCAAAACTAACTTCAC |
| TTV10 | (1047) | GGACAAACCAGTCACCTTAAAAATAAAGCCACCACCAAAACTCACATCAC |
| AY823991 | (1080) | AATGGAGACTAAGTAGAGAATTAGCAAAGATGCCACTAATAAGACTTGGA |
| TTV13 | (1081) | AATGGAGACTAAGTAGAGAATTAGCAAAAATGCCACTCATTAGACTAGGA |
| TTV10 | (1097) | AGTGGAGACTAAGCAGAGAATTATCAAAAATACCGCTCTTAAGACTAGGA |
| AY823991 | (1130) | GTAAGCTTTATAGACCTAACAGAACCATGGGTAGAAGGGTGGGGAAATGC |
| TTV13 | (1131) | GTTAGTTTTATAGACTTAACAGAACCGTGGCTAGAAGGTTGGGGAAATGC |
| TTV10 | (1147) | GTTTCTTTAATAGACTTCAGAGAACCATGGGTTGAAGGTTTTGGAAATGC |
| AY823991 | (1180) | ATTTTATTCCGTGCTAGGATATGAAGCAGTAAAAGACCAAGGACACTGGT |
| TTV13 | (1181) | ATTTTACTCAGTACTAGGATATGAAGCCATAAAAGAACAAGGACACTGGT |
| TTV10 | (1197) | CAAACTGGACACAAATAAAATACTATTGGATCTATGACACGGGAGTAGGA |
| AY823991 | (1230) | CAAACTGGACACAAATAAAATACTATTGGATCTATGACACGGGAGTAGGA |
| TTV13 | (1231) | CAAATTGGTCACAAATTAAATATTACTGGATATATGATACAGGAGTAGGA |
| TTV10 | (1247) | GCGCTTGGTGCCAATGTAAATACTTCTGGATATATGATACCGGAGTAAAT |
| AY823991 | (1280) | AATGCAGTATATGTTATACTATTAAAAAAAGACGTTACTGATAATCCAGG |
| TTV13 | (1281) | AATGCTGTATATGTAGTTATGCTAAAACAAGATGTAGACGACAACCCAGG |
| TTV10 | (1297) | AATCATGTATATGTAGTCATGTTAAACAAAGACGCAGGAGATAATGCAGG |
| AY823991 | (1330) | AAACATGGCAACAACCTTTAAAGCATCAGGAGGACAGCATCCAGATGCAA |
| TTV13 | (1331) | AAAAATGGCATCAACATTTAAAACAACTCAGGGACAACATCCCAATGCTA |
| TTV10 | (1347) | AGACCTAATAACAA------------------------ATCAAAACTCAA |
| AY823991 | (1380) | TAGATCACATTGAATTGATAAACCAAGGATGGCCTTACTGGTTATACTTT |
| TTV13 | (1381) | TAGATCACATAGAATTAATAAATGAAGGATGGCCGTACTGGTTATACTTT |
| TTV10 | (1373) | TAGCACACATAGAACAGATAGGAGAAGGTTATCCATACTGGTTATATTTT |
| AY823991 | (1430) | TATGGTAAAAGTGAACAAGACATTAAAAAAGAGGCACAC---AGCGCAGA |
| TTV13 | (1431) | TTTGGTAAAAGTGAACAAGACATAAAAAAGGAAGCACAT---AGCGCTGA |
| TTV10 | (1423) | TTTGGAAGATCTGAAAGAGACTTAAAAGCACTAGCAACTTCAAACACAAA |
| AY823991 | (1477) | AATATCAAGAGAATATACTAGAGACCCAAAATCTAAAAAACTAAAAATAG |
| TTV13 | (1478) | AATAGCAAGAGAATATGCTACAAATCCAAAATCAAAAAAACTAAAAATAG |
| TTV10 | (1473) | CATAAGAAACGAATTCAATACTAATCCTAACAGCAAAAAATTAAAAATAG |
| AY823991 | (1527) | GAATAGTAGGATGGGCATCTTCAAACTACACAACAACAGGCAGTGATCAA |
| TTV13 | (1528) | GAATAGTAGGATGGGCATCCTCTAACTTCACAACACCAGGCAGTTCACAA |
| TTV10 | (1523) | CTGTAATAGGATGGGCTAGCAGTAACAACACAGCACAAGATAGTACACAA |
| AY823991 | (1577) | AACAGTGGTGGATCAACATCAGCTATACAAGGTGGATATGTAG-----CA |
| TTV13 | (1578) | AACTCAGGGGGAAATATAGCAGCAATACAAGGAGGATACGTAG-----CA |
| TTV10 | (1573) | ---------GGAGCGAATACTCCAATAGAAGGAACATATTTAATATCACA |
| AY823991 | (1622) | TATGC-AGG-GTCCGGGGTCA--------TAGGAGCAGGGTCAATAGGAA |
| TTV13 | (1623) | TGGGC-AGGAGGACAAGGAAAACTAAATCTAGGAGCAGGATCAATAGGAA |
| TTV10 | (1614) | TGTGCTACAAACATCAGGACATACAG---CAGGAGCAGCACAAATAAATA |
| AY823991 | (1662) | ATTTATATCAACAAGGATGGCCATCTAATCAAAACTGGCCTAATACAAAC |
| TTV13 | (1672) | ATTTGTACCAACAAGGATGGCCATCAAATCAAAACTGGCCAAATACAAAC |
| TTV10 | (1661) | ACCTATTCGCCTCTGGATGGCCTAACTCTCAAAACTATCCACCTTTAAAT |
| AY823991 | (1712) | AGAGACAAAACAAACTTTGACTGGGGAATACGAGGACTATGTATACTCAG |
| TTV13 | (1722) | AGAGACGAAACTAACTTTGATTGGGGACTCAGATCACTTTGTATACTAAG |
| TTV10 | (1711) | CTAGACAAAAACAACTTTGACTGGGGAAAAAGAGCGCTATGTATACTAAG |
| AY823991 | (1762) | AGATAACATGCACTTAGGAAGCCAAGAATTAGATGATGAATGCACAATGC |
| TTV13 | (1772) | AGATAACATGCAATTAGGAAATCAAGAATTAGATGATGAATGTACCATGC |
| TTV10 | (1761) | AAACAACATGAAAATTGGAAACCAAAATTTAGATGATGAGACCACTATGT |
| AY823991 | (1812) | TCACATTGTTCGGACCCTTTGTAGAAAAAGCAAATCCAATATTTGCAACA |
| TTV13 | (1822) | TCTCACTCTTTGGACCTTTTGTAGAAAAAGCAAATCCAATATTTGCAACA |
| TTV10 | (1811) | TTGCCCTCTTCGGACCCTTGGTAGAAAAAGCAAA-CTGGGAAGGCCTAGA |
| AY823991 | (1862) | ACAGACCCTAAATTCTTTAAACCTGAACTCAAAGACTATAATATAATCAT |
| TTV13 | (1872) | ACAGACCCTAAATACTTTAAACCAGAACTAAAAGACTATAATTTAATCAT |
| TTV10 | (1860) | AAAAATACCAGAA--CTAAAACCAGAACTCAAAGACTATAATATCTTAAT |
| AY823991 | (1912) | GAAATATGCCTTTAAATTTCAGTGGGGAGGACATGGCACAGAAAGATTTA |
| TTV13 | (1922) | GAAATATGCCTTTAAATTCCAGTGGGGAGGACATGGCACAGAAAGATTTA |
| TTV10 | (1908) | GAGATATAACTTTCGCTTTCAGTGGGGCGGACACGGAACAGAGACCTTCA |
| AY823991 | (1962) | AAACCAACATCGGAGACCCCAGCACCATACCCTGCCCCTTCGAACCCGGG |
| TTV13 | (1972) | AAACAACCATCGGAGACCCCAGCACCATACCCTGCCCCTTCGAACCCGGG |
| TTV10 | (1958) | AAACAAGTATTGGAGACCCCAGCCAAATACCCTGTCCCTACGGACCAGGT |
| AY823991 | (2012) | GACCGCTTCCA-CAGCGGGATACAAGACCCCTCCAAGGTACAAAACACCG |
| TTV13 | (2022) | GACCGCTTCCA-CAGCGGGATACAAGACCCCTCCAAGGTACAAAACACCG |
| TTV10 | (2008) | GAAGCCCCCCAACACCTTGTCAGGA-ACCCCTCCAAGGTACACGAGGGGG |
| AY823991 | (2061) | TCCTCAACCCCTGGGACTATGACTGTGATGGGATTGTTAGAAAAGATACT |
| TTV13 | (2071) | TCCTCAACCCCTGGGACTATGACTGTGATGGGATTGTTAGAAAAGATACT |
| TTV10 | (2057) | TCCTCAATGCGTGGGATTATGACTATGATGGAATTGTTAGAAAAGACACT |
| AY823991 | (2111) | CTCAAAAGACTTCTCGAACTCCCCACAGAGACAGAGGAGGAGGAGAAGGC |
| TTV13 | (2121) | CTCAAAAGACTTCTCGAACTCCCCACAGAGACAGAGGAGGAGGAGAAGGC |
| TTV10 | (2107) | CTCAAAAGACTGCTTGCCATCCCCACAGACTC---GGAGGAGGAGAAAGC |
| AY823991 | (2161) | GTACCCACTCCTTGGACAAAAAACAGAGAAAGAGCCATTATCAGACTCCG |
| TTV13 | (2171) | GTACCCACTCCTTGGACAAAAAACAGAGAAAGAGCCATTATCAGACTCCG |
| TTV10 | (2154) | GTACCCGCTCGCTGGACCCAAAACAGAGAAATTGCCCTCCTCAGACGAAG |
| AY823991 | (2211) | ACGAAGAGAGCGTTATCTCAAGCACGAGCAGTGGATCCTCTCAAGAA--- |
| TTV13 | (2221) | ACGAAGAGAGCGTTATCTCAAGCACGAGCAGTGGATCCGATCAAGAA--- |
| TTV10 | (2204) | AAGGAGAGAGCGATATCAGTTCTTCGAGCGACTCATCGACGCAAGAAAGC |
| AY823991 | (2258) | GAAGAAACGCAGAGAC---GAAGACACCACAAGCCAAGCAAGCGACGACT |
| TTV13 | (2268) | GAAGAGACGCAGAGAC---GAAAGCACCACAAGCCAAGCAAGCGACGACT |
| TTV10 | (2254) | GAAGAAGAGAAGAGATACAGAAGACGACACAAGCCCTCAAAGCGAAGACT |
| AY823991 | (2305) | CCTCAAGCACCTCCAGCGGGTGGTAAAGAGGATGAAAACACTGTGATAGA |
| TTV13 | (2315) | CCTCAAGCACCTCCAGCGGGTGGTAAAGAGGATGAAAACACTGTGATAGA |
| TTV10 | (2304) | CCTCCAGCATGTCCAGCGACTGGTGAAGAGATTCAGGACCCT---ATAGA |
| AY823991 | (2355) | TAAATATAGAAACCTAGCAGACCCCTCACTCAATGTCACAGGACACATGG |
| TTV13 | (2365) | TAAATACAGAAACCTAGCAGACCCCTCACTCAATGTCACAGGACACATGG |
| TTTV10 | (2351) | CAAATACAGAAACTTAGCAGACCCCTCATTAAATGTCACAGGACATTTTG |
| AY823991 | (2405) | AAAAATTCATGCAGTTACATATTCAAAACGTACAAGAAATAAGAGCTAAA |
| TTV13 | (2415) | AAAAATTCATGCAACTACATATCCAAAACATACAAGAAATAAGAGCTAAA |
| TTV10 | (2401) | AACACTTCTGCCGCTTACACTATAAAAACATAGCAGAAATCAGAGCTAGA |
| AY823991 | (2455) | AATGCTAAAAAATCCCTCAATAAACTTTACTTTTCTGATTAATAGCGGCC |
| TTV13 | (2465) | AATGCTAAAAAATCCCTCAATAAACTTTACTTTTCTGATTAATAGCGGCC |
| TTV10 | (2451) | AATGCCAAAAAAAACCTCAATAAACTATACTTTTCAGACTAAAAGAAG-- |
| AY823991 | (2505) | TCCTGTGTCCAACCTATTTTTCCTAAACCCCTTCAAAATGGCGGGCGGGA |
| TTV13 | (2515) | TCCTGTGTCCAATCTATTTTTTTAAACACCCTTCAAAATGGCGGGAGGGA |
| TTV10 | (2499) | TTT--------ATTTCTTTATTTAAAACACC------------------- |
| AY823991 | (2555) | CACAAAATGGCGGAGGGACTAAGGGGGGGGCAAGCCCCCCTNNNNNNNNN |
| TTV13 | (2565) | CACAAAATGGCGGAGGGACTAAGGG-----------------TGNNNNNN |
| TTV10 | (2522) | -----------------ACTA----------------------GAGGGCG |
| AY823991 | (2605) | NNNNNNNNNNNNNNNNNNGGGGGGCGACCCCCCCGCACCCCCCCCTGCGG |
| TTV13 | (2598) | NNNNNNNNNTAGGCTCTTCG---------CCCCCGCACCCCCCC-TGCGG |
| TTV10 | (2533) | TAGCGGGGGGGGGACC-------------CCCCTGCACCCCCCCATGCGG |
| AY823991 | (2655) | GGGCTCCGCCCCCTGCACCCCCGGGAGGGGGGGAAACCCCCCCTCAACCC |
| TTV13 | (2638) | GGGCTCCGCCCCCTGCACCCCCGGGAGGGGGGGAAACCCCCCCTCAACCC |
| TTV10 | (2570) | GGGCTCCGCCCCCTGCACCCCCGGGAGGGGGGGAAACCCCCCCTCAACCC |
| AY823991 | (2705) | CCCGCGGGGGG-CAAGCCCCCCTGCACCCCCC- |
| TTV13 | (2688) | CCCGCGGGGGG-CAAGCCCCCCTGCACCCCCC- |
| TTV10 | (2620) | CCCGCGGGGGGGCAAGCCCCCCTGCACCCCCCC |

| | AB076001 | AY823990 | AY823991 | TTV13 | TTV10 |
|---|---|---|---|---|---|
| AB076001 | | 72 | 49 | 49 | 48 |
| AY823990 | | | 48 | 48 | 48 |
| AY823991 | | | | 92 | 76 |
| TTV13 | | | | | 76 |
| TTV10 | | | | | |

### Nucleotide Identity

| | AY823991 | TTV13 | TTV10 |
|---|---|---|---|
| AY823991 | | 92 | 76 |
| TTV13 | | | 76 |

TTV 13 shows 92% identity when compared with previously published AY823991 sequence. However, TTV10 only show 76% similarity between either AY823991 or TTV13 and may be considered a separate genotype.

Amino Acid Alignment of PAH TTV Genotype ORF1 for TTV10 (SEQ ID NO:14) and TTV13 (SEQ ID NO:15) with AY823991 ORF1 (SEQ ID NO:8).

| | | |
|---|---|---|
| AY823991 Orf1 | (1) | MPYRRYRRRRRRPTRRWRHRRWRRYFRYRYRRAPRRRR-TKVRRR-RKKA |
| TTV10Orf1 | (1) | MPFHRYRRRRRRPTRRWRRRRFQRYFRYRYRRAPRRRRRYKVRRRRVKKA |
| TTV13ORF1 | (1) | MPYRRYRRRRRRPTRRWRHRRWRRYFRYRYRRAPRRRR-TKVRRR-RRKA |
| AY823991 Orf1 | (49) | PVIQWFPPSRRTCLIEGFWPLSYGHWFRTCLPFRRLNGLVFPGGGCDWSQ |
| TTV10Orf1 | (51) | PVIQWFPPTVRNCFIKGIWPLSYGHWLRTCLPMRKENGLIFLGGGIDWTV |
| TTV13ORF1 | (49) | PVIQWNPPSRRTCLIEGFWPLSYGHWFRTCLPFRRKNGLIFTGGGCDWTQ |
| AY823991 Orf1 | (99) | WSLQNLYNEKLNWRNIWTASNVGMEFARFLKGKFYFFRHPWRNYIITWDQ |
| TTV10Orf1 | (101) | WSLQNLYHEKLNWRNVWTSSNDGMEFARFRYAKFKFFRHTTRSYVVTWDQ |
| TTV13ORF1 | (99) | WSLQNLYHEKLNWRNIWTASNVGMEFARFLKGKFYFFRHPWRNYIVTWDQ |
| AY823991 Orf1 | (149) | DIPCRPLPYQNLHPLLMLLKKQHKIVLSQQNCNPNRKQKPVTLKFKPPPK |
| TTV10Orf1 | (151) | DIPCKPLPYTNLHPFVMLLKKHHKVVLSKQDCNPRKMDKPVTLKIKPPPK |
| TTV13ORF1 | (149) | DIPCKPLPYQNLHPLLMLLKKQHKLVLSQQNCNPNRKQKPVTLKFRPPPK |
| AY823991 Orf1 | (199) | LTSQWRLSRELAKMPLIRLGVSFIDLTEPWVEGWGNAFYSVLGYEAVKDQ |
| TTV10Orf1 | (201) | LTSQWRLSRELSKIPLLRLGVSLIDFREPWVEGFGNAFFSTLGYEADKSN |
| TTV13ORF1 | (199) | LTSQWRLSRELAKMPLIRLGVSFIDLTEPWLEGWGNAFYSVLGYEAIKEQ |
| AY823991 Orf1 | (249) | GHWSNWTQIKYYWIYDTGVGNAVYVILLKKDVTDNPGNMATTFKASGGQH |
| TTV10Orf1 | (251) | LKTSAWCQCKYFWIYDTGVNNHVYVVMLNKDAGDNAGDLITNQNS----- |
| TTV13ORF1 | (249) | GHWSNWSQIKYYWIYDTGVGNAVYVVMLKQDVDDNPGKMASTFKTTQGQH |
| AY823991 Orf1 | (299) | PDAIDHIELINQGWPYWLYFYGKSEQDIKKEAHS-AEISREYTRDPKSKK |
| TTV10Orf1 | (296) | ---IAHIEQIGEGYPYWLYFFGRSERDLKALATSNTNIRNEFNTNPNSKK |
| TTV13ORF1 | (299) | PNAIDHIELINEGWPYWLYFFGKSEQDIKKEAHS-AEIAREYATNPKSKK |
| AY823991 Orf1 | (348) | LKIGIVGWASSNYTTTGSDQNSGG-STSAIQGGYVAYAGSG---VIGAGS |
| TTV10Orf1 | (343) | LKIAVIGWASSNNTAQDSTQGANTPIEGTYLISHVLQTSGH---TAGAAQ |
| TTV13ORF1 | (348) | LKIGIVGWASSNFTTPGSSQNSGG-NIAAIQGGYVAWAGGQGKLNLGAGS |
| AY823991 Orf1 | (394) | IGNLYQQGWPSNQNWPNTNRDKTNFDWGIRGLCILRDNMHLGSQELDDEC |
| TTV10Orf1 | (390) | INNLFASGWPNSQNYPPLNLDKNNFDWGKRALCILRNNMKIGNQNLDDET |
| TTV13ORF1 | (397) | IGNLYQQGWPSNQNWPNTNRDETNFDWGLRSLCILRDNMQLGNQELDDEC |
| AY823991 Orf1 | (444) | TMLTLFGPFVEKANPIFATTDPKFFKPELKDYNIIMKYAFKFQWGGHGTE |
| TTV10Orf1 | (440) | TMFALFGPLVEKAN-WEGLEKIPELKPELKDYNILMRYNFRFQWGGHGTE |
| TTV13ORF1 | (447) | TMLSLFGPFVEKANPIFATTDPKYFKPELKDYNLIMKYAFKFQWGGHGTE |
| AY823991 Orf1 | (494) | RFKTNIGDPSTIPCPFEPGDRFHSGIQDPSKVQNTVLNPWDYDCDGIVRK |
| TTV10Orf1 | (489) | TFKTSIGDPSQIPCPYGPGEAPQHLVRNPSKVHEGVLNAWDYDYDGIVRK |
| TTV13ORF1 | (497) | RFKTTIGDPSTIPCPFEPGDRFHSGIQDPSKVQNTVLNPWDYDCDGIVRK |
| AY823991 Orf1 | (544) | DTLKRLLELPTETEEEEKAYPLLGQKTEKEPLSDSDEESVISSTSSGSSQ |
| TTV10Orf1 | (539) | DTLKRLLAIPTDSEEE-KAYPLAGPKTEKLPSSDEEGESDISSSSDSSTQ |
| TTV13ORF1 | (547) | DTLKRLLELPTETEEEEKAYPLLGQKTEKEPLSDSDEESVISSTSSGSDQ |
| AY823991 Orf1 | (594) | EEETQR--RRHHKPSKRRLLKHLQRVVKRMKTL-- |
| TTV10Orf1 | (588) | ESEEEKRYRRRHKPSKRRLLQHVQRLVKRFRTL-- |
| TTV13ORF1 | (597) | EEETQR--RKHHKPSKRRLLKHLQRVVKRMKTL-- |

Amino Acid Alignment of TTV10 TTV13 ORF with Published Sequence

| | AY823991 Orf1 | TTV10Orf1 | TTV13ORF1 |
|---|---|---|---|
| AY823991 Orf1 | | 65 | 92 |
| TTV10Orf1 | | | 66 |
| TTV13ORF1 | | | |

On the amino acid level, TTV10 ORF demonstrates only 65% homology to the published sequence and may represent a unique phenotype of TTV

### Cloning of TTV Genotype 2 ORF1 for Baculovirus Expression

Based on sequence data derived above, primers were designed to clone the ORF from TTV10 and TTV13 for expression in baculovirus using the Invitrogen Gateway® system. Primer sequences were:

### For TTV13 ORF: Ttv13Rev1211: 5′ cgt act cga gtc aca gtg ttt tca tcc (SEQ ID NO:26); TTV13For1211: 5′ cta ggt acc atg cct tac aga cgc tat (SEQ ID NO:27)

For TTV10 ORF: tt10for1207: 5′ cta ggt acc atg cct ttc cac cgc tat (SEQ ID NO:28) and ttvrev1207: cgt act cga get ata ggg tcc tga at (SEQ ID NO:29)

Since the EcoRI cloning into pGem resulted in interrupting the reading frame of the ORF1, the TTV insert in pGem was isolated by EcoRI digestion, gel-purified and re-circularized using standard ligation conditions. Following an overnight ligation at 4° C., ligase was inactivated at 65° C., and the reaction was purified using QuiQuick purification kit (Qiagen) following the manufacturer's protocol.

TTVORF13 was the PCR amplified using re-circularized TTV13 genomic DNA with Expand Hi-Fidelity® enzyme (Roche) using the above described TTV13 forward and reverse primers (0.15 μM each), 0.2 mM dNTP's in 1× Hi Fidelity enzyme buffer. PCR conditions were: 1 cycle at 4 min, 95° C.; 35 cycles with 94° C., 15 sec denaturation, 55° C., 30 sec anneal, and 68° C. 1.5 min extension; and 1 cycle of 72° C., 7 min extension.

Similarly, TTVORF10 was PCR amplified using re-circularized TTV10 genomic DNA with Expand Hi-Fidelity® enzyme (Roche) using the above described TTV10 forward and reverse primers (0.15 μM each) 0.2 mM dNTP's in 1× Hi Fidelity enzyme buffer. PCR conditions were: 1 cycle at 4 min, 95° C.; 35 cycles with 94° C., 15 sec denaturation, 56° C., 30 sec anneal, and 68° C. 1.5 min extension; and 1 cycle of 72° C., 7 min extension.

PCR products were purified using QiaQuick PCR purification kit (Qiagen) following the manufacturer's protocol. Both PCR TTV100rf1 and TTV13Orf1 products and the Gateway entry plasmid, pENTR3C, were digested with KpnI. Digested DNA was purified using QIAquick PCR amplification kit and subsequently digested with Xhol. Following QIAquick purifications, the TTV10 ORF1 or the TTV13ORF1 DNAs were ligated into pENTR3C using standard ligation procedures. Following a 2 hour ligation at room temperature, ligated DNA was used to transform chemically competent E. coli DH5α. Transformed colonies were selected using Kanamycin. Plasmid was purified from transformed E. coli and ORF1 DNA insertion was verified by restriction fragment analysis.

pENTR3C plasmids containing TTV10 ORF1 or TTV13 ORF1 were then inserted into Invitrogen destination vectors pDEST10 or pDEST 20 encoding a His6X or a GST protein N-terminal to the TTV Orf1 reading frame. Recombinant pDEST vectors containing the open reading frame of TTV Orf1 were used to transform DH10Bac E. coli. Recombinant bacmid DNA was isolated and used for transfection of SF9 cells following standard protocol. Recombinant baculovirus containing the native Orf1 were isolated by plaque purification. Confirmation of recombinant baculovirus was performed using PCR.

### Native TTVOrf1 Construction for Baculovirus Expression.

Standard PCR was used to incorporate a BamH1 restriction site upstream from the initiation codon in TTV10 Orf1 or an XbaI restriction site upstream from the initiation codon in TTV Orf13. These constructs were cloned into pFastBac transfer vector and used to transform E. coli DH10Bac. The resultant recombinant bacmids were subsequently used to transfect SF9 cells. Recombinant baculovirus containing the native Orf1 were isolated by plaque purification. Confirmation of recombinant baculovirus was performed using PCR.

Cloning of TTV Genotype 2 ORF1 for E. coli Expression.

Full-length TTVOrf10 was also cloned into a PGex-6p-1 vector for expression of a GST-fusion protein in a bacterial system. The TTV ORFs contain an arginine rich amino terminus. To determine if protein production could be increased in a bacterial expression system, the arginine rich segment was removed from TTVOrf13 at a convenient restriction site (EcoR1) located at nucleotide 368 of the Orf1 open reading frame and was in frame with the GST coding region of pGex-6p-1. This clone resulted in the removal of 100 amino-terminal amino acids containing a highly enriched arginine segment.

### B. TTV Genotype 1.

Total cellular DNA from porcine bone marrow was amplified by rolling circle amplification following procedures described above, except that single-stranded binding protein was added to improve the efficiency of the amplification reaction. Amplification products were digested with EcoR1, purified using a QIAquick PCR purification kit (Qiagen), and ligated into pGem3zf(+) vector which had been previously treated with shrimp alkaline phosphatase. Recombinant vector containing putative TTV genomic DNA was selected based on restriction digests with EcoR1 and/or BamH1. Plasmids containing approximately 2.7 kB inserts were purified and submitted to ACGT, Inc. for sequencing of the ORF1 sequences to confirm genotype. The complete genome, i.e. the region containing the high G/C rich region, was not sequenced to entirety.

### Analyses of Sequencing Data for PAH TTV Genotype 1

Nucleotide alignment of PAH TTV7 (SEQ ID NO:4), TTV17 (SEQ ID NO:5), TTV21 (SEQ ID NO:6), and TTV27 (SEQ ID NO:3) with published sequence, AY823990 (SEQ ID NO:17).

| | | |
|---|---|---|
| AY823990 | (1) | TACACTTTGGGGTTCAGGAGGCTCAATTTGGCTCGCTTCGCTCGCACCAC |
| ttvg1-7 | (1) | TACACTTCCGGGTTCAGGAGGCTCAATTTGGCTCGCTTCGCTCGCACCAC |
| ttvGT1-17 | (1) | TACACTTCCGGGTTCAGGAGGCTCAATTTGGCTCGCTTCGCTCGCACCAC |
| ttvGT1-21 | (1) | TACACTTCCGGGTTCAGGAGGCTCAATTTGGCTCGCTTCGCTCGCACCAC |
| ttvgt1-27 | (1) | TACACTTCCGGGTTCAGAGGGCTCAATTTGGCTCGCTTCGCTCGCACCAC |
| AY823990 | (51) | GTTTGCTGCCAGGCGGACCTGATTGAAGACTGAAAACCGTTAAATTCAAA |
| ttvg1-7 | (51) | GTTTGCTGCCAGGCGGACCTGTTTGAAGACTGAAAACCGTTAAATTCAAA |
| ttvGT1-17 | (51) | GTTTGCTGCCAAGCGGACCTGATTGAAGACTGAAAACCGTTACATTCAAA |
| ttvGT1-21 | (51) | GTTTGCTGCCAGGCGGACCTGATTGAAGACTGAAAACCGTTAAATTCAAA |
| ttvgt1-27 | (51) | GTTTGCTGCCAGGCGGACCTGATTGAAGACTGAAAACCGTTAAGTTCAAA |
| AY823990 | (101) | ATTGAAAAGGGCGGGCAAA-ATGGCGGACAGGGGGCGGAGTTTATGCAAA |
| ttvg1-7 | (101) | TTTGAAATTGGCGGT-AAACATGGCGGAAGGGGGGCGGAGTATATGCAAA |
| ttvGT1-17 | (101) | TTTGAAAATGGCGCCCAAACATGGCGGATGTGGG-CGGAGTATATGCAAA |
| ttvGT1-21 | (101) | TTTGAAATTGGCGGT-AAATATGGCGGAAGGGGGGCGGAGTATATGCAAA |
| ttvgt1-27 | (101) | TTTGAAAATGGCGCCCAAACATGGCGGAG-GGGGGCGGAGTTTATGCAAA |
| AY823990 | (150) | TTAATTTATGCAAAGTAGGAGGAGCTCGATTTTAATTTATGCAAAGTAGG |
| ttvg1-7 ... | (150) | TTAATTTATGCAAAGTAGGAGGAGCTCGATTTTAATTTATGCAAAGTAGG |
| ttvGT1-17... | (150) | TTAATTTATGCAAAGTAGGAGGAGCTCGATTTTAATTTATGCAAAGTAGG |
| ttvGT1-21... | (150) | TTAATTTATGCAAAGTAGGAGGAGCTCGATTTTAATTTATGCAAAGTAGG |
| ttvgt1-27... | (150) | TTAATTTATGCAAAGTAGGAGGAGCTCCATTTTAATTTATGCAAAGTAGG |
| AY823990 | (200) | AGGAGTCAAATCTGATTGGTCGGGAGCTCAAGTCCTCATTTGCATAGGGT |
| ttvg1-7 ... | (200) | AGGAGTCAAATCTGATTGGTCGGGAGCTCAAGTCCTCATTTGCATAGGGT |
| ttvGT1-17... | (200) | AGGAGTCACTTCTGATTGGTCGGGAACTCAAGCCCTCATTTGCATAGGGT |
| ttvGT1-21... | (200) | AGGAGTCAAATCTGATTGGTCGGGAGCTCAAGTCCTCATTTGCATAGGGT |
| ttvgt1-27... | (200) | AGGAGTCACTTCTGATTGGTCGGGAGCTCAAGTCCTCATTTGCATAGGGT |
| AY823990 | (250) | GTAACCAATCAGAATTAAGGCGTTCCCACGAAAGCGAATATAAGTAGGTG |
| ttvg1-7 ... | (250) | GTAACCAATCAGAATTAAGGCGTGCCCACTAAAGTGAATATAAGTAAGTG |
| ttvGT1-17... | (250) | GTAACCAATCAGAATTAAGGCGTTCCCCGTGAAGTGAATATAAGTAAGTA |
| ttvGT1-21... | (250) | GTAACCAATCAGAATTAAGGCGTGCCCACTAAAGTGAATATAAGTGAGTG |
| ttvgt1-27... | (250) | GTAACCAATCAAACTTAAGGCGTTCCCACTAAAGTGAATATAAGTAAGTG |
| AY823990 | (300) | AGGTTCCGAATGGCTGAGTTTATGCCGCCAGCGGTAGACAGAACTGTCTA |
| ttvg1-7 ... | (300) | CAGTTCCGAATGGCTGAGTTTATGCCGCCAGCGGTAGACAGAACTGTCTA |
| ttvGT1-17... | (300) | AAGTTCCGAATGGCTGAGTTTATGCCGCCAGCGGTAGACAGAACTGTCTA |
| ttvGT1-21... | (300) | CAGTTCCGAATGGCTGAGTTTATGCCGCCAGCGGTAGACAGAACTGTCTA |
| ttvgt1-27... | (300) | CGGTTCCGAATGGCTGAGTTTATGCCGCCAGCGGTAGACAGAACTGTCTA |
| AY823990 | (350) | GCGACTGGGCGGGTGCCGGAGGATCCCTGATCCGGAGTCAAGGGGCCTAT |
| ttvg1-7 ... | (350) | GCGACTGGGCGGGTGCCGGAGGATCCCAGATCCGGAGTCAAGGGGCCTAT |
| ttvGT1-17... | (350) | GCGACTGGGCGGGTGCCGAAGGATCCCAGATCCGGAGTCAAGGGGCCTAT |
| ttvGT1-21... | (350) | GCGACTGGGCGGGTGCCGGAGGATCCCAGATCCGGAGTCAAGGGGCCTAT |
| ttvgt1-27... | (350) | GCGACTGGGCGGGTGCCGGAGGATCCCTGATCCGGAGTCAAGGGGCCTAT |
| AY823990 | (400) | CGGGCAGGAGCAGCTAGGCGGAGGGCCTATGCCGGAACACTGGGAGGAAG |
| ttvg1-7 ... | (400) | CGGGCAGGAGCAGCTGAGCGGAGGGCCTATGCCGGAACACTGGGAGGAGG |
| ttvGT1-17... | (400) | CGGGCAGGAGCAGCTGAGCGGAGGGCCTATGCCGGAACACTGGGAGGAGG |
| ttvGT1-21... | (400) | CGGGCAGGAGCAGCTGAGCGGAGGGCCTATGCCGGAACACTGGGAAGAGG |
| ttvgt1-27... | (400) | CGGGCAGGAGCAGCTGAGCGGAGGGCCTATGCCGGAACACTGGGAAGAAG |
| AY823990 | (450) | CCTGGTTGGAAGCTACCAAGGGCTGGCACGATCTCGACTGCCGCTGCGGT |
| ttvg1-7 ... | (450) | CCTGGTTGGAAGCTACCAAGGGCTGGCACGACCTTGACTGCCGCTGCGGT |
| ttvGT1-17... | (450) | CCTGGTTGGAAGCTACCAAGGGCTGGCACGACCTCGACTGCCGCTGCGGT |
| ttvGT1-21... | (450) | CCTGGTTGGAAGCTACCAAGGGCTGGCACGACCTTGACTGCCGCTGCGGT |
| ttvgt1-27... | (450) | CCTGGTTGGAAGCTACCAAGGGCTGGCACGACTTAGACTGCCGCTGCGGT |
| AY823990 | (500) | AACTGGCAGGACCACCTATGGCTCCTACTCGCCGATGGAGACGCCGCTTT |
| ttvg1-7 ... | (500) | AATTGGCAAGACCACCTATGGCTTTTGCTCGCCGATGGAGACGCCGCTTT |
| ttvGT1-17... | (500) | AACTGGCAAGACCACCTATGGCTCCTGCTCGCCGATGGAGACGCGGCTTT |
| ttvGT1-21... | (500) | AATTGGCAAGACCACCTATGGCTTTTGCTCGCCGATGGAGACGCCGCTTT |
| ttvgt1-27... | (500) | AACTGGCAGGACCACCTATGGCTCCTACTCGGCGATGGAGACGCCGCTTT |
| AY823990 | (550) | GGCCGCCGCCGTAGACGCTATAGAAAGAGACGCTATGGCTGGAGACGACG |
| ttvg1-7 ... | (550) | GGCCGCCGCCGTAGACGCTATAGAAAGAGACGCTATGGATGGAGGAGACG |
| ttvGT1-17... | (550) | GGCCGCCGCCGTAGACGCTATAGAAAGAGACGCTGGGGCTGGAGAAGGCG |
| ttvGT1-21... | (550) | GGCCGCCGCCGTAGACGCTATAGAAAGAGACGCTATGGATGGAGGAGACG |
| ttvgt1-27... | (550) | GGCCGCCGCCGTAGACGCTATAGAAAGAGACGCTATGGCTGGAGAAGACG |
| AY823990 | (600) | CTACTACCGCTACAGGCCGCGTGACTATCGGCGACGATGGCTGGTAAGGA |
| ttvg1-7 ... | (600) | CTACTACCGCTACAGACCGCGTTACTATCGGAGACGATGGCTGGTAAGGA |
| ttvGT1-17... | (600) | CTACTGGAGATACCGACCGCGTTACCGTCGGCGCAGATGGCTGGTAAGGA |
| ttvGT1-21... | (600) | CTACTACCGCTACAGACCGCGTTACTATCGGAGACGATGGCTGGTAAGGA |
| ttvgt1-27... | (600) | CTACTACCGCTACAGACCGCGTTACTATCGGAGACGATGGCTGGTAAGGA |
| AY823990 | (650) | GAAGGCGGCGTTCCGTCTACCGTAGAGGTGGACGTAGAGCGCGCCCCTAC |
| ttvg1-7 ... | (650) | GAAGGCGGCGTTCCGTCTACCGACGAGGTGGACGTAGAGCGCGCCCCTAC |
| ttvGT1-17... | (650) | GAAGGCGGCGTTCCGTCTACCGAAGAGGTGGACGTAGAGCGCGCCCCTAC |
| ttvGT1-21... | (650) | GAAGGCGGCGTTCCGTCTACCGACGAGGTGGACGTAGAGCGCGCCCCTAC |
| ttvgt1-27... | (650) | GAAGGCGGCGTTCCGTCTACCGTAGAGGTGGACGTAGAGCGCGCCCCTAC |
| AY823990 | (700) | CGA----CTG--TTTAATCCAAAAGTAATGCGGAGAGTAGTAATTAGGGG |
| ttvg1-7 ... | (700) | CGCATTTCTGCCTTTAATCCGAAAGTAATGCGTAGAGTAGTGATTAGAGG |
| ttvGT1-17... | (700) | CGTATTTCTGCTTTTAATCCAAAAATAATGCGGAGAGTAGTAATAAGGGG |
| ttvGT1-21... | (700) | CGCATTTCTGCCTTTAATCCGAAAGTAATGCGTAGAGTAGTGATTAGAGG |
| ttvgt1-27... | (700) | CGGGTATCTGCCTTTAACCCCAAAGTAATGCGGAGAGTAGTAATAAGGGG |
| AY823990 | (744) | GTGGTGGCCTATTTTACAATGCTTAAAAGGACAGGAGGCACTAAGATATA |
| ttvg1-7 ... | (750) | GTGGTGGCCAATACTGCAGTGCCTAAAAGGTCAGGAATCACTAAGATACA |
| ttvGT1-17... | (750) | ATGGTGGCCAATCCTACAATGTCTAAGAGGACAGGAATCACTAAGATATA |
| ttvGT1-21... | (750) | GTGGTGGCCAATACTGCAGTGCCTAAAAGGTCAGGAATCACTAAGATACA |
| ttvgt1-27... | (750) | GTGGTGGCCAATACTACAGTGCTTAAAAGGACAGGAATCGCTGAGATATA |
| AY823990 | (794) | GACCTCTACAGTGGGACACAGAGAGACAGTGGAGAGTGAGATCAGACTTC |
| ttvg1-7 ... | (800) | GACCACTTCAGTGGGACGTAGAGAAAAGCTGGAGAATAAACACAACTCTT |
| ttvGT1-17... | (800) | GACCGTTACAGTGGGACGTAGAAAAAAGCTGGAGAATAAAGACAGACTTA |
| ttvGT1-21... | (800) | GACCACTTCAGTGGGACGTAGAGAAAAGCTGGAGAATAAACACAACTCTT |
| ttvgt1-27... | (800) | GACCACTACAGTGGGACACAGAAAGACAGTGGAGAGTGAGACAAGACTTC |
| AY823990 | (844) | GAAGACCAGTACGGATACCTCGTACAATACGGGGGAGGTTGGGGAAGTGG |
| ttvg1-7 ... | (850) | GAGGACAACTATGGATACTTAGTACAGTATGGAGGTGGTTGGGGTAGCGG |
| ttvGT1-17... | (850) | GAAGACAACTACGGCTACTTAGTACAGTACGGAGGAGGTTGGGGGAGCGG |
| ttvGT1-21... | (850) | GAGGACAACTATGGATACTTAGTACAGTATGGAGGTGGTTGGGGTAGCGG |
| ttvgt1-27... | (850) | GAGGATCAATACGGATACCTGGTGCAATACGGTGGAGGTTGGGGAAGTGG |
| AY823990 | (894) | TGATGTGACACTTGAAGGTCTCTACCAAGAGCACTTATTGTGGAGAAACT |
| ttvg1-7 ... | (900) | AGAGGTAACACTGGAGGGGCTGTATCAGGAGCACCTACTATGGAGAAACT |
| ttvGT1-17... | (900) | AGAGGTGACTCTAGAAGGACTGTACCAGGAACACCTACTATGGAGAAATT |
| ttvGT1-21... | (900) | AGAGGTAACACTGGAGGGGCTGTATCAGGAGCACCTACTATGGAGAAACT |
| ttvgt1-27... | (900) | TGATGTGACACTAGAGGGACTATACCAGGAACACTTACTATGGAGAAATT |
| AY823990 | (944) | CTTGGTCTAAAGGAAACGATGGAATGGACCTAGTAAGATACTTTGGATGT |
| ttvg1-7 ... | (950) | CTTGGTCAAAAGGAAACGATGGGATGGACTTAGTGAGATACTTCGGCTGC |
| ttvGT1-17... | (950) | CATGGTCAAAAGGAAATGATGGAATGGATCTAGTAAGATACTTCGGCTGC |
| ttvGT1-21... | (950) | CTTGGTCAAAAGGAAACGATGGGATGGACTTAGTGAGATACTTCGGCTGC |
| ttvgt1-27... | (950) | CCTGGTCAAAAGGAAATGATGGCATGGACTTAGTGAGATACTTTGGCTGT |
| AY823990 | (994) | GTAGTATACCTATATCCACTAAAGGACCAGGACTATTGGTTCTGGTGGGA |
| ttvg1-7 ... | (1000) | ATAGTATATCTATATCCGTTAAAAGATCAAGACTACTGGTTTTGGTGGGA |
| ttvGT1-17... | (1000) | ATAGTATACCTGTACCCACTGAAAGATCAGGACTACTGGTTTTGGTGGGA |
| ttvGT1-21... | (1000) | ATAGTATATCTATATCCGTTAAAAGATCAGGACTACTGGTTTTGGTGGGA |
| ttvgt1-27... | (1000) | GTGGTATACCTCTACCCACTTAAAGATCAGGACTATTGGTTCTGGTGGGA |
| AY823990 | (1044) | CACGGACTTCAAAGAATTATATGCAGAAAACATAAAGGAATACAGCCAAC |
| ttvg1-7 ... | (1050) | CACAGATTTTAAAGAATTATATGCAGAGAGTATCAAAGAATACTCACAGC |
| ttvGT1-17... | (1050) | CACAGACTTTAAGGAACTCTATGCAGAAAGTATTAAGGAGTACTCACAAC |
| ttvGT1-21... | (1050) | CACAGATTTTAAGGAATTATATGCAGAGAGTATCAAAGAATACTCACAGC |
| ttvgt1-27... | (1050) | CACTGACTTTAAAGAGCTATACGCAGAAAACATAAAAGAATACAGCCAAC |
| AY823990 | (1094) | CATCAGTAATGATGATGGCAAAAAGAACAAGAATAGTAATAGCCAGAGAA |
| ttvg1-7 ... | (1100) | CATCTGTAATGATGATGGCAAAAAGAACAAAAATAGTGATCGCAAGAAGT |
| ttvGT1-17... | (1100) | CATCAGTAATGATGATGGCAAAAAAAACAAAAATTGTAATAGCGAGAAGT |
| ttvGT1-21... | (1100) | CATCTGTAATGATGATGGCAAAAAGAACAAAAATAGTGATCGCAAGAAGT |
| ttvgt1-27... | (1100) | CATCAGTAATGATGATGGCAAAAAGAACTAGAATAGTAATAGCGAGAGAC |
| AY823990 | (1144) | AGGGCACCACATAGAAGAAAAGTAAGAAAAATATTTATTCCGCCACCTTC |
| ttvg1-7 ... | (1150) | AGAGCCCCACATAGAAGGAAGGTACGCAGAATTTTCATACCGCCTCCAAG |
| ttvGT1-17... | (1150) | AGGGCACCACACAGACGAAAAGTAAGAAAAATATTCATACCGCCACCAAG |
| ttvGT1-21... | (1150) | AGAGCCCCACATAGAAGGAAGGTACGCAGAATTTTCATACCGCCTCCAAG |
| ttvgt1-27... | (1150) | AGAGCTCCACATAGAAGAAAAGTGAGAAAAATATTCATCCCACCACCATC |
| AY823990 | (1194) | GAGAGACACAACACAGTGGCAGTTTCAGACAGATTTCTGCAATAGAAAGT |
| ttvg1-7 ... | (1200) | TAGAGACACGACACAGTGGCAATTTCAAACTGACTTTTGCAATAGACCAC |
| ttvGT1-17... | (1200) | TAGAGACACTACACAATGGCAATTTCAAACAGAGTTCTGCAACAAACCAC |
| ttvGT1-21... | (1200) | TAGAGACACGACACAGTGGCAATTTCAAACTGACTTTTGCAATAGACCAC |
| ttvgt1-27... | (1200) | AAGAGACACTACGCAGTGGCAGTTTCAGACAGACTTCTGTAATAGGAAGC |
| AY823990 | (1244) | TATTTACGTGGGCAGCTGGTCTAATAGACATGCAAAAACCGTTCGATGCT |
| ttvg1-7 ... | (1250) | TATTCACATGGGCTGCAGGACTCATAGACCTCCAAAAACCATTTGACGCA |
| ttvGT1-17... | (1250) | TATTCACTTGGGCTGCAGGACTAATAGACCTCCAAAAGCCATTTGACGCA |
| ttvGT1-21... | (1250) | TATTCACATGGGCTGCAGGACTCATAGACCTCCAAAAACCATTTGACGCA |
| ttvgt1-27... | (1250) | TATTTACCTGGGCGGCAGGACTAATAGACATGCAAAAACCCTTTGATGCC |
| AY823990 | (1294) | AATGGAGCCTTTAGAAATGCTTGGTGGCTGGAACAGAGAAATGATCAGGG |
| ttvg1-7 ... | (1300) | AACGGTGCGTTCAGAAATGCCTGGTGGTTAGAACAGAGAAACGAGGCAGG |
| ttvGT1-17... | (1300) | AACGGAGCTTTTAGAAATGCGTGGTGGTTAGAACAGAGAAATGAGGCAGG |
| ttvGT1-21... | (1300) | AACGGTGCGTTCAGAAATGCCTGGTGGTTAGAACAGAGAAACGAGGCAGG |
| ttvgt1-27... | (1300) | AACGGAGCTTTTAGAAATGCGTGGTGGCTGGAGCAGAGAACGGAACAGGG |
| AY823990 | (1344) | AGAAATGAAATACATAGAACTGTGGGGAAGAGTACCCCCACAAGGAGATT |
| ttvg1-7 ... | (1350) | AGAAATGAAATACATAGAGCTATGGGGTAGAGTACCACCCCAGGGGGACA |
| ttvGT1-17... | (1350) | AGAGATGAAATACATAGAATTATGGGGGAGAGTCCCACCGCAAGGAGACA |
| ttvGT1-21... | (1350) | AGAAATGAAATACATAGAGCTATGGGGTAGAGTACCACCCCAGGGGGACA |
| ttvgt1-27... | (1350) | TGAAATGAAGTACATAGAACTGTGGGGAAGAGTGCCCCCACAAGGAGACT |
| AY823990 | (1394) | CAGAGCTGCCCAAAAAAAAAGAATTCTCCACAGGAACAG---ATAACCCA |
| ttvg1-7 ... | (1400) | CGGAATTACCCGTTCAAACAGAATTCCAAAAACCCTCGGGATATAACCCA |
| ttvGT1-17... | (1400) | CAGAATTGCCGGCCCAAAAAGAATTCCAGAAACCAGACGGGTATAACCCA |
| ttvGT1-21... | (1400) | CGGAATTACCCCTTCAAACAGAATTCCAAAAACCCTCGGGATATAACCCA |
| ttvgt1-27... | (1400) | CAGAACTACCCAAGAAAAGTGAATTCACAACAGCTACAG---ACAATAAA |
| AY823990 | (1441) | AACTACAATGTTCAGGACAATGAGGAGAAAAACATATACCCCATTATAAT |
| ttvg1-7 ... | (1450) | AAATACTACGTAAACCCGGGGGAGGAAAAACCAATCTACCCAGTAATAAT |
| ttvGT1-17... | (1450) | AAATACTATGTGCAGGCAGGAGAGGAAAAACCTATATATCCAATAATAAT |
| ttvGT1-21... | (1450) | AAATACTACGTAAACCCGGGGGAGGAAAAACCAATCTACCCAGTAATAAT |
| ttvgt1-27... | (1447) | AACTACAATGTGAATGACGGTGAGGAAAAACCTATATACCCCATAATTAT |
| AY823990 | (1491) | ATACGTAGACCAAAAAGATCAAAAACCAAGAAAAAAGTACTGCGTATGTT |
| ttvg1-7 ... | (1500) | ATACGTAGACATGAAAGACCAAAAACCAAGAAAAAAGTACTGCGTCTGCT |
| ttvGT1-17... | (1500) | TTACGTAGACAAAAAAGATCAGAAAGCAAGAAAGAAATACTGTGTCTGTT |
| ttvGT1-21... | (1500) | ATACGTAGACATGAAAGACCAAAAACCAAGAAAAAAGTACTGCGTCTGCT |
| ttvgt1-27... | (1497) | ATACGTAGACCAAAAAGACCAAAAACCAAGGAAAAAGTACTGTGTATGTT |
| AY823990 | (1541) | ATAATAAGACCCTCAACAGATGGAGACTAGGACAGGCAAGTACTCTAAAG |
| ttvg1-7 ... | (1550) | ACAACAAGACGCTTAACAGGTGGCGCAGCGCTCAAGCAAGCACATTAAAA |
| ttvGT1-17... | (1550) | ACAATAAGACACTAAACAGATGGAGAGCAGCACAAGCAAGTACCCTAAAA |
| ttvGT1-21... | (1550) | ACAACAAGACGCTTAACAGGTGGCGCAGCGCTCAGGCAAGCACATTAAAA |
| ttvgt1-27... | (1547) | ACAACAAAACTCTGAACAGGTGGAGATTAGGACAAGCGAGTACTCTAAAA |
| AY823990 | (1591) | ATAGGAAACCTGAAAGGACTAGTACTAAGACAGCTGATGAATCAAGAAAT |
| ttvg1-7 ... | (1600) | ATTGGTGACTTGCAGGGGCTAGTATTGAGACAGCTAATGAACCAAGAAAT |
| ttvGT1-17... | (1600) | ATAGGAGACCTGCAAGGACTAGTACTAAGACAATTAATGAACCAGGAAAT |
| ttvGT1-21... | (1600) | ATTGGTGACTTGCAGGGGCTAGTATTGAGACAGCTAATGAACCAAGAAAT |
| ttvgt1-27... | (1597) | ATAGGAAACCTGAAAGGACTAGTGCTAAGACAGTTGATGAACCAAGAGAT |
| AY823990 | (1641) | GACGTATATATGGAAAGAAGGAGAATACAGTGCCCCCTTTGTACAAAGGT |
| ttvg1-7 ... | (1650) | GACATACACATGGAAAGAAGGAGAATTTACCAATGTATTCCTGCAGAGGT |
| ttvGT1-17... | (1650) | GACATATATTTGGAAAGAGGGAGAGTTCACAAACGTATTCCTGCAAAGGT |
| ttvGT1-21... | (1650) | GACATACACATGGAAAGAAGGAGAATTTACAAATGTATTCCTGCAAAGGT |
| ttvgt1-27... | (1647) | GACTTACATATGGAAGGAAGGAGAGTACAGCTCACCATTTGTACAAAGGT |
| AY823990 | (1691) | GGAAAGGCAGCAGATTCGCTGTGATAGACGCAAGAAAGGCAGACCAAGAA |
| ttvg1-7 ... | (1700) | GGAGAGGTTTCAGATTAGCAGTAATAGACGCAAGAAAGGCAGACACAGAA |
| ttvGT1-17... | (1700) | GGAAAGGCTTCAGACTAGCAGTCATAGACGCCAGAAAGGGAGACACAGAA |
| ttvGT1-21... | (1700) | GGAGAGGTTTCAGATTAGCAGTAATAGACGCTAGAAAGGCAGACACAGAA |
| ttvgt1-27... | (1697) | GGAAAGGAAGCAGATTTGTTGTGATAGACGCAAGAAAGGCTGACCAGGAA |
| AY823990 | (1741) | AACCCGAAAGTATCAACATGGCCAATTGAGGGAACGTGGAACACACAGGA |
| ttvg1-7 ... | (1750) | AACCCGACAGTCCAAACTTGGAAGGTGGACGGACAGTGGAACACACAAGG |
| ttvGT1-17... | (1750) | AATCCAACAGTACAAACATGGAAAGTAGACGGAAACTGGAACACTAGTGG |
| ttvGT1-21... | (1750) | AACCCGACAGTCCAAACTTGGAAGGTGGACGGACAGTGGAACACACAAGG |
| ttvgt1-27... | (1747) | AATCCCAAAGTATCTACATGGCCAATAGAGGGAGTGTGGAACACACAGGG |
| AY823990 | (1791) | CACAGTACTGAAGGATGTATTCGGTATTAACTTGCAAAATCAACAATTTA |
| ttvg1-7 ... | (1800) | GACAGTGCTTAAAGAGGTTTTCAATATAAACCTGAATAATGAACAGATGA |
| ttvGT1-17... | (1800) | AACAGTACTACAAGAAGTGTTCGGCATAAACCTCACCCAACAACAAATGA |
| ttvGT1-21... | (1800) | GACAGTTCTTAAAGAGGTTTTCAATATAAACCTGAATAATGAACAGATGA |
| ttvgt1-27... | (1797) | TACAGTACTTAAGGATGTATTCCAGATTGACTTAAACAGTACTAATTTCA |
| AY823990 | (1841) | GGGCGGCGGACTTTGGTAAACTCACACTACCAAAATCACCGCATGACTTA |
| ttvg1-7 ... | (1850) | GACAGGCAGACTTTGGAAAACTAAACTTACCAAAATCCCCGCACGACATT |
| ttvGT1-17... | (1850) | GGGCATCGGACTTTGCTAAGCTAACACTACCAAAATCGCCACATGACATT |
| ttvGT1-21... | (1850) | GACAGGCAGACTTTGGAAAACTAAACTTACCAAAATCCCCGCACGACATT |
| ttvgt1-27... | (1847) | GAGCGGCAGACTTTGGAAAACTAACACTACCAAAATCACCGCACGACTTA |
| AY823990 | (1891) | GACTTCGGTCACCACAGCAGATTTGGGCCATTTTGTGTGAAAAATGAACC |
| ttvg1-7 ... | (1900) | GACTTTGGACACCACAGTAGATTTGGACCTTTCTGTGTAAAAAACGAACC |
| ttvGT1-17... | (1900) | GACTTTGGACACCACAGTAGATTTGGGCCATTTTGTGTCAAAAACGAACC |
| ttvGT1-21... | (1900) | GACTTTGGACACCACAGTAGATTTGGACCTTTCTGTGTAAAAAACGAACC |
| ttvgt1-27... | (1897) | GACTTCGGACATCACAGTAGATTCGGACCATTCTGTGTGAAAAATGAACC |
| AY823990 | (1941) | ACTGGAGTTTCAGGTATACCCTCCAGAACCAACTAACTTGTGGTTTCAGT |
| ttvg1-7 ... | (1950) | ACTGGAGTTTCAACTAACAGCCCCAGAGCCAACTAACCTGTGGTTTCAGT |
| ttvGT1-17... | (1950) | GCTGGAGTTTCAACTAACCGCTCCAGAACCTATTAATCTTTGGTTTCAGT |
| ttvGT1-21... | (1950) | ACTGGAGTTTCAACTAACAGCCCCAGAGCCAACTAACCTGTGGTTTCAGT |
| ttvgt1-27... | (1947) | ACTGGAATTTCAGGTATACCCGCCAGAACCCACTAACCTGTGGTTTCAGT |
| AY823990 | (1991) | ACAGATTTTTCTTTCAGTTTGGAGGTGAATACCAACCCCCCACAGGAATC |
| ttvg1-7 ... | (2000) | ACAAATTTCTGTTTCAGTTTGGAGGTGAATACCAACCACCAACAGGCATC |
| ttvGT1-17... | (2000) | ACAAATTTCTCTTTCAGTTTGGAGGTGAATACCAACCACCAACAGGCATC |
| ttvGT1-21... | (2000) | ACAAATTTCTGTTTCAGTTTGGAGGTGAATACCAACCACCAACAGGCATC |
| ttvgt1-27... | (1997) | ACAGATTTTTCTTTCAGTTTGGAGGTGAATACCAACCCCCCACAGGAATC |
| AY823990 | (2041) | CGGGATCCATGCGTTGATACACCAGCCTATCCTGTGCCGCAGTCAGGAAG |
| ttvg1-7 ... | (2050) | CGCGATCCCTGCGCTGATAACCCAGCCTATCCTGTGCCGCAGTCAGGAAG |
| ttvGT1-17... | (2050) | CGCGATCCCTGCGCTGATAACCAACCCTATCCTGTGCCGCAGTCAGGAAG |
| ttvGT1-21... | (2050) | CGCGATCCCTGCGCTGATAACCCAGCCTATCCTGTGCCGCAGTCAGGAAG |
| ttvgt1-27... | (2047) | CGCGATCCATGCGTTGATACACCAGCCTATCCTGTGCCGCAGTCAGGAAG |
| AY823990 | (2091) | TATTACACACCCCAAATTCGCCGGAAAAGGAGGAATGCTCACGGAAACAG |
| ttvg1-7 ... | (2100) | TATTACACACCCCAAATTCGCCGGAAAAGGCGGCATGCTCACGGAAACAG |
| ttvGT1-17... | (2100) | TATTACACACCCAAAATTCGCCGGGAAAGGAGGAATGCTCACGGAAACAG |
| ttvGT1-21... | (2100) | TATTACACACCCCAAATTCGCCGGAAAAGGCGGCATGCTCACGGAAACAG |
| ttvgt1-27... | (2097) | TATTACACACCCCAAATTCGCCGGAAAAGGCGGAATGCTCACGGAAACAG |
| AY823990 | (2141) | ACCGTTGGGGTATCACTGCTGCCTCTTCCAGAGCCCTCAGTGCAGATACA |
| ttvg1-7 ... | (2150) | ACCGTTGGGGTATCACTGCTGCCTCTTCCCGAACCCTCAGTGCAGATACA |
| ttvGT1-17... | (2150) | ACCGTTGGGGTATCACTGCTGCCTCTTCCAGAGCCCTCAGTGCAGATACA |
| ttvGT1-21... | (2150) | ACCGTTGGGGTATCACTGCTGCCTCTTCCCGAGCCCTCAGTGCAGATACA |
| ttvgt1-27... | (2147) | ACCGTTGGGGTATCACTCCTGCCTCTACCAGAGCCCTCTGTGCAGATACA |
| AY823990 | (2191) | CCCACAGAGGCAGCGCAAAGTGCACTTCTCCGAGGGGACTCGGAAGCGAA |
| ttvg1-7 ... | (2200) | CCCACGGAAGCAACGCAAAGTGCACTTCTCCGAGGGGACTCGGAAAAGAA |
| ttvGT1-17... | (2200) | CCCACGGAGGCAGCGCAAAGTGCACTTCTCCGAGGGGACTCGGAAAAGAA |
| ttvGT1-21... | (2200) | CCCACGGAAGCAACGCAAAGTGCACTTCTCCGAGGGGACTCGGAAAAGAA |
| ttvgt1-27... | (2197) | CCCACAGAAGCAACGCAGAGTGCACTTCTCCGAGGGGACTCGGAAAAGAA |
| AY823990 | (2241) | AGGAGAGGAAACCGAGGAAACCGCGTCATCGTCCAGTATCACGAGTGCCG |
| ttvg1-7 ... | (2250) | AGGAGAGGAAACCGAGGAAACCTCGTCATCGTCCAGTATCACGAGTGCCG |
| ttvGT1-17... | (2250) | AGGAGAGGAAACCGAGGAAACCACGTCATCGTCCAGTATCACGAGTGCCG |
| ttvGT1-21... | (2250) | AGGAGAGGAAACCGAGGAAACCTCGTCATCGTCCAGTATCACGAGTGCCG |
| ttvgt1-27... | (2247) | AGGAGAGGAAACCGAGGAAACCACGTCATCGTCCAGTATCACGAGTGCCG |
| AY823990 | (2291) | AAAGCTCTACTGAGGGAGATGGATCGTCTGATGATGAAGAGACAATCAGA |
| ttvg1-7 ... | (2300) | AAAGCTCTACTGAAGGAGATGGATCGTCTGATGATGAAGAGACAATCAGA |
| ttvGT1-17... | (2300) | AAAGCTCTACTGAAGGAGATGGATCGTCTGATGATGAAGAGACAATCCGA |
| ttvGT1-21... | (2300) | AAAGCTCTACTGAAGGAGATGGATCGTCTGATGATGAAGAGACAATCAGA |
| ttvgt1-27... | (2297) | AAAGCTCTACTGAGGGAGATGGATCGTCTGATGATGAAGAGACAGTCAGA |
| AY823990 | (2341) | CGCAGAAGGAGGACCTGGAAGCGACTCAGACGAATGGTCAGAGAGCAGCT |
| ttvg1-7 ... | (2350) | CGCCGAAGGAGGACCTGGAAGCGACTCAGACGGATGGTCCGAGAGCAGCT |
| ttvGT1-17... | (2350) | CGCAGAAGGAGGACCTGGAAGCGACTCCGACGAATGGTCAGAGAGCAGCT |
| ttvGT1-21... | (2350) | CGCCGAAGGAGGACCTGGAAGCGACTCAGACGGATGGTCCGAGAGCAGCT |
| ttvgt1-27... | (2347) | CGCCGAAGGAGGACCTGGAAGCGACTCAGACGAATGGTCCGAGAGCAGCT |
| AY823990 | (2391) | TGACCGACGAATGGACCACAAGCGACAGCGACTTCATTGACACCCCCATA |
| ttvg1-7 ... | (2400) | TGACCGACGAATGGACCACAAGCGACAGCGACTTCATTGACACCCCCATT |
| ttvGT1-17... | (2400) | TGACCGACGAATGGACCACAAGCGACAGCGACTTCATTGACACCCCCATA |
| ttvGT1-21... | (2400) | TGACCGACGAATGGACCACAAGCGACAGCGACTTCATTGACACCCCCATT |
| ttvgt1-27... | (2397) | TGACCGACGAATGGACCACAAGCGACAGCGACTTCATTGACACCCCCATT |
| AY823990 | (2441) | AGAGAAAGATGCCTCAATAAAAAACAAAAGAAACGCTAAACAGTGTCCGA |
| ttvg1-7 ... | (2450) | AAACAGAGATGCCTCAATAAAAAACAAAAGAAACGCTAAGCAGTGTCC-C |
| ttvGT1-17... | (2450) | AGAGAACGATGCCTGAATAAAAAACAAAAAAAACGCTACACAGTGTCCGC |
| ttvGT1-21... | (2450) | AGACAGAGATGCCTCAATAAAAAGCAAAAGAAACGCTAAACAGTGTCC-C |
| ttvgt1-27... | (2447) | AGAGACAGATGCCTCAATAAAAAGCAAAAGAAACGCTAAACTGCCTCCGC |
| AY823990 | (2491) | TTACTAATGGGGGGGGGTCCGGGGGGGGCTTGCCCCCCCGCAAGCTGGGT |
| ttvg1-7 ... | (2499) | TATTATTTTGGGGGG--TCCGGGGGGGGCTTGCCCCCCCGTAAGCTGGGT |
| ttvGT1-17... | (2500) | TTATTTGTAGGGGGGG-TCCGGGGGGGGCTTGCCCCCCCGTAAGCTGGGT |
| ttvGT1-21... | (2499) | TATTACTTTGGGGGGG-TCCGGGGGGGGCTTGCCCCCCCGTAAGCTGTGT |
| ttvgt1-27... | (2497) | TTATTTTTTGGGGGG--TCCGGGGGGGGCTTGCCCCCCCGAAAGCTGGGT |
| AY823990 | (2541) | TACCGCACTAACTCCCTGCCAAGTGAAACTCGGGGACGAGTGAGTGCGGG |
| ttvg1-7 ... | (2547) | TACCGCACTAACTCCCTGCCAAGTGAAACTCGGGGACGAGTGAGTGCGGG |
| ttvGT1-17... | (2549) | TGCCGCACTAACTCCCTGCCAAGTGAAACTCGGGGACGAGTGAGTGCGGG |
| ttvGT1-21... | (2548) | TACCGCACTAACTCCCTGCCAAGTGAAACTCGGGGACGAGTGAGTGCGGG |
| ttvgt1-27... | (2545) | TACCGCACTAACTCCCTGCCAAGTGAAACTCGGGGACGAGTGAGTGCGGG |
| AY823990 | (2591) | ACATCCCGTGTAATGGCTACATAACTACCCGGCTTTGCTTCGACAGTGGC |
| ttvg1-7 ... | (2597) | ACATCCCGTGTAATGGCTACATAACTACCCGGCTTTGCTTCGACAGTGGC |
| ttvGT1-17... | (2599) | ACATCCCGTGTAATGGCTACATAACTACCCGGCTTTGCTTCGACAGTGGC |
| ttvGT1-21... | (2598) | ACATCCCGTGTAATGGCTACATAACTACCCGGCTTTGCTTCCACAGTGGC |
| ttvgt1-27... | (2595) | ACATCCCGTGTAATGGCTACATAACTACCCGGCTTTGCTTCGACAGTGGC |
| AY823990 | (2641) | CGTGGCTCGACCCTCACACAACACTGCAGGTAGGGGGCGCAATTGGGATC |
| ttvg1-7 ... | (2647) | CGTGGCTCGACCCTCACACAACACTGCAGGTAGGGGGCGCAATTGTGATC |
| ttvGT1-17... | (2649) | CGTGGCTCGACCCTCACACAACAATGCAGGTAGGGGGCGCAATTGGGATC |
| ttvGT1-21... | (2648) | CGTGGCTCGACCCTCACACAACACTGCAGGTAGGGGGCGCAATTGGGATC |
| ttvgt1-27... | (2645) | CGTGGCTCGACCCTCACACAACACTGCAGATAGGGGGCGCAATTGGGATC |
| AY823990 | (2691) | GTTAGAAAACTATGGCC--GAGCATGGGGGNNNNNNNNNNNNNNCCAACC |
| ttvg1-7 ... | (2697) | GTTAGAAAACTATGGCCCGGAGCATGG-CCCCCCAAAC------CCCCCC |
| ttvGT1-17... | (2699) | GTTAGAAAACTATGGCCCG-AGCATGGGCCCCCCAAAA------CCCCCC |
| ttvGT1-21... | (2698) | GTTAGAAAACTATGGCCCCAAGCATGG-CCCA--AAAC------CCCCCC |
| ttvgt1-27... | (2695) | GTTAGAAAACTATGGCC--GAGCATGGGCCCCCACAAA-----CCCCCCC |
| AY823990 | (2739) | CCCCCGGTGGGGGGGCCAAGGCCCCCCCTACACCCCCCCATGGGGGGCTG |
| ttvg1-7 ... | (2740) | TTGCCCGGGGCTGTGCCCCGGACCCCC----------------------- |
| ttvGT1-17... | (2742) | TTGCCCGGGGCTGTGCCCCGGACCCCC----------------------- |
| ttvGT1-21... | (2739) | TT-CCCGGGGCTGTGCCCCGGACCCCC----------------------- |
| ttvgt1-27... | (2738) | CTGCCCGGGGCTGTGCCCCGGACCCCCC---------------------- |
| AY823990 | (2789) | CCGCCCCCCAAACCCCCCGCGTCGGATGGGGGGGGCTGCGCCCCCCCCAA |
| ttvg1-7 ... | (2767) | -------------------------------------------------- |
| ttvGT1-17... | (2769) | -------------------------------------------------- |
| ttvGT1-21... | (2765) | -------------------------------------------------- |
| ttvgt1-27... | (2766) | -------------------------------------------------- |
| AY823990 | (2839) | ACCCCCCTTGCCCGGGGCTGTGCCCCGGACCCCC |
| ttvg1-7 ... | (2767) | ---------------------------------- |
| ttvGT1-17... | (2769) | ---------------------------------- |
| ttvGT1-21... | (2765) | ---------------------------------- |
| ttvgt1-27... | (2766) | ---------------------------------- |

### Nucleotide Identity Among PAH TTV's and Published Sequence

| | AY823990 | ttvg1-7 | ttvGT1-17 | ttvGT1-21 | ttvgt1-27 |
|---|---|---|---|---|---|
| AY823990 | | 85 | 87 | 85 | 91 |
| ttvg1-7 | | | 89 | 99 | 86 |
| ttvGT1-17 | | | | 89 | 86 |
| ttvGT1-21 | | | | | 86 |
| ttvgt1-27 | | | | | |

TTVgt1-27 demonstrates the greatest homology with published sequence, AY823990, demonstrating 91% identity. TTVgt1-7,17, and 21 demonstrate 85-87% identity. TTVgt1-7 and TTVgt1-21 share 99% nucleotide identity

### Orf1 Amino Acid Alignment

The following provides a comparison of the published AY823990 sequence (SEQ ID NO:25) to the corresponding amino acid sequences for TTV7 (SEQ ID NO:10), TTV17 (SEQ ID NO:11), TTV21 (SEQ ID NO:12), and TTV27 (SEQ ID NO:13)

| | | |
|---|---|---|
| AY823990 | (1) | MAPTRRWRRRFGRRRRRYRKRRYGWRRRYYRYRPRDYRRRWLVRRRRRSV |
| Ttvg1-7Orf1 | (1) | MAFARRWRRRFGRRRRRYRKRRYGWRRRYYRYRPRYYRRRWLVRRRRRSV |
| Ttg1-17Orf1 | (1) | MAPARRWRRGFGRRRRRYRKRRWGWRRRYWRYRPRYRRRRWVVRRRRRSV |
| Ttg1-27Orf1 | (1) | MAPTRRWRRRFGRRRRRYRKRRYGWRRRYYRYRPRYYRRRWLVRRRRRSV |
| ttg1-21Orf1 | (1) | MAFARRWRRRFGRRRRRYRKRRYGWRRRYYRYRPRYYRRRWLVRRRRRSV |
| AY823990 | (51) | YRRGGRRARPYRL--FNPKVMRRVVIRGWWPILQCLKGQEALRYRPLQWD |
| Ttvg1-7Orf1 | (51) | YRRGGRRARPYRISAFNPKVMRRVVIRGWWPILQCLKGQESLRYRPLQWD |
| Ttg1-17Orf1 | (51) | YRRGGRRARPYRISAFNPKIMRRVVIRGWWPILQCLRGQESLRYRPLQWD |
| Ttg1-27Orf1 | (51) | YRRGGRRARPYRVSAFNPKVMRRVVIRGWWPILQCLKGQESLRYRPLQWD |
| ttg1-21Orf1 | (51) | YRRGGRRARPYRISAFNPKVMRRVVIRGWWPILQCLKGQESLRYRPLQWD |
| AY823990 | (99) | TERQWRVRSDFEDQYGYLVQYGGGWGSGDVTLEGLYQEHLLWRNSWSKGN |
| Ttvg1-7Orf1 | (101) | VEKSWRINTTLEDNYGYLVQYGGGWGSGEVTLEGLYQEHLLWRNSWSKGN |
| Ttg1-17Orf1 | (101) | VEKSWRIKTDLEDNYGYLVQYGGGWGSGEVTLEGLYQEHLLWRNSWSKGN |
| Ttg1-27Orf1 | (101) | TERQWRVRQDFEDQYGYLVQYGGGWGSGDVTLEGLYQEHLLWRNSWSKGN |
| ttg1-21Orf1 | (101) | VEKSWRINTTLEDNYGYLVQYGGGWGSGEVTLEGLYQEHLLWRNSWSKGN |
| AY823990 | (149) | DGMDLVRYFGCVVYLYPLKDQDYWFWWDTDFKELYAENIKEYSQPSVMMM |
| Ttvg1-7Orf1 | (151) | DGMDLVRYFGCIVYLYPLKDQDYWFWWDTDFKELYAESIKEYSQPSVMMM |
| Ttg1-17Orf1 | (151) | DGMDLVRYFGCIVYLYPLKDQDYWFWWDTDFKELYAESIKEYSQPSVMMM |
| Ttg1-27Orf1 | (151) | DGMDLVRYFGCVVYLYPLKDQDYWFWWDTDFKELYAENIKEYSQPSVMMM |
| ttg1-21Orf1 | (151) | DGMDLVRYFGCIVYLYPLKDQDYWFWWDTDFKELYAESIKEYSQPSVMMM |
| AY823990 | (199) | AKRTRIVIARERAPHRRKVRKIFIPPPSRDTTQWQFQTDFCNRKLFTWAA |
| Ttvg1-7Orf1 | (201) | AKRTKIVIARSRAPHRRKVRRIFIPPPSRDTTQWQFQTDFCNRPLFTWAA |
| Ttg1-17Orf1 | (201) | AKKTKIVIARSRAPHRRKVRKIFIPPPSRDTTQWQFQTEFCNKPLFTWAA |
| Ttg1-27Orf1 | (201) | AKRTRIVIARDRAPHRRKVRKIFIPPPSRDTTQWQFQTDFCNRKLFTWAA |
| ttg1-21Orf1 | (201) | AKRTKIVIARSRAPHRRKVRRIFIPPPSRDTTQWQFQTDFCNRPLFTWAA |
| AY823990 | (249) | GLIDMQKPFDANGAFRNAWWLEQRNDQGEMKYIELWGRVPPQGDSELPKK |
| Ttvg1-7Orf1 | (251) | GLIDLQKPFDANGAFRNAWWLEQRNEAGEMKYIELWGRVPPQGDTELPVQ |
| Ttg1-17Orf1 | (251) | GLIDLQKPFDANGAFRNAWWLEQRNEAGEMKYIELWGRVPPQGDTELPAQ |
| Ttg1-27Orf1 | (251) | GLIDMQKPFDANGAFRNAWWLEQRTEQGEMKYIELWGRVPPQGDSELPKK |
| ttg1-21Orf1 | (251) | GLIDLQKPFDANGAFRNAWWLEQRNEAGEMKYIELWGRVPPQGDTELPLQ |
| AY823990 | (299) | KEFSTGT-DNPNYNVQDNEEKNIYPIIIYVDQKDQKPRKKYCVCYNKTLN |
| Ttvg1-7Orf1 | (301) | TEFQKPSGYNPKYYVNPGEEKPIYPVIIYVDMKDQKPRKKYCVCYNKTLN |
| Ttg1-17Orf1 | (301) | KEFQKPDGYNPKYYVQAGEEKPIYPIIIYVDKKDQKARKKYCVCYNKTLN |
| Ttg1-27Orf1 | (301) | SEFTTAT-DNKNYNVNDGEEKPIYPIIIYVDQKDQKPRKKYCVCYNKTLN |
| ttg1-21Orf1 | (301) | TEFQKPSGYNPKYYVNPGEEKPIYPVIIYVDMKDQKPRKKYCVCYNKTLN |
| AY823990 | (348) | RWRLGQASTLKIGNLKGLVLRQLMNQEMTYIWKEGEYSAPFVQRWKGSRF |
| Ttvg1-7Orf1 | (351) | RWRSAQASTLKIGDLQGLVLRQLMNQEMTYTWKEGEFTNVFLQRWRGFRL |
| Ttg1-17Orf1 | (351) | RWRAAQASTLKIGDLQGLVLRQLMNQEMTYIWKEGEFTNVFLQRWKGERL |
| Ttg1-27Orf1 | (350) | RWRLGQASTLKIGNLKGLVLRQLMNQEMTYIWKEGEYSSPFVQRWKGSRF |
| ttg1-21Orf1 | (351) | RWRSAQASTLKIGDLQGLVLRQLMNQEMTYTWKEGEFTNVFLQRWRGFRL |
| AY823990 | (398) | AVIDARKADQENPKVSTWPIEGTWNTQDTVLKDVFGINLQNQQFRAADFG |
| Ttvg1-7Orf1 | (401) | AVIDARKADTENPTVQTWKVDGQWNTQGTVLKEVFNINLNNEQMRQADFG |
| Ttg1-17Orf1 | (401) | AVIDARKGDTENPTVQTWKVDGNWNTSGTVLQEVFGINLTQQQMRASDFA |
| Ttg1-27Orf1 | (400) | VVIDARKADQENPKVSTWPIEGVWNTQGTVLKDVFQIDLNSTNFRAADFG |
| ttg1-21Orf1 | (401) | AVIDARKADTENPTVQTWKVDGQWNTQGTVLKEVFNINLNNEQMRQADFG |
| AY823990 | (448) | KLTLPKSPHDLDFGHHSRFGPFCVKNEPLEFQVYPPEPTNLWFQYRFFFQ |
| Ttvg1-7Orf1 | (451) | KLNLPKSPHDIDFGHHSRFGPFCVKNEPLEFQLTAPEPTNLWFQYKFLFQ |
| Ttg1-17Orf1 | (451) | KLTLPKSPHDIDFGHHSRFGPFCVKNEPLEFQLTAPEPINLWFQYKFLFQ |
| Ttg1-27Orf1 | (450) | KLTLPKSPHDLDFGHHSRFGPFCVKNEPLEFQVYPPEPTNLWFQYRFFFQ |
| ttg1-21Orf1 | (451) | KLNLPKSPHDIDFGHHSRFGPFCVKNEPLEFQLTAPEPTNLWFQYKFLFQ |
| AY823990 | (498) | FGGEYQPPTGIRDPCVDTPAYPVPQSGSITHPKFAGKGGMLTETDRWGIT |
| Ttvg1-7Orf1 | (501) | FGGEYQPPTGIRDPCADNPAYPVPQSGSITHPKFAGKGGMLTETDRWGIT |
| Ttg1-17Orf1 | (501) | FGGEYQPPTGIRDPCADNQPYPVPQSGSITHPKFAGKGGMLTETDRWGIT |
| Ttg1-27Orf1 | (500) | FGGEYQPPTGIRDPCVDTPAYPVPQSGSITHPKFAGKGGMLTETDRWGIT |
| ttg1-21Orf1 | (501) | FGGEYQPPTGIRDPCADNPAYPVPQSGSITHPKFAGKGGMLTETDRWGIT |
| AY823990 | (548) | AASSRALSADTPTEAAQSALLRGDSEAKGEETEETASSSSITSAESSTEG |
| Ttvg1-7Orf1 | (551) | AASSRTLSADTPTEATQSALLRGDSEKKGEETEETSSSSSITSAESSTEG |
| Ttg1-17Orf1 | (551) | AASSRALSADTPTEAAQSALLRGDSEKKGEETEETTSSSSITSAESSTEG |
| Ttg1-27Orf1 | (550) | PASTRALCADTPTEATQSALLRGDSEKKGEETEETTSSSSITSAESSTEG |
| ttg1-21Orf1 | (551) | AASSRALSADTPTEATQSALLRGDSEKKGEETEETSSSSSITSAESSTEG |
| AY823990 | (598) | DGSSDDEETIRRRRRTWKRLRRMVREQLDRRMDHKRQRLH- |
| Ttvg1-7Orf1 | (601) | DGSSDDEETIRRRRRTWKRLRRMVREQLDRRMDHKRQRLH- |
| Ttg1-17Orf1 | (601) | DGSSDDEETIRRRRRTWKRLRRMVREQLDRRMDHKRQRLH- |
| Ttg1-27Orf1 | (600) | DGSSDDEETVRRRRRTWKRLRRMVREQLDRRMDHKRQRLH- |
| ttg1-21Orf1 | (601) | DGSSDDEETIRRRRRTWKRLRRMVREQLDRRMDHKRQRLH- |

| | | ttvg1- | ttvgt1- | ttvgt1- | ttvg1- |
|---|---|---|---|---|---|
| | AY823990ORF1 | 7ORF1 | 16ORF1 | 27ORF1 | 21ORF1 |
| Ay823990ORF1 | | 87 | 86 | 95 | 87 |
| ttvg1-7ORF1 | | | 93 | 87 | 100 |
| ttvgt1-17ORF1 | | | | 85 | 93 |
| ttvgt1-27ORF1 | | | | | 87 |
| ttvg1-21ORF1 | | | | | |

Hydrophobicity plots of the proteins demonstrate 5 areas of hydrophilicity, which may indicate surface-exposed regions that are potentially antigenic. Two of these regions are at the amino terminus and at the carboxy terminus, and are both arginine-rich and highly conserved. A highly conserved hydrophilic region between amino acids 190 and 232 was observed and may potentially serve as antigenic site. The remaining hydrophilic regions between amino acids 295 and 316, and between amino acids 415 and 470 are also be antigenic.

Additionally, it has been determined that the putative start codons for ORF1 and coding region are as follows: ttvgt1-27 nt 517-2435; ttvg1-7 nt 517-2435; ttvgt1-17 nt 517-2436; ttvgt1-21 nt 517-2439; ttv10 nt 487-2346; and ttv13 nt 477-2363. The putative start codons for ORF 2 and coding region are as follows: ttvgt1-27 nt 428-646; ttvg1-7 nt 428-643; ttvgt1-17 nt 428-643; ttvgt1-21 nt 428-646; ttv10 nt 404-610; and ttv13 nt 394-597.

### TTV ORF1 Protein Expression Utilizing Recombinant Baculovirus

A series of experiments was then undertaken to express the genotype 2 TTV ORF1 protein utilizing insect cells and recombinant baculovirus. Optimization of protein expression was conducted with three cell lines (SF9, SF21 and Hi Five), multiple media configurations (ExCell 420, SF900 III SFM, Express Five SFM), various cell densities (5e5, 1e6, 2e6 and 4e6 cells/ml), and various multiplicities of infection (0.005, 0.1, 0.5, 2.0), and the resultant cultures were monitored daily over a seven day post infection period.

The processes were monitored for cell density and viability, and infection was monitored through monitoring of cell size and virus titration. Protein expression was monitored through SDS-PAGE, Coomassie gel analysis and Western blotting. To ensure proper control, negative and positive controls were maintained throughout all experiments. Although all experiments were able to confirm expression of the target protein, optimal conditions were found when utilizing SF9 cells maintained in ExCell media (Sigma, SAFC) with a cell density of 2×10⁶ cells/ml and an MOI of 0.1, with the process terminated following a three day infection. The majority of the recombinant expressed protein can be located within the cell pellet although some resides in the resultant supernatant.

Confirmation of Protein Expression with Western Blotting (GST-Tag)

As the Invitrogen destination vectors (pDEST10) contained a GST protein N-terminal to the TTV Orf1 reading frame, a resultant GST-ORF1 fusion protein of approximately 95 kD was generated, which was detected using a commercially available rabbit anti-GST (CALBIOCHEM) antibody. Of the 95 kD fusion protein, approximately 68 kD is considered to be ORF1 and 25 kD to be the GST protein. No commercial antibody was available for standardized detection of TTV ORF1 protein, which necessitated the use of the anti-GST antibody.

### Production of Rabbit Anti-TTV ORF1 Antibody

Due to the initial lack of availability of known TTV reagents, efforts were undertaken to produce anti-TTV ORF1 antibodies. Following the optimized expression protocol for preparing the TTV ORF1 recombinant protein, the resultant material was further purified utilizing the commercially available Baculogold GST purification kit. Purified TTV10 and TTV13 ORF1 protein was then utilized to hyperimmunize rabbits for the subsequent production of antibodies against the ORF1 recombinant protein.

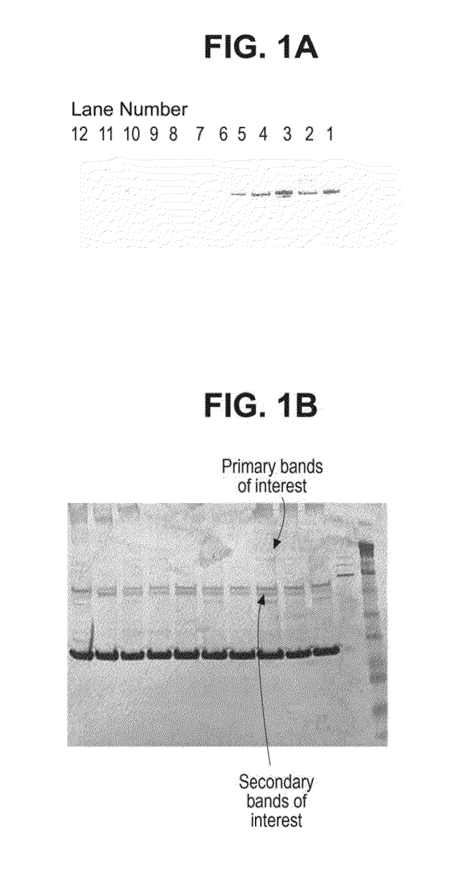

In regard of protein detection, FIG. 1A sample lanes were as follows (from right to left)

| Samples: | |
|---|---|
| 1 | See Blue Plus 2 |
| 2 | ORF1 TTV13 d.3 1e6 cell/ml (GST purified pellet) |
| 3 | ORF1 TTV13 d.3 1e6 cell/ml (GST unbound) |
| 4 | ORF1 TTV13 d.3 2e6 cell/ml (GST purified pellet) |
| 5 | ORF1 TTV13 d.3 2e6 cell/ml (GST unbound) |
| 6 | ORF1 TTV13 d.3 4e6 cell/ml (GST purified pellet) |
| 7 | ORF1 TTV13 d.3 4e6 cell/ml (GST unbound) |
| 8 | ORF1 TTV13 d.3 4e6 cell/ml (GST purified supe) |
| 9 | ORF1 TTV13 d.3 4e6 cell/ml (GST unbound) |
| 10 | ORF1 TTV13 d.3 4e6 cell/ml untreated supe.) |
| 11 | ORF1 TTV13 d.3 4e6 cell/ml untreated cell pellet |
| 12 | SF9 Negative Control d.3 pellet 1e6 cell/ml |

Lanes 2, 4 and 6 demonstrate the purified 95 kD TTV13 ORF1 fusion protein which was later utilized for the rabbit immunization, see FIG. 1A.

### Detection of Native TTV ORF1 Utilizing the Rabbit Anti-ORF1 Protein

Additional expression experiments were conducted with the native TTV ORF1 recombinant baculovirus. This recombinant baculovirus was constructed without a 6xHis or GST fusion tag and hence requires a specific anti-TTV ORF1 antibody. Consequently, post expression Western blot analysis was conducted utilizing the rabbit anti-TTV ORF1 antibody to confirm expression of the native protein, and to confirm the reagent reactivity. Western blot analysis demonstrated a faint reaction at approximately 69 kD, which is approximately the predicted size of TTV ORF1 as well as reaction to an additional band at approximately 49 kD (see FIG. 1B). The 49 kD protein band is unknown. The faint banding at 69 kD is assumed to be a function of either low protein expression in the native TTV ORF1 construct or poor antibody yield from the rabbit immunization. It should be noted that no purification of the antigen or antibody was conducted in this particular analysis. Lane 5 (see the arrows in FIG. 1B) demonstrates a unique reaction to a 69 kD and 49 kD protein in the native TTV ORF1 expression utilizing anti-TTV ORF1 rabbit polyclonal antibody.

Accordingly, there was demonstrated binding of antibody to capsid protein as antigen, herein the antigen provided only TTV sequence and was not tagged.

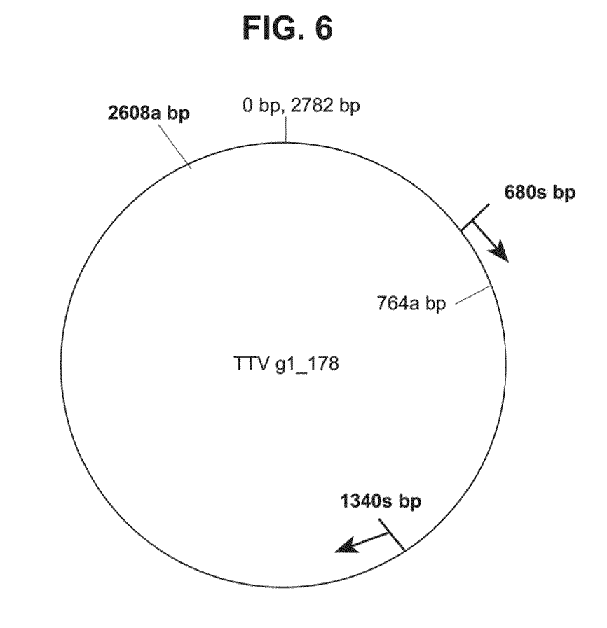

FIG. 6B Sample Lanes were as Follows (from Right to Left)

| Samples: | |
|---|---|
| 1 | SeeBlue Plus 2 |
| 2 | g1TTV standard 1:50 diluted |
| 3 | g2TTV13 ORF1 Native 2DPI cell/supe 0.005 MOI |
| 4 | g2TTV13 ORF1 Native 3DPI cell/supe 0.005 MOI |
| 5 | g2TTV13 ORF1 Native 4DPI cell/supe 0.005 MOI |
| 6 | g2TTV13 ORF1 Native 2DPI cell/supe 0.1 MOI |
| 7 | g2TTV13 ORF1 Native 3DPI cell/supe 0.1 MOI |
| 8 | g2TTV13 ORF1 Native 4DPI cell/supe 0.1 MOI |
| 9 | g2TTV13 ORF1 Native 2DPI cell/supe 2.0 MOI |
| 10 | g2TTV13 ORF1 Native 3DPI cell/supe 2.0 MOI |
| 11 | g2TTV13 ORF1 Native 4DPI cell/supe 2.0 MOI |
| 12 | SF9 Neg. Control 4DPP cell/supe |

### Example 2

### Backpassaging

A liver was collected aseptically from a caesarean-derived, colostrum deprived (CDCD) pig. The liver tissue was tested for g1 and g2 TTV in unique qPCR assays and confirmed to be positive for only g1TTV. A 10% (wt/vol) liver homogenate was then prepared in media containing antibiotics and antimycotics. Finally, the homogenate was clarified by centrifugation, designated as g1TTVp0 and frozen at −70 C. The resulting g1TTV homogenate was tested to be free of extraneous viruses, bacteria and mycoplasma via routine testing. Following satisfactory testing, two milliliters of freshly thawed g1TTVp0 was IP inoculated into each of six 11-day old gnotobiotic piglets. At approximately 12 days post-inoculation the pigs were euthanized and the bone marrow, spleen and livers were aseptically collected. Each of the resulting livers were confirmed by qPCR to be rich in g1TTV and negative for g2TTV. Liver homogenates were then prepared from each of the resulting livers as aforementioned, labeled and aliquoted as g1TTVp1 and placed at −70 C. A further second passage (g1TTVp2) was created from g1TTVp1.

### Example 3

### Evaluation of the Efficacy of Three Torque Teno Virus (TTV) Vaccines in Young Pigs

The present study was conducted to evaluate the efficacy of three TTV vaccine candidates administered at ˜7 days of age, and again at weaning (˜21 days of age) followed by a challenge at ˜5 weeks of age.

This study provided a preliminary immunogenicity evaluation in pigs injected intramuscularly with formulations for TTV. As previously mentioned, TTV is a small, non-enveloped virus with a single-stranded circular DNA genome of negative polarity. The genome includes an untranslated region and at least three major overlapping open reading frames. Porcine TTV is ubiquitous and PCR-detection of the virus in serum samples collected from various geographical regions shows prevalence in pigs ranging from 33 to 100%. McKeown et al., Vet. Microbiol. (2004) 104:113-117. Krakowka et al., AJVR (2008) 69: 1623-1629, reported that g1-TTV inoculated pigs had no clinical signs but developed interstitial pneumonia, transient thymic atrophy, membranous glomerulonephropathy and modest lymphocytic to histiocytic infiltrates in the liver after inoculation. The present study provided a comparison of three different formulations of TTV vaccines, and evaluated if any of these prototype formulations can be numerically or statistically differentiated when compared to challenge control groups.

### Materials and Methods

Animals: Six clinically healthy, crossbred pregnant, PRRSV and M hyo seronegative females without a history of disease caused by PRRSV or M hyo (or vaccination against the same organisms) were sourced from Lincoln Trail/Puregenic Pork, Alton, Ill., and transported to the Pfizer Animal Health Research Farm in Richland, Mich. at approximately 3 weeks pre-partum. If necessary, sows were induced to farrow within a 2 or 3 day period using injectable prostaglandin (Lutalyse®). Normal piglets from these sows were allotted to study according to the allotment design. Pigs were randomized to treatment by litter and each litter had at least one piglet assigned to each treatment.

Housing: During the vaccination phase, pigs were housed with their mother with no cross-fostering, in BL-2 isolation facilities. Pigs remained housed by litter until the time of 2^{nd} vaccination. Post-second vaccination pigs were moved to a further facility and housed in two rooms (one room contains NTX (non-vaccinated and non-challenge controls) animals, the second room vaccinates), and each room contains or 8 pigs per pen.

Feed: Following farrowing, sows were fed a lactating sow diet as appropriate. Piglets accessed creep feed and milk replacer prior to weaning. Once weaned, piglets were feed an age-appropriate diet offering free choice. Water was available to all animals ad libitum.

Allotment/Randomization: Pigs were randomized to treatment by litter. Each litter had at least one piglet assigned to each treatment.

### Study Design

| | | Dose/ | | Challenge/ | # of |
|---|---|---|---|---|---|
| TX | Inoculum | Route | Vacc. Days | Route | Pigs |
| NTX* | NA | NA | NA | NA | ~10 |
| T01 | Chromos g1TTV | IM | ~7 days of | g1TTV pass1/IP | ~10 |
| | ORF1 recombinant | | age and at | | |
| | protein | | weaning | | |
| T02 | Baculovirus g2TTV | IM | (Day 21) | | ~10 |
| | ORF1 recombinant | | | | |
| | protein | | | | |
| T03 | Inactivated challenge | IM | | | ~10 |
| | virus g1TTVp1. | | | | |
| T04 | Mock | IM | | | ~10 |

*Minimum of 1 NTX pig from each litter

Masking: Vaccine was masked using a numeric code prior to vaccination. The investigator, vaccine administrator and study personnel were masked to treatment and did not have access to the masking code unless treatment information was required for the welfare of an animal.

### Investigational Veterinary Products

TABLE 1
IVP Formulation

| | | |
|---|---|---|
| T01 | True Name: | Chromos g1TTV ORF1 recombinant protein |
| | Serial Number: | # 117473-185C |
| | Dosage/Formulation: | 2 mL; formulated to contain equal volumes |
| | | of g1TTV ORF1 recombinant protein and |
| | | sterile 5% Amphigen diluent. |
| T02 | True Name: | Baculovirus g2TTV ORF1 recombinant |
| | | protein killed subunit vaccine |
| | Serial Number: | # 117473-185B |
| | Dosage/Formulation: | 2 mL; formulated to contain equal volumes |
| | | of g2TTV ORF1 recombinant protein and |
| | | sterile 5% Amphigen diluent. |
| T03 | True Name: | Torque Teno Vaccine, g1TTVp1 Killed Virus |
| | Serial Number: | # 117473-185D |
| | Dosage/Formulation: | 2 mL; formulated to contain equal volumes |
| | | of g1TTVp1 KV antigen and sterile 5% |
| | | Amphigen diluent. |
| T04 | True Name: | Mock (Placebo) |
| | Serial Number: | # 117473-185A |
| | Dosage/Formulation: | 2 mL; formulated to contain equal volumes of |
| | | Phosphate buffered saline and sterile 5% |
| | | Amphigen diluent. |

Challenge Material Preparation: g1TTV pass 1 was derived from liver homogenate tested positive (7.6×10e8 to 1.6×10e9 DNA copies/2 mL) for g1TTV and negative for g2TTV by qPCR. An appropriate number of bottles were removed from the freezer and thawed shortly before challenge. An aliquot was then removed from one of the bottles, and held for retitration at a later time. Challenge stock was transported on ice to the research facility and maintained on ice during the challenge procedure. A challenge dose equals 2.0 mL of stock solution (2.0 mL intraperitoneal). The dose was delivered to each pig is therefore expected to be 7.6×10e8 to 1.6×10e9 DNA copies/2 mL. Following challenge, an aliquot of challenge stock was kept for titration to confirm challenge dose.

General Health Observations: Animals were observed daily by a qualified individual and general health observations were recorded.

Body Weights: All pigs were weighed Day 0, the day of challenge (Day 28) and at necropsy. All weights were recorded.

Vaccination: At approximately 7 days of age (Day 0), ˜10 randomly allotted pigs per treatment group (Groups T01 thru T04) were vaccinated as described in Table 1. Pigs were injected in the right neck with a single dose syringe (2.0 mL intramuscular (IM) dose) of IVP, or a 2 mL IM dose of control according to allotment. A second dose of the same IVP or control was administered in the left neck at the time of weaning (˜21 days of age).

Blood Sampling: Prior to Day 0, Day 14 (prior to vaccination) and Day 28 prior to challenge (as well as Day 31, 34, 37, and 40), a blood sample was collected, using 5 mL or 9 mL serum separator tubes (dependant on body weight), from all pigs for g1TTV status (qPCR-Pfizer-VMRD Laboratory Sciences). Serum samples were aliquoted by site personnel to at least three separate tubes and were stored at −80 C.

TABLE 2
g1TTV qPCR analysis to be performed on sera by time point

| Study Day | D 0 | D 14 | D 28 | D 31 | D 34 | D 37 | D 40 |
|---|---|---|---|---|---|---|---|
| qPCR g1TTV | X | X | X | X | X | X | X |

Challenge: At ˜5 weeks of age, piglets were inoculated with a 2.0 mL (IP or IN) dose of a TTV isolate according to allotment. Challenge material was shipped to the facility identified by a treatment code for masking purposes.

Rectal Temperatures post challenge were recorded once per day on Day 28 prior to challenge as well as Day 31, 34, 37, and 40.

Necropsies: On Day 40 all animals were euthanized and necropsied. Upon necropsy, lung lesions were scored using the following methods: 1) a numeric score (0, 1, 2, 3) and 2) the percentage of consolidation for each lobe (left cranial, left middle, left caudal, right cranial, right middle, right caudal, and accessory) was scored and recorded as percent of lobe observed with lesions. Liver, kidney, thymus and lymph nodes were also scored. A blood sample was also taken prior to euthanasia. Tissues were collected as indicated in the following table:

| | | | Location of |
|---|---|---|---|
| Sample Type | Collection Method | Test | Lab |
| Inguinal, | Formalin fixed sample | Formalin fixed | Borgess |
| mesenteric | | tissue sections | Hospital |
| and bronchial | | will be | University: |
| lymph nodes | | examined for | sample |
| Thymus | Formalin fixed sample | histologic lesions. | processing |
| Spleen | Formalin fixed and sterile | Sterile | for Histology |
| | tissue samples (kidney, | Tissue samples | Pfizer Animal |
| | spleen, liver) | will be processed | Health |
| Liver | Formalin fixed and sterile | for DNA | (qPCR) |
| | tissue samples (kidney, | isolation and | |
| | spleen, liver) | quantitative | |
| Kidney | Formalin fixed and sterile | PCR analysis | |
| | tissue samples (kidney, | of g1-TTV | |
| | spleen, liver) | and g2-TTV. | |
| Formalin | Formalin fixed sample | | |
| Inflated Lung | | | |

In regard of assessment of safety and/or efficacy, no confounding secondary disease conditions were detected. Animals were vaccinated and challenged according to protocol. In regard of outcome criteria, reduction in any or all of the following were used: decreased gross or microscopic lesions; decreased viremia by qPCR; and decreased incidence of fever, weight loss or death, two-sided tests.

### Method of Analysis

Upon necropsy, lung lesions were scored using the following methods: 1) a numeric score (0=no lesions, 1=mild lesions, 2=moderate lesions, 3=severe lesions) and 2) the percentage of consolidation for each lobe (left cranial, left middle, left caudal, right cranial, right middle, right caudal, and accessory) was scored and recorded as percent of lobe observed with lesions.

The percentage of total lung with lesions was transformed and analyzed with a general linear mixed model with fixed effects, treatment, and random effect litter. Linear combinations of the parameter estimates were used in a priori contrasts after testing for a significant (P≦0.10) treatment effect. The 10% level of significance (P≦0.10) was used to assess statistical differences.

1710 qPCR data will be transformed prior to analysis with an appropriate log transformation. The transformed titers will be analyzed using a general linear repeated measures mixed model analysis. Pairwise treatment comparisons will be made at each time point if the treatment or treatment by time point interaction effect is significant (P≦0.10). Treatment least squares means, 90% confidence intervals, the minimum and maximum will be calculated and back-transformed for each time point. Descriptive statistics, means, standard deviations, and ranges, will be calculated for each treatment and day of study, pre-challenge.

### Study Results and Discussion

### Lung Lesions

Although the overall percent lung lesions observed was low throughout all treatment groups, significant differences were found. T01 (Chromos expressed g1TTV ORF1) yielded significantly lower lung lesions when compared to both the T02 (Baculovirus expressed g2TTV ORF1) and T04 (Challenge controls). Since the challenge virus was comprised of infectious g1TTV, it may not be surprising that the genotype 2 ORF1 from Baculovirus did not provide very substantially lower lung lesions as compared to the challenge controls. It is however interesting to note that while not substantial, it did offer numerically lower lung lesion scores compared to the challenge controls, thereby indicating that some level of cross protection is possible between different TTV genotypes upon optimization of dose and adjuvant selection. It was surprising that the inactivated challenge virus (T03, g1TTVp1 Killed Virus) did not offer cross-protection against the live g1TTV challenge virus as evidenced by the lack of any statistical difference between T03 and T04. This surprising lack of cross protection further enhances the veterinary importance of novel vaccines of the invention, such as g1TTV ORF1 (T01 Chromos).

| | | Back | | | | |
|---|---|---|---|---|---|---|
| | | transform | Standard | Lower 90% | Upper 90% | |
| | Number | Is mean % | error % | confidence | confidence | Range % |
| | of | lung with | lung with | limit of | limit of | lung with |
| Treatment | animals | lesions | lesions | mean | mean | lesions |
| T01 | 11 | 0.9 | 0.74 | 0.0 | 3.2 | 0 to 7.65 |
| T02 | 11 | 1.5 | 1.07 | 0.1 | 4.3 | 0 to 12.3 |
| T03 | 11 | 2.0 | 1.23 | 0.3 | 5.1 | 0.1 to 8.6 |
| T04 | 11 | 2.0 | 1.25 | 0.3 | 5.2 | 0.18 to 7.1 |

| Contrast | 2-tailed p-value (1) | significance of 2-tailed p-value |
|---|---|---|
| T01 vs T02 | 0.2167 | N.S. |
| T01 vs T02 | 0.0472 | * |
| T01 vs. T04 | 0.0389 | * |
| T02 vs T03 | 0.5394 | N.S. |
| T02 vs T04 | 0.4955 | N.S. |
| T02 vs. T04 | 0.9454 | N.S. |

(1) P-Values > 0.10 are designated as “N.S.” (Not Significant) and P-Values < or = 0.10 are designated as “*” (Signficant).

g1TTV qPCR

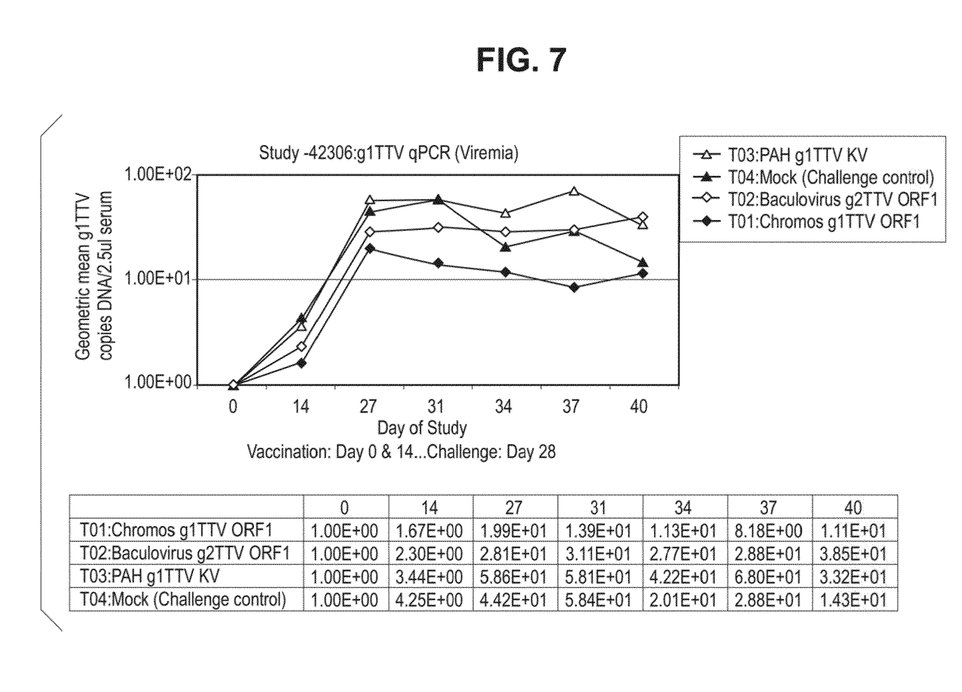

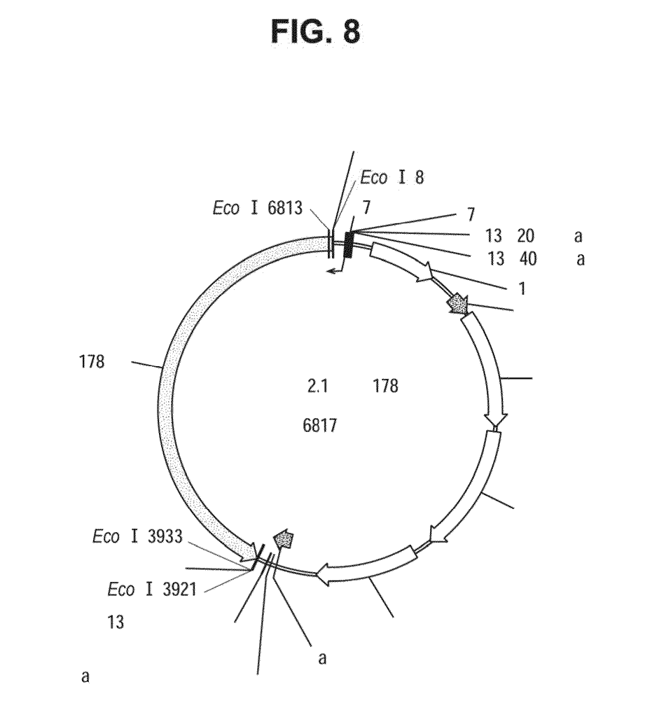

Analysis of the TTV qPCR viremia data (FIG. 7) reveals that T01 (Chromos g1TTV ORF1) has numerically lower TTV qPCR values as compared to T04 (Challenge controls). There exists a decrease in viremia magnitude and duration, which along with a reduction in lung lesions are indicators of efficacy. In addition, T02 (Baculovirus g2TTV ORF1) demonstrates a numerical reduction in viremia magnitude and duration compared to T04 (Challenge controls) but for a shorter period of time. This combined with the numerically lower lung lesions indicates that some genotypic cross protection (g2TTV ORF1 vaccine vs g1TTV challenge virus) was observed. One can suggest that with an optimized dose and adjuvant that broad genotypic cross protection can be realized. It is also interesting to note that (T03) g1TTVp1 KV offered no reduction in TTV qPCR viremia when compared to the challenge controls. This observation in conjunction with the lung lesion data further illustrate the novel finding that the recombinantly expressed g1TTV ORF1 (T01) provides efficacy as a vaccine.

### Example 4

### Codon Optimization and Recombinant Expression g1TTV ORF1 as a Full Length Protein with a 6His Tag, and Detection Thereof by an Antibody

The TTVg1 nucleotide sequence was submitted to GenScript (Piscataway, N.J., USA) for codon optimization and gene synthesis for both E. coli and Saccharomyces cerevisiae. In both cases, the codon optimized gene was cloned into the GenScript pUC57 vector, as product. The GenScript OptimumGene™ codon optimization analysis involves analysis of numerous parameters including codon usage bias, GC content, CpG dinucleotide content, mRNA secondary structure, identification of possible cryptic splicing sites, presence of premature polyA sites, internal chi sites and ribosomal binding sites, negative CpG islands, RNA instability motifs (ARE), inhibition sites (INS), repeat sequences of various kinds (including direct, reverse and dyad), and also restriction sites that may interfere with cloning. Translational performance may be additionally improved via translational initiation Kozak sequences, Shine-Dalgarno sequences, and to increase efficiency of translational termination via stop codons.

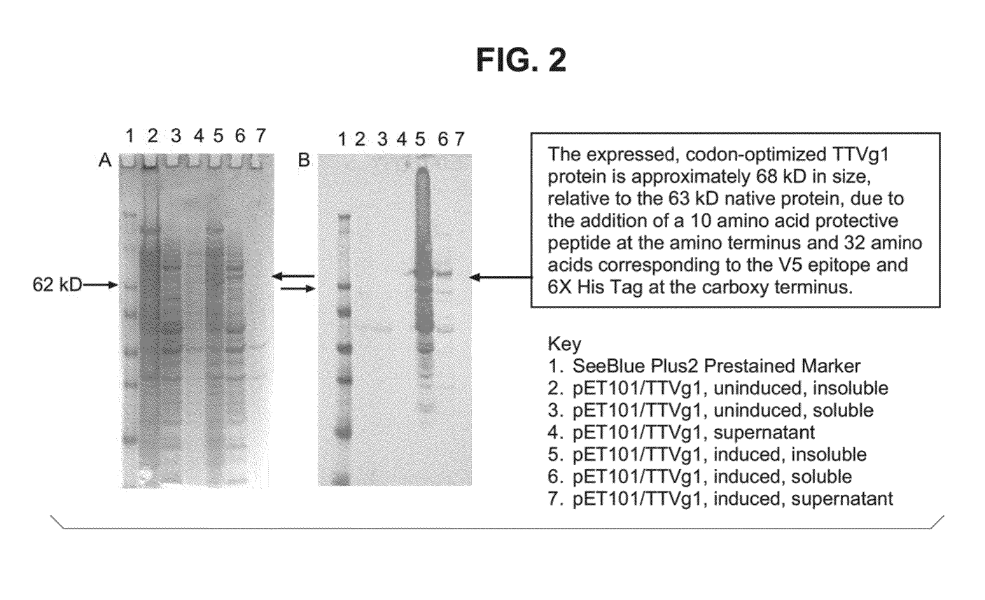

SEQ ID NO: NOS 18-20 provide TTV capsid gene that were codon optimized for both Escherichia coli (NOS: 18-19) and Saccharomyces cerevisiae (NO: 20). The sequences for E. coli are very similar, however, to clone the gene into the commercial pET101/D-TOPO expression vector (Invitrogen) to create 76057-4 (SEQ ID NO:19), additional CA nucleotides had to be added at the N-terminus. The pET101/D-TOPO expression vector also has a C-terminal V5 tag and 6X-His for purification, although the sequences for 76057-3 (SEQ ID NO:18) and 76057-4 are otherwise identical. The expressed codon-optimized TTVg1 protein is approximately kD in size, relative to the 63 kD protein, due to the addition of a 10 amino acid protective peptide at the amino terminus, and 32 amino acids corresponding to the V5 epitope and a 6X His tag at the carboxy terminus (FIG. 2).

The sequence for 76057-5 (SEQ ID NO: 20) has been codon optimized for S. cerevisiae, and it thus differs slightly from the E. coli sequences. In addition, this sequence lacks a 10 amino acid protective peptide at the N-terminus (which was added to the E. coli sequence), and it also has flanking restriction endonuclease sites, NotI at the N-terminus and AatII at the C-terminus, for subcloning of the gene into yeast vectors.

Additionally, it should be noted that the protective peptide of ten amino acids was added to N-terminus of the TTVg1 sequence for expression in E. coli. since this has been shown to increase protein stability when fused to the amino terminus. Restriction sites have been engineered such that the peptide can be removed for evaluation of the full length protein. Expression of the codon optimized TTVg1 was evaluated in the pET101/D-TOPO vector with and without the protective peptide N-terminal fusion. The TTVg1 sequence codon optimized for S. cerevisiae was also subcloned into a pESC-Trp vector with the potential for producing surface-expressed protein in yeast that can be used to elicit an antibody response in vivo.

### Example 5

### TTV Peptide Conjugation and Antibody Production (Polyclonal and Monoclonal)

Rabbit polyclonal antibodies were raised against Baculovirus expressed g2 TTV GST-ORF1 protein prepared in Example 2. Two rabbits were hyperimmunized, but only one rabbit responded. The rabbit antiserum cross-reacts to various preparations of g1 TTV whole virus that was propagated in pigs and also reacts against the immunizing antigen, Baculovirus expressed g2TTV ORF1. The rabbit antibody did not, however, respond to the E. coli expressed g2TTV ORF1 that had the 100 A.A. N-terminal arginine-rich region removed from the amino terminus as described in Example 2. This may suggest that a major antigenic epitope may be in the 100 amino acid region that was missing in the truncated g2 TTV ORF1, and that there is homology between g1 and g2 TTV in this region.

Monoclonal antibodies can be generated against full-length g1 TTV ORF1, or other g1 TTV antigens. Other potential immunizing antigens include g1 TTV whole virus, g2 TTV GST-ORF1 (Baculo), g1 TTV GST-truncated ORF1 (E. coli), and g2 TTV GST-truncated ORF1 (E. coli). A peptide library can be generated to identify linear epitopes that are antigenic. For example, 18mer peptides, with a 10 AA overlap, can be utilized to cover the TTV genome. The peptides can then be utilized in Western blots or ELISA's to determine their overall reactivity to the g1TTV ORF1 or g2TTV ORF1 monoclonal and/or polyclonal antibodies so that immunogenic domains can further be identified.

Rabbit polyclonal antibodies may also be raised against three g1 TTV ORF1 peptides cross-linked to KLH, and subsequently screened using peptide-ovalbumin conjugates. The peptide-KLH conjugates can also be used to produce monoclonal antibodies. In this respect, in one embodiment, multiple g1 TTV ORF1 peptides copies may be conjugated together, including from different strains.

In particular examples, once peptides were generated (CPC Scientific), they were then conjugated to KLH or ovalbumin (by the Proteos Co). The KLH-conjugated peptides were used for immunization of rabbits, while the Ovalbumin conjugated peptides are used for screening the serum (i.e., to detect antibodies to the peptides and not the carrier protein).

### Example 6

### Peptide Sequences for Polyclonal Antibody Generation

The following peptide sequences were chosen from TTVg1 (numbering based on AY823990) for polyclonal antibody generation, and represent SEQ ID NOS; 22-24 respectively.

1. [L167C]TTV(167-185)-NH₂:
CKDQDYWFWWDTDFKELYA-NH₂
(19 aa, pl 4)
2. TTV(459-479):
DFGHHSRFGPFCVKNEPLEFQ
(21 aa, pl 6.9)
3. [Cys612]-TTV(612-637):
CTWKRLRRMVREQLDRRMDHKRQRLH
(26 aa, pl 13)

Each of the three peptides has a single cysteine residue present in the sequence to enable selective peptide coupling to a carrier protein. In [L167C]TTV(167-185)-NH₂ and [Cys612]-TTV(612-637), an extra cysteine residue was added at the N-terminus, while in TTV(459-479) there is a native cys present at position 470. Additionally, [L167C]TTV(167-185)-NH₂ has an amidated C-terminus to yield a less acidic peptide. The peptides were selected based on sequence identity for different TTV isolates. Additionally, the C-terminal fragment [Cys612]-TTV(612-637) appears to be surface exposed. The peptides were custom made by solid phase peptide synthesis at CPC Scientific and obtained with >95% purity.

A further and highly preferred peptide is constructed by using the peptide sequence corresponding to residues 601-620 of SEQ ID NO:9 (20AA, pl 13) except that a cysteine residue is used at the N-terminus in replacement for Asn 601. This peptide is also likely surface exposed in the native protein.

### Example 7

### TTV g1 ORF1 Protein Expression Using the Chromos System

The Chromos ACE system is a protein expression platform that consists of three main components. The first component is a neutral, functional mammalian artificial chromosome called the Platform ACE, which resides in the genetic material of a modified Chinese Hamster Ovary (CHO) cell line. The second component is the ACE targeting vector, which is a plasmid used for loading target genes onto the Platform ACE. The third element is a site-specific, unidirectional integrase, which catalyzes the direct and specific loading of the target gene onto the Platform ACE. Additional information concerning the ACE System can be found of the website of Chromos Molecular Systems, Inc. of Canada, or by contacting the company directly at 604-415-7100 where the technology is available for license.

The Chromos ACE system has a number of significant advantages over traditional protein production platforms. The first of these is speed. The Chromos ACE system allows for the rapid, efficient and reproducible insertion of selected genes. The second advantage is expression. High level constitutive protein expression is achieved over time. A third advantage is stability. The Chromos ACE system allows selective and controlled protein expression. Briefly, restriction sites were added to both ends of the TTV7 ORF1 g1 DNA using PCR. Additionally, the sequence for yeast invertase was added to the 5′ end of a separate PCR preparation. The amplified sequences were then treated with restriction enzymes and sub-cloned into the plasmid pCTV927. The DNA sequence was verified by ACGT Inc. CHk2 (Chinese Hamster Ovary) cells were then transfected with the plasmids using Lipofectamine 2000 (Invitrogen), and selective pressure was added using hygromycin B. Ten single-cell clones were analyzed for TTV protein production using SDS PAGE and Western Blotting.

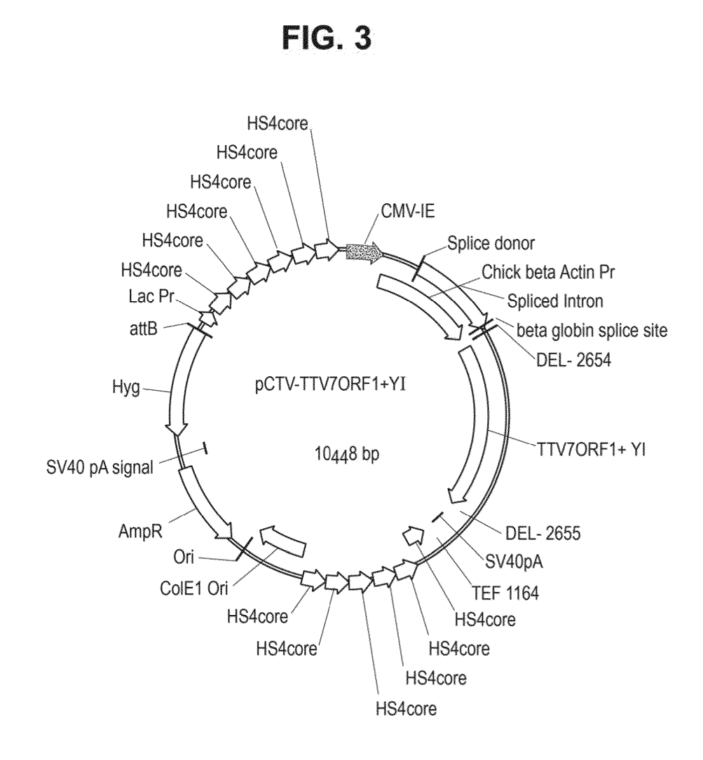

More specifically, the ACE Targeting Vector pCTV-TTV7ORF1+YI was generated as follows (see FIG. 3). The gene TTV7ORF1 was obtained as a PCR product. A primer was designed to contain the yeast invertase secretion signal and the restriction site EcoRV at the 5′ end of the gene. A second primer was designed to contain the restriction site KpnI at the 3′ end of the gene. These sequences were added to the gene TTV7ORF1 using the polymerase chain reaction. The modified gene was then subcloned into the ACE Targeting Vector ATV_{CHS4}Hyg, which contained a hygromycin resistance marker suitable for downstream antibiotic selection. The new plasmid was named pCTV-TTV7ORF1+YI.

The plasmids pCTV-TTV7ORF1+YI and pSIO343, which coded for TTV7ORF1/yeast invertase and the unidirectional lambda integrase, respectively, were transfected into the Chk2 cell line, which contained the Platform ACE. The transfected cells were named Chk2-TTV7ORF1+YI. These cells were seeded in 96-well plates and monitored for the formation of single-cell clones. Media containing Hygromycin was added to each 96-well plate to select for cell clones that contained the ACE targeting vector. Once single-cell clones were identified, twelve of them were expanded into 24-well plates, and then to 6-well plates. Finally, the clones were expanded into suspension cell culture. Culture Chk2-TTV7ORF1+YI #75 was used to generate cell-free supernatant for subsequent experimental vaccine preparation.

FIG. 7 demonstrates that Chromos-expressed g1TTV ORF1 significantly reduced lung lesions compared to the challenge controls, and reduced the numerical magitude and duration of g1TTV viremia, again compared to the challenge controls. Vaccination was at Day 0 and 14, with challenge at Day 28. The geometric mean of detected g1TTV copies was reported exponentially, i.e. 1.00 E+00 is 1, 4.25E+00 is 4.25, and 4.42E+01 is 44.2.

### Example 8

### Nuclear Localization Signals

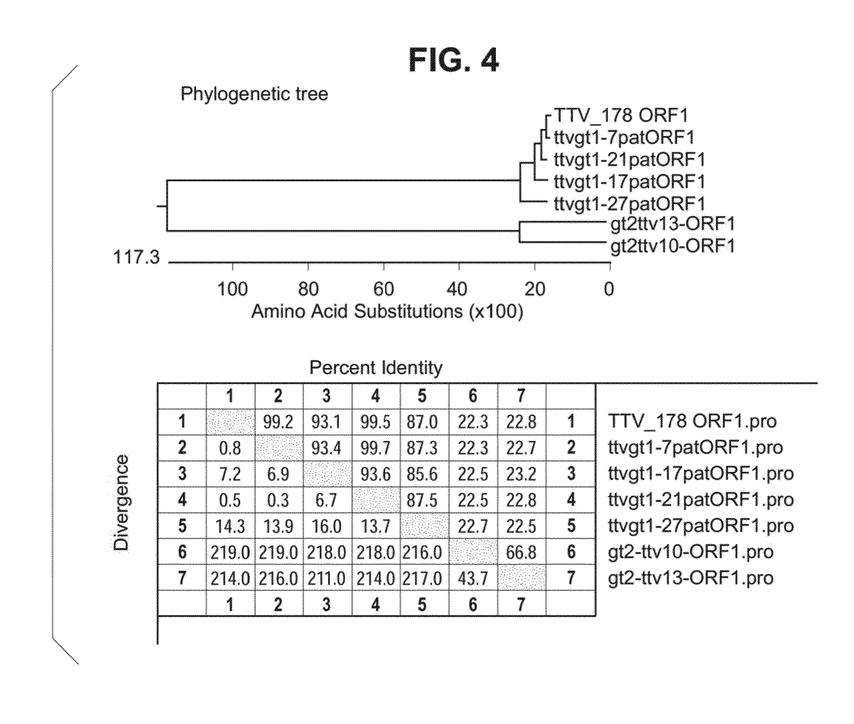

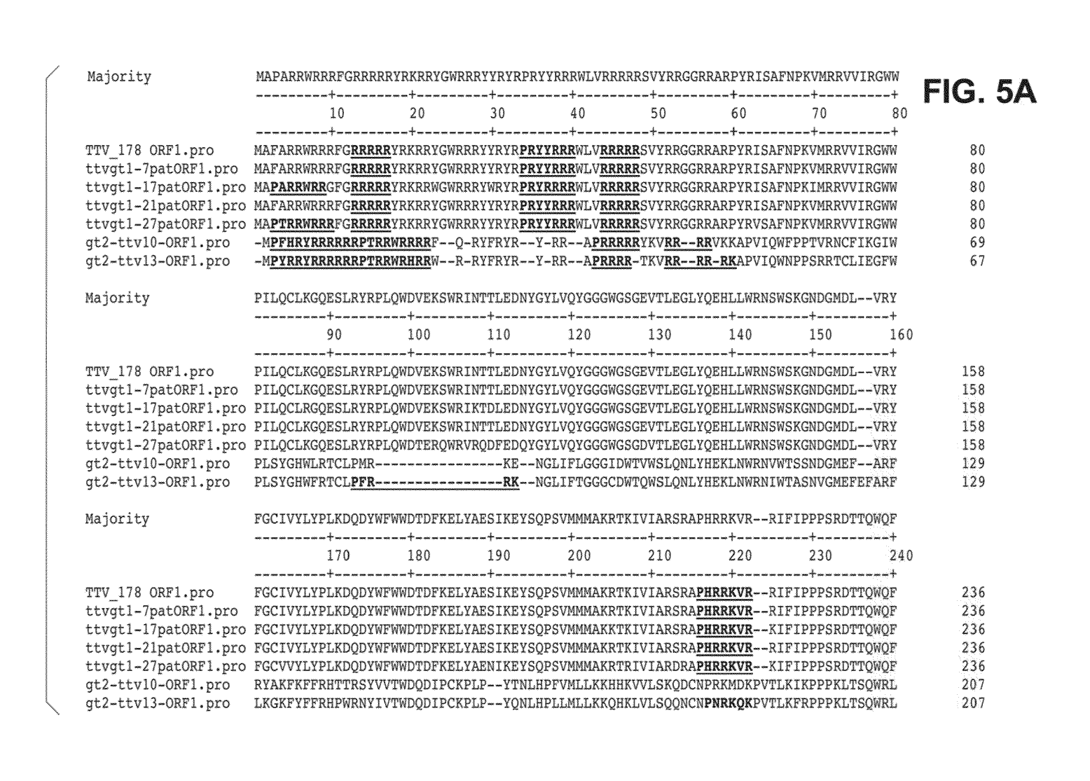

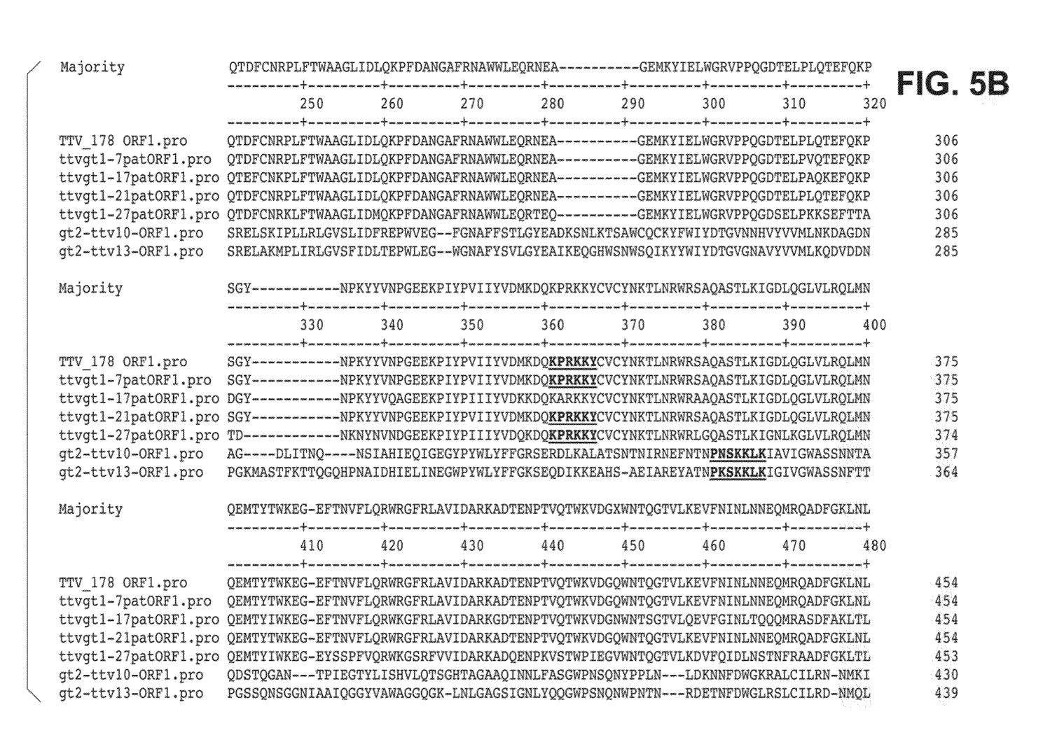

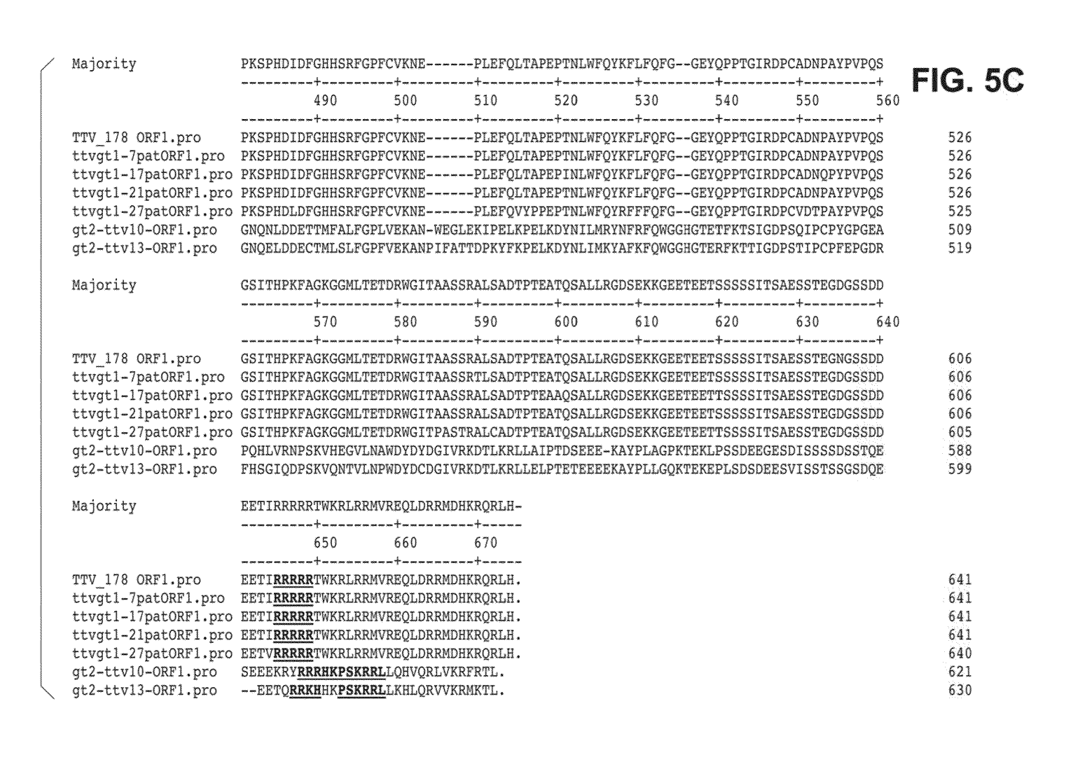

FIGS. 4 and 5 provides a 7-way amino acid alignment of ORF1 (capsid proteins) from 5 TTV gt1 viruses of the present invention and two TTV gt2 (or gt2-like) viruses of the invention. There are, of course, many gaps and mismatches because the gt1 capsids are only about 22.3 to 23.2% identical to the gt2 capsids. The five gt1 capsids are 85.6 to 99.7% identical, however, among themselves. The two gt2 capsids (TTV10 and TTV13) are similarly 66.8% identical.

Two known types of NLS signals (Pat7 and Pat4, see U.S. Pat. No. 7,544,362, for example) were identified by inspection. In FIG. 5, the NLS signals are underlined. Note the all seven capsids contain multiple NLS of both pat7 and pat4 type. Some are conserved between genotypes, some within a genotype, and some are not conserved. Most are near the N-terminus, where they tend to form overlapping poly-NLS regions. Numerous of these arginine-rich motifs are substantially immunogenic in mammals, and peptides containing them are useful in the generation of anti-TTV antibodies.

### Example 9

### Clone Fragments for Infectious Clone Construction

The following provides a basis for the construction from overlapping clones of TTV genotype 1 strain ttvgt1-178 (see SEQ ID NO:7) for which the amino acid sequence is shown as SEQ ID NO:9.

In summary, two TTV fragments (1900 bp and 2200 bp), which together span the entire TTV circular genome, were separately cloned into separate pCR 2.1 TA (Invitrogen) cloning vectors. The clone fragments were as follows: Clone 1: 680s to 2608a=˜1900 bp, and Clone 2: 1340s to 764a=˜2200 bp.

In order to accomplish this, PCR primers were designed using the consensus sequence that was generated from strains of the present invention (ttvgt1-27, -7, -17 and -21), and also from published sequences (AY823990(g1) and AB076001-(Sd-TTV31)). Primer pairs that correspond to the sequence at 680s and 2608a or 1340s and 764a were used to amplify PCR products from DNA that was extracted from liver homogenate samples of pigs infected with TTV challenge strain. These PCR fragments were cloned into Invitrogen's pCR2.1-TOPO TA vector using directions that were supplied with the kit. Clones were subsequently used to generate DNA sequences across the entire 2880 base genome and the sequence was found to be 86% homologous to published sequences GQ120664.1 and AY823990.1.

The fully correct sequences will now be combined for construction of a full length infectious clone.

### Example 10

### Infectious Clone for g1TTV

Cloning of g1TTV dsDNA Fragments.

g1TTV is a single-stranded DNA (ssDNA) virus. Fragments of g1TTV are converted to double-stranded DNA (dsDNA) using polymerase chain reaction (PCR). The dsDNA fragments of g1TTV are then cloned into pUC-based plasmid cloning vectors and transformed into E. coli. The fragments of g1TTV are less than 1 full-length dsDNA equivalent of the g1TTV genome.

Amplification of g1TTV dsDNA Concatemers.

Concatemers of full-length g1TTV dsDNA genome equivalents are generated using φ29 polymerase amplification kits (e.g., illustra TempliPhi). Full-length g1TTV dsDNA fragments are generated by digestion of the concatemers at appropriate restriction endonucleases (RE) sites. These full-length g1TTV dsDNA fragments can be cloned into plasmid vectors. Alternatively, the concatemers or the uncloned fragments (resulting from RE digestion) can be used without immediate cloning in subsequent molecular biology constructions (see below).

Tandem Duplications of the g1TTV Genome.

Plasmid constructs encoding tandem duplications of the g1TTV genome are next generated. The tandem duplications in the constructs are approximately greater than 1.2 copies of full-length dsDNA equivalents of the g1TTV genome. The tandem duplications in plasmids are generated using (1) subcloning employing appropriate RE sites, (2) PCR assembly of tandem duplications, or (3) other molecular biology methods. The templates for the generation of the tandem duplications are the g1TTV dsDNA fragments and/or the full-length g1TTV dsDNA clones (yielded by φ29 polymerase amplification).

In Vivo Recombination and Generation of g1TTV Virus.

The tandem duplication plasmid constructs are not identical to the g1TTV virus. The tandem duplication constructs are dsDNA while the virus is ssDNA, the constructs encode >1.2 full-length dsDNA equivalents of the g1TTV genome while the virus has only one full-length equivalent, the construct contains interrupting plasmid sequences while the virus has only viral sequences. To generate the bona fide g1TTV virus, the tandem duplication plasmid constructs are introduced into pigs (by inoculation, injection, electroporation, or other methods of introduction) or introduced into tissue culture cells (by transfection, electroporation, or other methods of introduction) where the plasmid construct recombines at homologous sequences to regenerate a unit-length dsDNA equivalent of the g1TTV genome. The dsDNA equivalent of the g1TTV genome is a presumed replicative intermediate of the g1TTV viral life cycle. The presence of this presumed dsDNA replicative intermediate will lead to the production of the bona fide ssDNA g1TTV.

Enabling In Vivo Generation of g1TTV Virus by Co-Transfection of g1TTV ORF-Expressing Constructs.

It is expected that a circular dsDNA g1TTV genome would be capable of yielding virus production. In the unexpected event that the dsDNA form of g1TTV is not replication-competent, the immediate expression of a g1TTV ORF may be required for the initiation of g1TTV replication from the dsDNA replicative intermediate. Plasmid constructs directing in vivo transcription of g1TTV ORFs can be made, such as the fusion of transcriptional promoters (e.g., CMV) to g1TTV ORFs. Alternatively, plasmid constructs directing the in vitro generation of g1TTV ORF transcripts can be made, such as the fusion of transcriptional promoters (e.g., T7) to g1TTV ORFs followed by use of in vitro transcription kits. Either g1TTV ORF-expressing plasmids or g1TTV ORF-expressing RNA transcripts can be co-injected into pigs or co-transfected into cells along with the tandem duplication plasmid constructs to yield g1TTV virus.

Detection of g1TTV Virus Production.

To date, whole g1TTV virus cannot be propagated in tissue culture cells. The generation of g1TTV virus is detected by immune reagents (e.g., α-g1TTV antibody) or by molecular methods (e.g., qPCR).

### Example 11

### Provision of TTV1-178 Clone in pCR2.1 Vector

Total DNA was isolated (DNEasy Blood and Tissue Kit, Qiagen, Valencia, Calif.) from a frozen liver homogenate sample (200 microliters) derived from a prior TTV challenge study. The DNA was then PCR-amplified using forward and reverse primers selected to overlap at the unique EcoRI site of the swine TTV 1-178 genome. The forward primer: for TTVg1-178 was selected as positions 1399 to 1428 (ACGG . . . CCAA) from SEQ ID NO:7, and the reverse primer: was selected to correspond to base positions 1443 (5′)-to 1416 (3′) ATAT . . . TTGT (opposite strand) from SEQ ID NO:7. PCR conditions were as follows: 1 cycle of denaturation at 94° C. for 1 minute; 35 cycles of 94° C., 30 seconds; 55° C., 30 sec; 72° C., 3 minutes; followed by a final 10 minute extension at 72° C. The resultant ˜2.8 kb fragment was cloned into pCR2.1 vector using a TOPO TA cloning kit. (Invitrogen, Carlsbad, Calif.). Upon sequence verification, this plasmid (named pCR2.1+TTV_{—}178) was found to contain the entire TTV 1-178 genome sequence.

pCR2.1+TTV_{—}178 vector was then linearized with EcoRI in order to release the full length TTV genome, which was then transfected into human embryonic kidney (293), baby hamster kidney (BHK-21), swine testicular (ST) and porcine kidney (PK) cells lines using Lipofectin (Invitrogen, Carlsbad, Calif.). The transfection was allowed to proceed for a total of 5 days at which time the cells were fixed, and then used for IFA staining to determine if the TTV DNA provided expression of ORF 1 protein. IFA staining was accomplished with rabbit polyclonal sera that was raised against a peptide corresponding to a C-terminal region of the capsid protein (residues 601-620 in SEQ ID NO:9), except that the N terminal residue thereof (Asn⁶⁰¹) was replaced with a cysteine residue. The results indicate that the TTV-transfected DNA successfully expressed the ORF-1 protein in at least 293, BHK-21, ST and PK cells lines.

### Example 12

### Preparation and Properties of Full Length Genotype 1 TTV Infectious Clone Plasmids Having Additional Sequence, Thus Providing Partial Genome Duplication

Two genotype 1 TTV infectious clone plasmids were constructed in order to propagate TTV virus in vitro (tissue culture) and/or in vivo (pigs). These plasmids contain a full length genotype 1 TTV genome with an additional partial duplication of the TTV genome (218 bp or 518 bp, see FIGS. 9 and 10) cloned into the plasmid such that the repeated sequence now flanks both ends of the non repeated TTV genomic sequence (i.e. natural 5′ end sequence from the negative strand is copied to the 3′ end also). These repeat sequences increase the chance of recombination upon transfection into mammalian cell culture or injection into pigs. When transfected into mammalian cell lines or injected into pigs, the repeat sequences recombine, resulting in removal of the additional duplicated TTV genomic sequence as well as the associated plasmid DNA. The resultant and desired product is a full length, circular TTV genome that has the ability to replicate in tissue culture and/or in the host.

TTV DNA was thus extracted from liver homogenate sample PAHg1 TTVp1 Lot#117473-178 using the UltraClean Tissue and Cells DNA Isolation Kit (Mo Bio Laboratories). The TTV was then amplified using the TTV forward primer at location 2417s (ACAAGCGACAGCGACTTCATTGACAC, SEQ ID NO:32) and reverse primer at 1639a (TATTAAGCTTCATTAGCTGTCTCAATACTA, SEQ ID NO: 33) for construction of the 218 bp duplication, or 1938a (TATTAAGCTTACACAGAAAGGTCCAAATCTACT, SEQ ID NO:34) for the 518 bp duplication. The antisense primers (1639a and 1938a) include a HindIII restriction recognition site for cloning into the pCR2.1+TTV-178 full length clone described previously. The 2417s forward primer allows for use of the unique BamHI site in the PCR amplified fragment, which resides at the position 370-376 bp in the g1 TTV genome. The ˜2100 bp (2417s/1639a) and ˜2400 bp (2417s/1938a) PCR amplified fragments are digested with BamHI/HindIII and the then ligated into the pCR2.1+TTV-178 plasmid DNA that has been digested with the same restriction endonucleases (BamHI/HindIII). All DNAs were subsequently gel purified using the Qiagen QIAquick Gel Extraction Kit and the resultant 1270 bp (218 bp repeat) and 1570 bp (518 bp repeat) sequences were ligated into the pCR2.1+TTV-178 full length backbone vector followed by transformation into the E. coli Top10 bacterial strain (Invitrogen) in order to propagate the pCR2.1+TTV-178+218OL and pCR2.1+TTV-178+518OL clones.

It will be appreciated that terminal sequence duplications of other lengths would also be effective for generation and replication of single strand circularized virus. Examples of such end sequence duplications include 100 bp, 1000 bp, 1500 bp and even duplications up to the length of the natural full length viral DNA sequence.

1. An isolated polynucleotide that comprises a full length TTV genome, wherein both ends of the genome are flanked by the same repeated nucleotide sequence, wherein recombination of the repeat sequences permits production of a circular TTV genome that replicates in tissue culture or the cells of a live host.

2. An isolated polynucleotide that comprises SEQ ID NO: 30.

3. An isolated polynucleotide that comprises SEQ ID NO:31.

4. An RNA polynucleotide molecule that is complement of any DNA polynucleotide sequence according to claim 1.

5. A vector or plasmid that comprises a polynucleotide according to claim 1.

6. A host cell that comprises a vector or plasmid according to claim 5.

7. A virus that be expressed from a polynucleotide sequence according to claim 1, wherein said virus is live, or fully or partially attenuated.

8. A vaccine comprising a virus according to claim 7.

9. A DNA vaccine that comprises a polynucleotide sequence of claim 1.


SEQUENCE LISTING (34 sequences)

SEQ ID NO: 1 - DNA, 2817 bp, Torque Teno Virus, genotype 2 gt2 TTV 10
taatgacagggttccaggaagtgctgcaaaaattacagctaaaaccacaactacttacacataaccacaaaatatttcaggaaactgcaataattttcaacacacattgcacaaaaccacaagatatcaacataaaccacaggaaactctgcaaaaaagaggaagtaaatgctattggctaaatctgaagtcttcattagcatacacaaccaaccaatcagaaacacttcctcatttgaagtatataagtaaatgcgcagacgaatggctgagtttatgccgctggtggtagacacgaacagagctgagtgtctaaccgcctgggcgggtgccggagctccagagagcggagtcaaggggcctatcgggcgggcggtaatccagcggaaccgggcccccctccatggaggagagatggctgacggtagcgtacgccgcccacggattattctgcgcctgcagtaagcccaaagaccaccttgaaaaatgcctttccaccgctatcgccgacgccgaaggagacccaccaggagatggaggagaaggaggttccagcgctactttcgatatcggtatagacgcgctcctcgccgccgccgacgctacaaggtaaggagacggagggttaaaaaggctccggtcattcaatggttccccccaacagtcagaaactgttttatcaagggaatctggccgttgagctacggacactggctccgtacctgtctccctatgagaaaagaaaacggactcatattcctaggaggtggcatagactggactgtctggagtttacagaatctataccatgaaaaactaaactggaggaatgtgtggacttcttcaaatgatggcatggagttcgctagattcagatatgcaaagtttaaattttttagacacacaaccagatcctacgtagtaacatgggaccaagacataccatgtaaacctttaccatacacaaatttacatccatttgtaatgcttctaaaaaaacatcataaagtagttctaagcaaacaagactgtaatcctagaaaaatggacaaaccagtcaccttaaaaataaagccaccaccaaaactcacatcacagtggagactaagcagagaattatcaaaaataccgctcttaagactaggagtttctttaatagacttcagagaaccatgggttgaaggttttggaaatgcattctttagtactttaggatatgaagcagataaaagcaatttaaaaacaagcgcttggtgccaatgtaaatacttctggatatatgataccggagtaaataatcatgtatatgtagtcatgttaaacaaagacgcaggagataatgcaggagacctaataacaaatcaaaactcaatagcacacatagaacagataggagaaggttatccatactggttatatttttttggaagatctgaaagagacttaaaagcactagcaacttcaaacacaaacataagaaacgaattcaatactaatcctaacagcaaaaaattaaaaatagctgtaataggatgggctagcagtaacaacacagcacaagatagtacacaaggagcgaatactccaatagaaggaacatatttaatatcacatgtgctacaaacatcaggacatacagcaggagcagcacaaataaataacctattcgcctctggatggcctaactctcaaaactatccacctttaaatctagacaaaaacaactttgactggggaaaaagagcgctatgtatactaagaaacaacatgaaaattggaaaccaaaatttagatgatgagaccactatgtttgccctcttcggacccttggtagaaaaagcaaactgggaaggcctagaaaaaataccagaactaaaaccagaactcaaagactataatatcttaatgagatataactttcgctttcagtggggcggacacggaacagagaccttcaaaacaagtattggagaccccagccaaataccctgtccctacggaccaggtgaagccccccaacaccttgtcaggaacccctccaaggtacacgagggggtcctcaatgcgtgggattatgactatgatggaattgttagaaaagacactctcaaaagactgcttgccatccccacagactcggaggaggagaaagcgtacccgctcgctggacccaaaacagagaaattgccctcctcagacgaagaaggagagagcgatatcagttcttcgagcgactcatcgacgcaagaaagcgaagaagagaagagatacagaagacgacacaagccctcaaagcgaagactcctccagcatgtccagcgactggtgaagagattcaggaccctatagacaaatacagaaacttagcagacccctcattaaatgtcacaggacattttgaacacttctgccgcttacactataaaaacatagcagaaatcagagctagaaatgccaaaaaaaacctcaataaactatacttttcagactaaaagaagtttatttctttatttaaaacaccactagagggcgtagcggggggggggacccccccgcacccccccatgcgggggcaagccccccacacccccctatgcgggggctgcgccccctgcacccccctgctaagtcacaaaatggcgggcgcggctgggacacaaaatggcggcgtagggggggggggacccccccgcaccccccctggggggggacccccctgcacccccccatgcgggggctccgccccctgcacccccgggagggggggaaaccccccctcaaccccccgcgggggggcaagcccccctgcaccccccc

SEQ ID NO: 2 - DNA, 2810 bp, Torque Teno Virus, genotype 2 gt2 TTV 13
taatgacagggttcaccggaagggctgcaaaattacagctaaaaccacaaatctaacacaataaaccacaaaatattacaggaaactgcaataaatttagaaataaattacacataaccaccaaaccacaggaaactctgcaaaaaagaggaaataaatttcattggctggtccataagtcctcattagaatacaaaaagaaccaatcagaaacacttcctcttttagagtatataagtaagtgcgcagacgaatggctgagtttatgccgctggtggtagacacgaacagagctgagtgtctaaccgcctgggcgggtgccggagctcctgagagcggagtcaaggggcctatcgggcaggcggtaatccagcggaaccgggccccccctccatggaagaaagatggctgacggtagcgtactgcgcccacggattattctgcgactgtaaagacccgaaaaaacatcttgaaaaatgccttacagacgctatcgcagacgccgaaggagaccgacaagaagatggaggcaccggaggtggagacgctactttcgatatcggtatcgacgcgctcctcgccgccgccgcacaaaggtaaggagacggaggaggaaagctccggtcatacaatggaaccctcctagccggaggacctgcctcatagaggggttctggccgttgagctacggacactggttccgtacctgtctcccctttagaagaaaaaatggactaatatttacgggaggaggttgtgactggactcagtggagcttacaaaacctttatcatgaaaaactaaactggagaaatatatggacagctagtaacgtgggaatggaattcgaattcgctagatttttaaaaggaaaattctacttttttagacatccttggagaaactatatagtgacttgggatcaggacattccttgtaaacctttaccatatcagaacttacacccattattaatgctattaaaaaaacaacacaaattagtactctcacaacaaaactgtaaccctaacagaaaacaaaaacctgtaactttaaaattcagaccgccaccaaaactaacttcacaatggagactaagtagagaattagcaaaaatgccactcattagactaggagttagttttatagacttaacagaaccgtggctagaaggttggggaaatgcattttactcagtactaggatatgaagccataaaagaacaaggacactggtcaaattggtcacaaattaaatattactggatatatgatacaggagtaggaaatgctgtatatgtagttatgctaaaacaagatgtagacgacaacccaggaaaaatggcatcaacatttaaaacaactcagggacaacatcccaatgctatagatcacatagaattaataaatgaaggatggccgtactggttatacttttttggtaaaagtgaacaagacataaaaaaggaagcacatagcgctgaaatagcaagagaatatgctacaaatccaaaatcaaaaaaactaaaaataggaatagtaggatgggcatcctctaacttcacaacaccaggcagttcacaaaactcagggggaaatatagcagcaatacaaggaggatacgtagcatgggcaggaggacaaggaaaactaaatctaggagcaggatcaataggaaatttgtaccaacaaggatggccatcaaatcaaaactggccaaatacaaacagagacgaaactaactttgattggggactcagatcactttgtatactaagagataacatgcaattaggaaatcaagaattagatgatgaatgtaccatgctctcactctttggaccttttgtagaaaaagcaaatccaatatttgcaacaacagaccctaaatactttaaaccagaactaaaagactataatttaatcatgaaatatgcctttaaattccagtggggaggacatggcacagaaagatttaaaacaaccatcggagaccccagcaccataccctgccccttcgaacccggggaccgcttccacagcgggatacaagacccctccaaggtacaaaacaccgtcctcaacccctgggactatgactgtgatgggattgttagaaaagatactctcaaaagacttctcgaactccccacagagacagaggaggaggagaaggcgtacccactccttggacaaaaaacagagaaagagccattatcagactccgacgaagagagcgttatctcaagcacgagcagtggatccgatcaagaagaagagacgcagagacgaaagcaccacaagccaagcaagcgacgactcctcaagcacctccagcgggtggtaaagaggatgaaaacactgtgatagataaatacagaaacctagcagacccctcactcaatgtcacaggacacatggaaaaattcatgcaactacatatccaaaacatacaagaaataagagctaaaaatgctaaaaaatccctcaataaactttacttttctgattaatagcggcctcctgtgtccaatctatttttttaaacacccttcaaaatggcgggagggacacaaaatggcggagggactaaggggggggcaagccccccccccaccccccatgcggggctccgccccctgcacccccacctaagtcacaaaatggcggcgcggctgggacacaaaatggcggcgtcagggggggggggaaccccccccccccctgcgggggctccgccccctgcacccccgggagggggggaaaccccccctcaaccccccgcggggggcaagcccccctgcacccccc

SEQ ID NO: 3 - DNA, 2765 bp, Torque Teno Virus, genotype 1 ttvgt1-27
tacacttccgggttcagagggctcaatttggctcgcttcgctcgcaccacgtttgctgccaggcggacctgattgaagactgaaaaccgttaagttcaaatttgaaaatggcgcccaaacatggcggaggggggcggagtttatgcaaattaatttatgcaaagtaggaggagctccattttaatttatgcaaagtaggaggagtcacttctgattggtcgggagctcaagtcctcatttgcatagggtgtaaccaatcaaacttaaggcgttcccactaaagtgaatataagtaagtgcggttccgaatggctgagtttatgccgccagcggtagacagaactgtctagcgactgggcgggtgccggaggatccctgatccggagtcaaggggcctatcgggcaggagcagctgagcggagggcctatgccggaacactgggaagaagcctggttggaagctaccaagggctggcacgacttagactgccgctgcggtaactggcaggaccacctatggctcctactcggcgatggagacgccgctttggccgccgccgtagacgctatagaaagagacgctatggctggagaagacgctactaccgctacagaccgcgttactatcggagacgatggctggtaaggagaaggcggcgttccgtctaccgtagaggtggacgtagagcgcgcccctaccgggtatctgcctttaaccccaaagtaatgcggagagtagtaataagggggtggtggccaatactacagtgcttaaaaggacaggaatcgctgagatatagaccactacagtgggacacagaaagacagtggagagtgagacaagacttcgaggatcaatacggatacctggtgcaatacggtggaggttggggaagtggtgatgtgacactagagggactataccaggaacacttactatggagaaattcctggtcaaaaggaaatgatggcatggacttagtgagatactttggctgtgtggtatacctctacccacttaaagatcaggactattggttctggtgggacactgactttaaagagctatacgcagaaaacataaaagaatacagccaaccatcagtaatgatgatggcaaaaagaactagaatagtaatagcgagagacagagctccacatagaagaaaagtgagaaaaatattcatcccaccaccatcaagagacactacgcagtggcagtttcagacagacttctgtaataggaagctatttacctgggcggcaggactaatagacatgcaaaaaccctttgatgccaacggagcttttagaaatgcgtggtggctggagcagagaacggaacagggtgaaatgaagtacatagaactgtggggaagagtgcccccacaaggagactcagaactacccaagaaaagtgaattcacaacagctacagacaataaaaactacaatgtgaatgacggtgaggaaaaacctatataccccataattatatacgtagaccaaaaagaccaaaaaccaaggaaaaagtactgtgtatgttacaacaaaactctgaacaggtggagattaggacaagcgagtactctaaaaataggaaacctgaaaggactagtgctaagacagttgatgaaccaagagatgacttacatatggaaggaaggagagtacagctcaccatttgtacaaaggtggaaaggaagcagatttgttgtgatagacgcaagaaaggctgaccaggaaaatcccaaagtatctacatggccaatagagggagtgtggaacacacagggtacagtacttaaggatgtattccagattgacttaaacagtactaatttcagagcggcagactttggaaaactaacactaccaaaatcaccgcacgacttagacttcggacatcacagtagattcggaccattctgtgtgaaaaatgaaccactggaatttcaggtatacccgccagaacccactaacctgtggtttcagtacagatttttctttcagtttggaggtgaataccaaccccccacaggaatccgcgatccatgcgttgatacaccagcctatcctgtgccgcagtcaggaagtattacacaccccaaattcgccggaaaaggcggaatgctcacggaaacagaccgttggggtatcactcctgcctctaccagagccctctgtgcagatacacccacagaagcaacgcagagtgcacttctccgaggggactcggaaaagaaaggagaggaaaccgaggaaaccacgtcatcgtccagtatcacgagtgccgaaagctctactgagggagatggatcgtctgatgatgaagagacagtcagacgccgaaggaggacctggaagcgactcagacgaatggtccgagagcagcttgaccgacgaatggaccacaagcgacagcgacttcattgacacccccattagagacagatgcctcaataaaaagcaaaagaaacgctaaactgcctccgcttattttttggggggtccggggggggcttgcccccccgaaagctgggttaccgcactaactccctgccaagtgaaactcggggacgagtgagtgcgggacatcccgtgtaatggctacataactacccggctttgcttcgacagtggccgtggctcgaccctcacacaacactgcagatagggggcgcaattgggatcgttagaaaactatggccgagcatgggcccccacaaacccccccctgcccggggctgtgccccggacccccc

SEQ ID NO: 4 - DNA, 2766 bp, Torque Teno Virus, genotype 1 ttvgt1-7
tacacttccgggttcaggaggctcaatttggctcgcttcgctcgcaccacgtttgctgccaggcggacctgtttgaagactgaaaaccgttaaattcaaatttgaaattggcggtaaacatggcggaaggggggcggagtatatgcaaattaatttatgcaaagtaggaggagctcgattttaatttatgcaaagtaggaggagtcaaatctgattggtcgggagctcaagtcctcatttgcatagggtgtaaccaatcagaattaaggcgtgcccactaaagtgaatataagtaagtgcagttccgaatggctgagtttatgccgccagcggtagacagaactgtctagcgactgggcgggtgccggaggatcccagatccggagtcaaggggcctatcgggcaggagcagctgagcggagggcctatgccggaacactgggaggaggcctggttggaagctaccaagggctggcacgaccttgactgccgctgcggtaattggcaagaccacctatggcttttgctcgccgatggagacgccgctttggccgccgccgtagacgctatagaaagagacgctatggatggaggagacgctactaccgctacagaccgcgttactatcggagacgatggctggtaaggagaaggcggcgttccgtctaccgacgaggtggacgtagagcgcgcccctaccgcatttctgcctttaatccgaaagtaatgcgtagagtagtgattagagggtggtggccaatactgcagtgcctaaaaggtcaggaatcactaagatacagaccacttcagtgggacgtagagaaaagctggagaataaacacaactcttgaggacaactatggatacttagtacagtatggaggtggttggggtagcggagaggtaacactggaggggctgtatcaggagcacctactatggagaaactcttggtcaaaaggaaacgatgggatggacttagtgagatacttcggctgcatagtatatctatatccgttaaaagatcaagactactggttttggtgggacacagattttaaagaattatatgcagagagtatcaaagaatactcacagccatctgtaatgatgatggcaaaaagaacaaaaatagtgatcgcaagaagtagagccccacatagaaggaaggtacgcagaattttcataccgcctccaagtagagacacgacacagtggcaatttcaaactgacttttgcaatagaccactattcacatgggctgcaggactcatagacctccaaaaaccatttgacgcaaacggtgcgttcagaaatgcctggtggttagaacagagaaacgaggcaggagaaatgaaatacatagagctatggggtagagtaccaccccagggggacacggaattacccgttcaaacagaattccaaaaaccctcgggatataacccaaaatactacgtaaacccgggggaggaaaaaccaatctacccagtaataatatacgtagacatgaaagaccaaaaaccaagaaaaaagtactgcgtctgctacaacaagacgcttaacaggtggcgcagcgctcaagcaagcacattaaaaattggtgacttgcaggggctagtattgagacagctaatgaaccaagaaatgacatacacatggaaagaaggagaatttaccaatgtattcctgcagaggtggagaggtttcagattagcagtaatagacgcaagaaaggcagacacagaaaacccgacagtccaaacttggaaggtggacggacagtggaacacacaagggacagtgcttaaagaggttttcaatataaacctgaataatgaacagatgagacaggcagactttggaaaactaaacttaccaaaatccccgcacgacattgactttggacaccacagtagatttggacctttctgtgtaaaaaacgaaccactggagtttcaactaacagccccagagccaactaacctgtggtttcagtacaaatttctgtttcagtttggaggtgaataccaaccaccaacaggcatccgcgatccctgcgctgataacccagcctatcctgtgccgcagtcaggaagtattacacaccccaaattcgccggaaaaggcggcatgctcacggaaacagaccgttggggtatcactgctgcctcttcccgaaccctcagtgcagatacacccacggaagcaacgcaaagtgcacttctccgaggggactcggaaaagaaaggagaggaaaccgaggaaacctcgtcatcgtccagtatcacgagtgccgaaagctctactgaaggagatggatcgtctgatgatgaagagacaatcagacgccgaaggaggacctggaagcgactcagacggatggtccgagagcagcttgaccgacgaatggaccacaagcgacagcgacttcattgacacccccattaaacagagatgcctcaataaaaaacaaaagaaacgctaagcagtgtccctattattttggggggtccggggggggcttgcccccccgtaagctgggttaccgcactaactccctgccaagtgaaactcggggacgagtgagtgcgggacatcccgtgtaatggctacataactacccggctttgcttcgacagtggccgtggctcgaccctcacacaacactgcaggtagggggcgcaattgtgatcgttagaaaactatggcccggagcatggccccccaaacccccccttgcccggggctgtgccccggaccccc

SEQ ID NO: 5 - DNA, 2768 bp, Torque Teno Virus, genotype 1 ttvgt1-17
tacacttccgggttcaggaggctcaatttggctcgcttcgctcgcaccacgtttgctgccaagcggacctgattgaagactgaaaaccgttacattcaaatttgaaaatggcgcccaaacatggcggatgtgggcggagtatatgcaaattaatttatgcaaagtaggaggagctcgattttaatttatgcaaagtaggaggagtcacttctgattggtcgggaactcaagccctcatttgcatagggtgtaaccaatcagaattaaggcgttccccgtgaagtgaatataagtaagtaaagttccgaatggctgagtttatgccgccagcggtagacagaactgtctagcgactgggcgggtgccgaaggatcccagatccggagtcaaggggcctatcgggcaggagcagctgagcggagggcctatgccggaacactgggaggaggcctggttggaagctaccaagggctggcacgacctcgactgccgctgcggtaactggcaagaccacctatggctcctgctcgccgatggagacgcggctttggccgccgccgtagacgctatagaaagagacgctggggctggagaaggcgctactggagataccgaccgcgttaccgtcggcgcagatggctggtaaggagaaggcggcgttccgtctaccgaagaggtggacgtagagcgcgcccctaccgtatttctgcttttaatccaaaaataatgcggagagtagtaataaggggatggtggccaatcctacaatgtctaagaggacaggaatcactaagatatagaccgttacagtgggacgtagaaaaaagctggagaataaagacagacttagaagacaactacggctacttagtacagtacggaggaggttgggggagcggagaggtgactctagaaggactgtaccaggaacacctactatggagaaattcatggtcaaaaggaaatgatggaatggatctagtaagatacttcggctgcatagtatacctgtacccactgaaagatcaggactactggttttggtgggacacagactttaaggaactctatgcagaaagtattaaggagtactcacaaccatcagtaatgatgatggcaaaaaaaacaaaaattgtaatagcgagaagtagggcaccacacagacgaaaagtaagaaaaatattcataccgccaccaagtagagacactacacaatggcaatttcaaacagagttctgcaacaaaccactattcacttgggctgcaggactaatagacctccaaaagccatttgacgcaaacggagcttttagaaatgcgtggtggttagaacagagaaatgaggcaggagagatgaaatacatagaattatgggggagagtcccaccgcaaggagacacagaattgccggcccaaaaagaattccagaaaccagacgggtataacccaaaatactatgtgcaggcaggagaggaaaaacctatatatccaataataatttacgtagacaaaaaagatcagaaagcaagaaagaaatactgtgtctgttacaataagacactaaacagatggagagcagcacaagcaagtaccctaaaaataggagacctgcaaggactagtactaagacaattaatgaaccaggaaatgacatatatttggaaagagggagagttcacaaacgtattcctgcaaaggtggaaaggcttcagactagcagtcatagacgccagaaagggagacacagaaaatccaacagtacaaacatggaaagtagacggaaactggaacactagtggaacagtactacaagaagtgttcggcataaacctcacccaacaacaaatgagggcatcggactttgctaagctaacactaccaaaatcgccacatgacattgactttggacaccacagtagatttgggccattttgtgtcaaaaacgaaccgctggagtttcaactaaccgctccagaacctattaatctttggtttcagtacaaatttctctttcagtttggaggtgaataccaaccaccaacaggcatccgcgatccctgcgctgataaccaaccctatcctgtgccgcagtcaggaagtattacacacccaaaattcgccgggaaaggaggaatgctcacggaaacagaccgttggggtatcactgctgcctcttccagagccctcagtgcagatacacccacggaggcagcgcaaagtgcacttctccgaggggactcggaaaagaaaggagaggaaaccgaggaaaccacgtcatcgtccagtatcacgagtgccgaaagctctactgaaggagatggatcgtctgatgatgaagagacaatccgacgcagaaggaggacctggaagcgactccgacgaatggtcagagagcagcttgaccgacgaatggaccacaagcgacagcgacttcattgacacccccataagagaacgatgcctgaataaaaaacaaaaaaaacgctacacagtgtccgcttatttgtagggggggtccggggggggcttgcccccccgtaagctgggttgccgcactaactccctgccaagtgaaactcggggacgagtgagtgcgggacatcccgtgtaatggctacataactacccggctttgcttcgacagtggccgtggctcgaccctcacacaacaatgcaggtagggggcgcaattgggatcgttagaaaactatggcccgagcatgggccccccaaaaccccccttgcccggggctgtgccccggaccccc

SEQ ID NO: 6 - DNA, 2764 bp, Torque Teno Virus, genotype 1 ttvgt1-21
tacacttccgggttcaggaggctcaatttggctcgcttcgctcgcaccacgtttgctgccaggcggacctgattgaagactgaaaaccgttaaattcaaatttgaaattggcggtaaatatggcggaaggggggcggagtatatgcaaattaatttatgcaaagtaggaggagctcgattttaatttatgcaaagtaggaggagtcaaatctgattggtcgggagctcaagtcctcatttgcatagggtgtaaccaatcagaattaaggcgtgcccactaaagtgaatataagtgagtgcagttccgaatggctgagtttatgccgccagcggtagacagaactgtctagcgactgggcgggtgccggaggatcccagatccggagtcaaggggcctatcgggcaggagcagctgagcggagggcctatgccggaacactgggaagaggcctggttggaagctaccaagggctggcacgaccttgactgccgctgcggtaattggcaagaccacctatggcttttgctcgccgatggagacgccgctttggccgccgccgtagacgctatagaaagagacgctatggatggaggagacgctactaccgctacagaccgcgttactatcggagacgatggctggtaaggagaaggcggcgttccgtctaccgacgaggtggacgtagagcgcgcccctaccgcatttctgcctttaatccgaaagtaatgcgtagagtagtgattagagggtggtggccaatactgcagtgcctaaaaggtcaggaatcactaagatacagaccacttcagtgggacgtagagaaaagctggagaataaacacaactcttgaggacaactatggatacttagtacagtatggaggtggttggggtagcggagaggtaacactggaggggctgtatcaggagcacctactatggagaaactcttggtcaaaaggaaacgatgggatggacttagtgagatacttcggctgcatagtatatctatatccgttaaaagatcaggactactggttttggtgggacacagattttaaggaattatatgcagagagtatcaaagaatactcacagccatctgtaatgatgatggcaaaaagaacaaaaatagtgatcgcaagaagtagagccccacatagaaggaaggtacgcagaattttcataccgcctccaagtagagacacgacacagtggcaatttcaaactgacttttgcaatagaccactattcacatgggctgcaggactcatagacctccaaaaaccatttgacgcaaacggtgcgttcagaaatgcctggtggttagaacagagaaacgaggcaggagaaatgaaatacatagagctatggggtagagtaccaccccagggggacacggaattaccccttcaaacagaattccaaaaaccctcgggatataacccaaaatactacgtaaacccgggggaggaaaaaccaatctacccagtaataatatacgtagacatgaaagaccaaaaaccaagaaaaaagtactgcgtctgctacaacaagacgcttaacaggtggcgcagcgctcaggcaagcacattaaaaattggtgacttgcaggggctagtattgagacagctaatgaaccaagaaatgacatacacatggaaagaaggagaatttacaaatgtattcctgcaaaggtggagaggtttcagattagcagtaatagacgctagaaaggcagacacagaaaacccgacagtccaaacttggaaggtggacggacagtggaacacacaagggacagttcttaaagaggttttcaatataaacctgaataatgaacagatgagacaggcagactttggaaaactaaacttaccaaaatccccgcacgacattgactttggacaccacagtagatttggacctttctgtgtaaaaaacgaaccactggagtttcaactaacagccccagagccaactaacctgtggtttcagtacaaatttctgtttcagtttggaggtgaataccaaccaccaacaggcatccgcgatccctgcgctgataacccagcctatcctgtgccgcagtcaggaagtattacacaccccaaattcgccggaaaaggcggcatgctcacggaaacagaccgttggggtatcactgctgcctcttcccgagccctcagtgcagatacacccacggaagcaacgcaaagtgcacttctccgaggggactcggaaaagaaaggagaggaaaccgaggaaacctcgtcatcgtccagtatcacgagtgccgaaagctctactgaaggagatggatcgtctgatgatgaagagacaatcagacgccgaaggaggacctggaagcgactcagacggatggtccgagagcagcttgaccgacgaatggaccacaagcgacagcgacttcattgacacccccattagacagagatgcctcaataaaaagcaaaagaaacgctaaacagtgtccctattactttgggggggtccggggggggcttgcccccccgtaagctgtgttaccgcactaactccctgccaagtgaaactcggggacgagtgagtgcgggacatcccgtgtaatggctacataactacccggctttgcttccacagtggccgtggctcgaccctcacacaacactgcaggtagggggcgcaattgggatcgttagaaaactatggccccaagcatggcccaaaacccccccttcccggggctgtgccccggaccccc

SEQ ID NO: 7 - DNA, 2880 bp, Torque Teno Virus, genotype 1 ttvg1-178
tacacttccgggttcaggaggctcaatttggctcgcttcgctcgcaccacgtttgctgccaggcggacvtgattgaagactgaaaaccgttaaattcaaatttgaaattggcggtaaacatggcggaaggggggcggagtatatgcaaattaatttatgcaaagtaggaggagctcgattttaatttatgcaaagtaggaggagtcaaatctgattggtcgggagcgcaagtcctcatttgcatagggtgtaaccaatcagaattaaggcgtgcccactaaagtgaatataagtaagtgcagttccgaatggctgagtttatgccgccagcggtagacagaactgtctagcgactgggcgggtgccggaggatcccagatccggagtcaaggggcctatcgggcaggagcagctgagcggagggcctatgccggaacactgggaggaggcctggttggaagctaccaagggctggcacgaccttgactgccgctgcggtaattggcaagaccacctatggcttttgctcgccgatggagacgccgctttggccgccgccgtagacgctatagaaagagacgctatggatggaggagacgctactaccgctacagaccgcgttactatcggagacgatggctggtaaggagaaggcggcgttccgtctaccgacgaggtggacgtagagcgcgcccctaccgcatttctgcctttaatccgaaagtaatgcgtagagtagtgattagagggtggtggccaatactgcagtgcctaaaaggtcaggaatcactaagatacagaccacttcagtgggacgtagagaaaagctggagaataaacacaactcttgaggacaactatggatacttagtacagtatggaggtggttggggtagcggagaggtaacactggaggggctgtatcaggagcacctactatggagaaactcttggtcaaaaggaaacgatgggatggacttagtgagatacttcggctgcatagtatatctatatccgttaaaagatcaggactactggttttggtgggacacagattttaaggaattatatgcagagagtatcaaagaatactcacagccatctgtaatgatgatggcaaaaagaacaaaaatagtgatcgcaagaagtagagccccacatagaaggaaggtacgcagaattttcataccgcctccaagtagagacacgacacagtggcaatttcaaactgacttttgcaatagaccactattcacatgggctgcaggactcatagacctccaaaaaccatttgacgcaaatggtgcgttcagaaatgcctggtggttagaacagagaaacgaggcaggagaaatgaaatacatagagctatggggtagagtaccaccccagggggacacggaattaccccttcaaacagaattccaaaaaccctcgggatataacccaaaatactacgtaaacccgggggaggaaaaaccaatctacccagtaataatatacgtagacatgaaagaccaaaaaccaagaaaaaagtactgcgtctgctacaacaagacgcttaacaggtggcgcagcgctcaggcaagcacattaaaaattggtgacttgcaggggctagtattgagacagctaatgaaccaagaaatgacatacacatggaaagaaggagaatttacaaatgtattcctgcaaaggtggagaggtttcagattagcagtaatagacgcaagaaaggcagacacagaaaacccgacagtccaaacttggaaggtggacggacagtggaacacacaaggaacagtacttaaagaggttttcaatataaacctgaataatgaacagatgagacaggcagactttggaaaactaaacttaccaaaatccccgcacgacattgactttggacaccacagtagatttggacctttctgtgtaaaaaacgaaccactggagtttcaactaacagccccagagccaactaacctgtggtttcagtacaaatttctgtttcagtttggaggtgaataccaaccaccaacaggcatccgcgatccctgcgctgataacccagcctatcctgtgccgcagtcaggaagtattacacaccccaaattcgccggaaaaggcggcatgctcacggaaacagaccgttggggtatcactgctgcctcttcccgagccctcagtgcagatacacccacggaagcaacgcaaagtgcacttctccgaggggactcggaaaagaaaggagaggaaaccgaggaaacctcgtcatcgtccagtatcacgagtgccgaaagctctactgaaggaaatggatcgtctgatgatgaagagacaatcagacgccgaaggaggacctggaagcgactcagacggatggtccgagagcagcttgaccgacgaatggaccacaagcgacagcgacttcattgacaccctccattaaagagagatgcctcaataaaaagcaaaagaaacgctaaacagtgtccctattattttgggggggcttccgggagggcttgcccccccgtaagctgggttaccgcactaactccctgccaagtgaaactcggggacgagtgagtgcgggacatcccgtgtaatggctacataactacccggctttgcttcgacagtggccgtgactcgaccctcacacaacactgcagatagggggcgcaattgggatcgttagaaaactatggccgagcatggggggggctccgccccccccaacccccccggtgggggggccaaggcccccctacacccccccatggggggctgccgccccccaaaccccccgcgtcggatggggggggctgcgccccccccaaaccccccttgcccggggctgtgccccggaccccc

SEQ ID NO: 8 - protein, 624 aa, Torque Teno Virus
  PEPTIDE (1)..(624): amino acid sequence of TTV strain AY823991 ORF1
MPYRRYRRRRRRPTRRWRHRRWRRYFRYRYRRAPRRRRTKVRRRRKKAPVIQWFPPSRRTCLIEGFWPLSYGHWFRTCLPFRRLNGLVFPGGGCDWSQWSLQNLYNEKLNWRNIWTASNVGMEFARFLKGKFYFFRHPWRNYIITWDQDIPCRPLPYQNLHPLLMLLKKQHKIVLSQQNCNPNRKQKPVTLKFKPPPKLTSQWRLSRELAKMPLIRLGVSFIDLTEPWVEGWGNAFYSVLGYEAVKDQGHWSNWTQIKYYWIYDTGVGNAVYVILLKKDVTDNPGNMATTFKASGGQHPDAIDHIELINQGWPYWLYFYGKSEQDIKKEAHSAEISREYTRDPKSKKLKIGIVGWASSNYTTTGSDQNSGGSTSAIQGGYVAYAGSGVIGAGSIGNLYQQGWPSNQNWPNTNRDKTNFDWGIRGLCILRDNMHLGSQELDDECTMLTLFGPFVEKANPIFATTDPKFFKPELKDYNIIMKYAFKFQWGGHGTERFKTNIGDPSTIPCPFEPGDRFHSGIQDPSKVQNTVLNPWDYDCDGIVRKDTLKRLLELPTETEEEEKAYPLLGQKTEKEPLSDSDEESVISSTSSGSSQEEETQRRRHHKPSKRRLLKHLQRVVKRMKTL

SEQ ID NO: 9 - protein, 640 aa, Torque Teno Virus
  PEPTIDE (1)..(640): amino acid sequence of TTV strain ttvgt1-178 ORF1 (TTV genotype 1)
MAFARRWRRRFGRRRRRYRKRRYGWRRRYYRYRPRYYRRRWLVRRRRRSVYRRGGRRARPYRISAFNPKVMRRVVIRGWWPILQCLKGQESLRYRPLQWDVEKSWRINTTLEDNYGYLVQYGGGWGSGEVTLEGLYQEHLLWRNSWSKGNDGMDLVRYFGCIVYLYPLKDQDYWFWWDTDFKELYAESIKEYSQPSVMMMAKRTKIVIARSRAPHRRKVRRIFIPPPSRDTTQWQFQTDFCNRPLFTWAAGLIDLQKPFDANGAFRNAWWLEQRNEAGEMKYIELWGRVPPQGDTELPLQTEFQKPSGYNPKYYVNPGEEKPIYPVIIYVDMKDQKPRKKYCVCYNKTLNRWRSAQASTLKIGDLQGLVLRQLMNQEMTYTWKEGEFTNVFLQRWRGFRLAVIDARKADTENPTVQTWKVDGQWNTQGTVLKEVFNINLNNEQMRQADFGKLNLPKSPHDIDFGHHSRFGPFCVKNEPLEFQLTAPEPTNLWFQYKFLFQFGGEYQPPTGIRDPCADNPAYPVPQSGSITHPKFAGKGGMLTETDRWGITAASSRALSADTPTEATQSALLRGDSEKKGEETEETSSSSSITSAESSTEGNGSSDDEETIRRRRRTWKRLRRMVREQLDRRMDHKRQRLH

SEQ ID NO: 10 - protein, 640 aa, Torque Teno Virus
  PEPTIDE (1)..(640): amino acid sequence of TTV strain ttvgt1-7 ORF1
MAFARRWRRRFGRRRRRYRKRRYGWRRRYYRYRPRYYRRRWLVRRRRRSVYRRGGRRARPYRISAFNPKVMRRVVIRGWWPILQCLKGQESLRYRPLQWDVEKSWRINTTLEDNYGYLVQYGGGWGSGEVTLEGLYQEHLLWRNSWSKGNDGMDLVRYFGCIVYLYPLKDQDYWFWWDTDFKELYAESIKEYSQPSVMMMAKRTKIVIARSRAPHRRKVRRIFIPPPSRDTTQWQFQTDFCNRPLFTWAAGLIDLQKPFDANGAFRNAWWLEQRNEAGEMKYIELWGRVPPQGDTELPVQTEFQKPSGYNPKYYVNPGEEKPIYPVIIYVDMKDQKPRKKYCVCYNKTLNRWRSAQASTLKIGDLQGLVLRQLMNQEMTYTWKEGEFTNVFLQRWRGFRLAVIDARKADTENPTVQTWKVDGQWNTQGTVLKEVFNINLNNEQMRQADFGKLNLPKSPHDIDFGHHSRFGPFCVKNEPLEFQLTAPEPTNLWFQYKFLFQFGGEYQPPTGIRDPCADNPAYPVPQSGSITHPKFAGKGGMLTETDRWGITAASSRTLSADTPTEATQSALLRGDSEKKGEETEETSSSSSITSAESSTEGDGSSDDEETIRRRRRTWKRLRRMVREQLDRRMDHKRQRLH

SEQ ID NO: 11 - protein, 640 aa, Torque Teno Virus
  PEPTIDE (1)..(640): amino acid sequence of TTV strain ttvgt1-17 ORF1
MAPARRWRRGFGRRRRRYRKRRWGWRRRYWRYRPRYRRRRWLVRRRRRSVYRRGGRRARPYRISAFNPKIMRRVVIRGWWPILQCLRGQESLRYRPLQWDVEKSWRIKTDLEDNYGYLVQYGGGWGSGEVTLEGLYQEHLLWRNSWSKGNDGMDLVRYFGCIVYLYPLKDQDYWFWWDTDFKELYAESIKEYSQPSVMMMAKKTKIVIARSRAPHRRKVRKIFIPPPSRDTTQWQFQTEFCNKPLFTWAAGLIDLQKPFDANGAFRNAWWLEQRNEAGEMKYIELWGRVPPQGDTELPAQKEFQKPDGYNPKYYVQAGEEKPIYPIIIYVDKKDQKARKKYCVCYNKTLNRWRAAQASTLKIGDLQGLVLRQLMNQEMTYIWKEGEFTNVFLQRWKGFRLAVIDARKGDTENPTVQTWKVDGNWNTSGTVLQEVFGINLTQQQMRASDFAKLTLPKSPHDIDFGHHSRFGPFCVKNEPLEFQLTAPEPINLWFQYKFLFQFGGEYQPPTGIRDPCADNQPYPVPQSGSITHPKFAGKGGMLTETDRWGITAASSRALSADTPTEAAQSALLRGDSEKKGEETEETTSSSSITSAESSTEGDGSSDDEETIRRRRRTWKRLRRMVREQLDRRMDHKRQRLH

SEQ ID NO: 12 - protein, 640 aa, Torque Teno Virus
  PEPTIDE (1)..(640): amino acid sequence of TTV strain ttvgt1-21 ORF1
MAFARRWRRRFGRRRRRYRKRRYGWRRRYYRYRPRYYRRRWLVRRRRRSVYRRGGRRARPYRISAFNPKVMRRVVIRGWWPILQCLKGQESLRYRPLQWDVEKSWRINTTLEDNYGYLVQYGGGWGSGEVTLEGLYQEHLLWRNSWSKGNDGMDLVRYFGCIVYLYPLKDQDYWFWWDTDFKELYAESIKEYSQPSVMMMAKRTKIVIARSRAPHRRKVRRIFIPPPSRDTTQWQFQTDFCNRPLFTWAAGLIDLQKPFDANGAFRNAWWLEQRNEAGEMKYIELWGRVPPQGDTELPLQTEFQKPSGYNPKYYVNPGEEKPIYPVIIYVDMKDQKPRKKYCVCYNKTLNRWRSAQASTLKIGDLQGLVLRQLMNQEMTYTWKEGEFTNVFLQRWRGFRLAVIDARKADTENPTVQTWKVDGQWNTQGTVLKEVFNINLNNEQMRQADFGKLNLPKSPHDIDFGHHSRFGPFCVKNEPLEFQLTAPEPTNLWFQYKFLFQFGGEYQPPTGIRDPCADNPAYPVPQSGSITHPKFAGKGGMLTETDRWGITAASSRALSADTPTEATQSALLRGDSEKKGEETEETSSSSSITSAESSTEGDGSSDDEETIRRRRRTWKRLRRMVREQLDRRMDHKRQRLH

SEQ ID NO: 13 - protein, 639 aa, Torque Teno Virus
  PEPTIDE (1)..(639): amino acid sequence of TTV strain ttvgt1-27 ORF1
MAPTRRWRRRFGRRRRRYRKRRYGWRRRYYRYRPRYYRRRWLVRRRRRSVYRRGGRRARPYRVSAFNPKVMRRVVIRGWWPILQCLKGQESLRYRPLQWDTERQWRVRQDFEDQYGYLVQYGGGWGSGDVTLEGLYQEHLLWRNSWSKGNDGMDLVRYFGCVVYLYPLKDQDYWFWWDTDFKELYAENIKEYSQPSVMMMAKRTRIVIARDRAPHRRKVRKIFIPPPSRDTTQWQFQTDFCNRKLFTWAAGLIDMQKPFDANGAFRNAWWLEQRTEQGEMKYIELWGRVPPQGDSELPKKSEFTTATDNKNYNVNDGEEKPIYPIIIYVDQKDQKPRKKYCVCYNKTLNRWRLGQASTLKIGNLKGLVLRQLMNQEMTYIWKEGEYSSPFVQRWKGSRFVVIDARKADQENPKVSTWPIEGVWNTQGTVLKDVFQIDLNSTNFRAADFGKLTLPKSPHDLDFGHHSRFGPFCVKNEPLEFQVYPPEPTNLWFQYRFFFQFGGEYQPPTGIRDPCVDTPAYPVPQSGSITHPKFAGKGGMLTETDRWGITPASTRALCADTPTEATQSALLRGDSEKKGEETEETTSSSSITSAESSTEGDGSSDDEETVRRRRRTWKRLRRMVREQLDRRMDHKRQRLH

SEQ ID NO: 14 - protein, 620 aa, Torque Teno Virus
  PEPTIDE (1)..(620): amino acid sequence of TTV strain gt2 TTV10 ORF1 (genotype 2)
MPFHRYRRRRRRPTRRWRRRRFQRYFRYRYRRAPRRRRRYKVRRRRVKKAPVIQWFPPTVRNCFIKGIWPLSYGHWLRTCLPMRKENGLIFLGGGIDWTVWSLQNLYHEKLNWRNVWTSSNDGMEFARFRYAKFKFFRHTTRSYVVTWDQDIPCKPLPYTNLHPFVMLLKKHHKVVLSKQDCNPRKMDKPVTLKIKPPPKLTSQWRLSRELSKIPLLRLGVSLIDFREPWVEGFGNAFFSTLGYEADKSNLKTSAWCQCKYFWIYDTGVNNHVYVVMLNKDAGDNAGDLITNQNSIAHIEQIGEGYPYWLYFFGRSERDLKALATSNTNIRNEFNTNPNSKKLKIAVIGWASSNNTAQDSTQGANTPIEGTYLISHVLQTSGHTAGAAQINNLFASGWPNSQNYPPLNLDKNNFDWGKRALCILRNNMKIGNQNLDDETTMFALFGPLVEKANWEGLEKIPELKPELKDYNILMRYNFRFQWGGHGTETFKTSIGDPSQIPCPYGPGEAPQHLVRNPSKVHEGVLNAWDYDYDGIVRKDTLKRLLAIPTDSEEEKAYPLAGPKTEKLPSSDEEGESDISSSSDSSTQESEEEKRYRRRHKPSKRRLLQHVQRLVKRFRTL

SEQ ID NO: 15 - protein, 629 aa, Torque Teno Virus
  PEPTIDE (1)..(629): amino acid sequence of TTV strain gt2 TTV13 ORF1
MPYRRYRRRRRRPTRRWRHRRWRRYFRYRYRRAPRRRRTKVRRRRRKAPVIQWNPPSRRTCLIEGFWPLSYGHWFRTCLPFRRKNGLIFTGGGCDWTQWSLQNLYHEKLNWRNIWTASNVGMEFEFARFLKGKFYFFRHPWRNYIVTWDQDIPCKPLPYQNLHPLLMLLKKQHKLVLSQQNCNPNRKQKPVTLKFRPPPKLTSQWRLSRELAKMPLIRLGVSFIDLTEPWLEGWGNAFYSVLGYEAIKEQGHWSNWSQIKYYWIYDTGVGNAVYVVMLKQDVDDNPGKMASTFKTTQGQHPNAIDHIELINEGWPYWLYFFGKSEQDIKKEAHSAEIAREYATNPKSKKLKIGIVGWASSNFTTPGSSQNSGGNIAAIQGGYVAWAGGQGKLNLGAGSIGNLYQQGWPSNQNWPNTNRDETNFDWGLRSLCILRDNMQLGNQELDDECTMLSLFGPFVEKANPIFATTDPKYFKPELKDYNLIMKYAFKFQWGGHGTERFKTTIGDPSTIPCPFEPGDRFHSGIQDPSKVQNTVLNPWDYDCDGIVRKDTLKRLLELPTETEEEEKAYPLLGQKTEKEPLSDSDEESVISSTSSGSDQEEETQRRKHHKPSKRRLLKHLQRVVKRMKTL

SEQ ID NO: 16 - DNA, 2735 bp, Torque Teno Virus, strain AY823991
  misc_feature (2596)..(2622): n is a, c, g, or t
tcatgacagggttcaccggaagggctgcaaaattacagctaaaaccacaagtctaacacaataaaccacaaagtattacaggaaactgcaataaatttagaaataagttacacataaccaccaaaccacaggaaactgtgcaaaaaagaggaaataaatttcattggctgggcctgaagtcctcattagaataataaaagaaccaatcagaagaacttcctcttttagagtatataagtaagtgcgcagacgaatggctgagtttatgccgctggtggtagacacgaacagagctgagtgtctaaccgcctgggcgggtgccggagctcctgagagcggagtcaaggggcctatcgggcaggcggtaatccagcggaaccgggcccccctcgatggaagaaagatggctgacggtagcgtactgcgcacacggattattctgcagctgtaaagacccgaaaaaacatcttgaaaaatgccttacagacgctatcgcagacgccgaagaagaccgacacggagatggaggcaccggaggtggagacgctactttcgatatcggtatcgacgcgctcctcgccgccgccgcacaaaggtaaggagacggaggaaaaaagctccggtcatacaatggttccctcctagccggagaacctgcctcatagagggattttggccgttgagctacggacactggttccgtacctgtctcccctttaggcggttaaatggactagtattcccgggtggaggttgtgactggagccagtggagtttacaaaacctttacaatgaaaaacttaactggagaaatatatggacagctagtaatgttggaatggaattcgctagatttttaaaaggaaagttttactttttcagacatccatggagaaattatataataacttgggatcaagatataccatgcaggccactaccttatcaaaacctgcatccactcctaatgctactaaaaaaacagcacaaaattgtactttcacagcaaaactgtaacccaaacagaaaacaaaaacctgtcacattaaaattcaaacctccgccaaaactaacatcacaatggagactaagtagagaattagcaaagatgccactaataagacttggagtaagctttatagacctaacagaaccatgggtagaagggtggggaaatgcattttattccgtgctaggatatgaagcagtaaaagaccaaggacactggtcaaactggacacaaataaaatactattggatctatgacacgggagtaggaaatgcagtatatgttatactattaaaaaaagacgttactgataatccaggaaacatggcaacaacctttaaagcatcaggaggacagcatccagatgcaatagatcacattgaattgataaaccaaggatggccttactggttatacttttatggtaaaagtgaacaagacattaaaaaagaggcacacagcgcagaaatatcaagagaatatactagagacccaaaatctaaaaaactaaaaataggaatagtaggatgggcatcttcaaactacacaacaacaggcagtgatcaaaacagtggtggatcaacatcagctatacaaggtggatatgtagcatatgcagggtccggggtcataggagcagggtcaataggaaatttatatcaacaaggatggccatctaatcaaaactggcctaatacaaacagagacaaaacaaactttgactggggaatacgaggactatgtatactcagagataacatgcacttaggaagccaagaattagatgatgaatgcacaatgctcacattgttcggaccctttgtagaaaaagcaaatccaatatttgcaacaacagaccctaaattctttaaacctgaactcaaagactataatataatcatgaaatatgcctttaaatttcagtggggaggacatggcacagaaagatttaaaaccaacatcggagaccccagcaccataccctgccccttcgaacccggggaccgcttccacagcgggatacaagacccctccaaggtacaaaacaccgtcctcaacccctgggactatgactgtgatgggattgttagaaaagatactctcaaaagacttctcgaactccccacagagacagaggaggaggagaaggcgtacccactccttggacaaaaaacagagaaagagccattatcagactccgacgaagagagcgttatctcaagcacgagcagtggatcctctcaagaagaagaaacgcagagacgaagacaccacaagccaagcaagcgacgactcctcaagcacctccagcgggtggtaaagaggatgaaaacactgtgatagataaatatagaaacctagcagacccctcactcaatgtcacaggacacatggaaaaattcatgcagttacatattcaaaacgtacaagaaataagagctaaaaatgctaaaaaatccctcaataaactttacttttctgattaatagcggcctcctgtgtccaacctatttttcctaaaccccttcaaaatggcgggcgggacacaaaatggcggagggactaaggggggggcaagcccccctnnnnnnnnnnnnnnnnnnnnnnnnnnnggggggcgacccccccgcacccccccctgcgggggctccgccccctgcacccccgggagggggggaaaccccccctcaaccccccgcggggggcaagcccccctgcacccccc

SEQ ID NO: 17 - DNA, 2872 bp, Torque Teno Virus, strain AY823990
  misc_feature (2719)..(2732): n is a, c, g, or t
tacactttggggttcaggaggctcaatttggctcgcttcgctcgcaccacgtttgctgccaggcggacctgattgaagactgaaaaccgttaaattcaaaattgaaaagggcgggcaaaatggcggacagggggcggagtttatgcaaattaatttatgcaaagtaggaggagctcgattttaatttatgcaaagtaggaggagtcaaatctgattggtcgggagctcaagtcctcatttgcatagggtgtaaccaatcagaattaaggcgttcccacgaaagcgaatataagtaggtgaggttccgaatggctgagtttatgccgccagcggtagacagaactgtctagcgactgggcgggtgccggaggatccctgatccggagtcaaggggcctatcgggcaggagcagctaggcggagggcctatgccggaacactgggaggaagcctggttggaagctaccaagggctggcacgatctcgactgccgctgcggtaactggcaggaccacctatggctcctactcgccgatggagacgccgctttggccgccgccgtagacgctatagaaagagacgctatggctggagacgacgctactaccgctacaggccgcgtgactatcggcgacgatggctggtaaggagaaggcggcgttccgtctaccgtagaggtggacgtagagcgcgcccctaccgactgtttaatccaaaagtaatgcggagagtagtaattagggggtggtggcctattttacaatgcttaaaaggacaggaggcactaagatatagacctctacagtgggacacagagagacagtggagagtgagatcagacttcgaagaccagtacggatacctcgtacaatacgggggaggttggggaagtggtgatgtgacacttgaaggtctctaccaagagcacttattgtggagaaactcttggtctaaaggaaacgatggaatggacctagtaagatactttggatgtgtagtatacctatatccactaaaggaccaggactattggttctggtgggacacggacttcaaagaattatatgcagaaaacataaaggaatacagccaaccatcagtaatgatgatggcaaaaagaacaagaatagtaatagccagagaaagggcaccacatagaagaaaagtaagaaaaatatttattccgccaccttcgagagacacaacacagtggcagtttcagacagatttctgcaatagaaagttatttacgtgggcagctggtctaatagacatgcaaaaaccgttcgatgctaatggagcctttagaaatgcttggtggctggaacagagaaatgatcagggagaaatgaaatacatagaactgtggggaagagtacccccacaaggagattcagagctgcccaaaaaaaaagaattctccacaggaacagataacccaaactacaatgttcaggacaatgaggagaaaaacatataccccattataatatacgtagaccaaaaagatcaaaaaccaagaaaaaagtactgcgtatgttataataagaccctcaacagatggagactaggacaggcaagtactctaaagataggaaacctgaaaggactagtactaagacagctgatgaatcaagaaatgacgtatatatggaaagaaggagaatacagtgccccctttgtacaaaggtggaaaggcagcagattcgctgtgatagacgcaagaaaggcagaccaagaaaacccgaaagtatcaacatggccaattgagggaacgtggaacacacaggacacagtactgaaggatgtattcggtattaacttgcaaaatcaacaatttagggcggcggactttggtaaactcacactaccaaaatcaccgcatgacttagacttcggtcaccacagcagatttgggccattttgtgtgaaaaatgaaccactggagtttcaggtataccctccagaaccaactaacttgtggtttcagtacagatttttctttcagtttggaggtgaataccaaccccccacaggaatccgggatccatgcgttgatacaccagcctatcctgtgccgcagtcaggaagtattacacaccccaaattcgccggaaaaggaggaatgctcacggaaacagaccgttggggtatcactgctgcctcttccagagccctcagtgcagatacacccacagaggcagcgcaaagtgcacttctccgaggggactcggaagcgaaaggagaggaaaccgaggaaaccgcgtcatcgtccagtatcacgagtgccgaaagctctactgagggagatggatcgtctgatgatgaagagacaatcagacgcagaaggaggacctggaagcgactcagacgaatggtcagagagcagcttgaccgacgaatggaccacaagcgacagcgacttcattgacacccccataagagaaagatgcctcaataaaaaacaaaagaaacgctaaacagtgtccgattactaatgggggggggtccggggggggcttgcccccccgcaagctgggttaccgcactaactccctgccaagtgaaactcggggacgagtgagtgcgggacatcccgtgtaatggctacataactacccggctttgcttcgacagtggccgtggctcgaccctcacacaacactgcaggtagggggcgcaattgggatcgttagaaaactatggccgagcatgggggnnnnnnnnnnnnnnccaacccccccggtgggggggccaaggccccccctacacccccccatggggggctgccgccccccaaaccccccgcgtcggatggggggggctgcgccccccccaaaccccccttgcccggggctgtgccccggaccccc

SEQ ID NO: 18 - DNA, 1952 bp, Torque Teno Virus
  misc_feature (1)..(1952): TTV capsid encoding sequence for strain 76057-3
ccatgggtaccaccaccaccaccacctctctgatgggtaccgcccgtcgctggcgtcgccgttttggtcgccgtcgccgtcgctatcgtaaacgtcgctacggctggcgtcgccgttattaccgttatcgcccgcgttattatcgccgtcgctggctggtgcgtcgccgtcgccgttctgtttatcgccgtggcggtcgccgtgcacgtccgtaccgtattagtgcattcaacccgaaagtgatgcgccgtgtggttattcgcggttggtggccgatcctgcagtgcctgaaaggccaagaaagtctgcgctatcgtccgctgcagtgggatgttgaaaaaagctggcgtatcaacaccacgctggaagataattatggttacctggtgcagtatggcggtggctggggtagcggcgaagttaccctggaaggcctgtaccaggaacatctgctgtggcgcaacagttggagcaagggtaatgatggcatggatctggtgcgttattttggttgtattgtttatctgtacccgctgaaagatcaggattactggttctggtgggataccgattttaaagaactgtatgcagaaagcatcaaagaatacagccagccgtctgtgatgatgatggcgaaacgcaccaaaattgtgattgcacgtagccgtgcaccgcaccgccgtaaagtgcgccgtatttttattccgccgccgtctcgcgataccacccagtggcagttccagaccgatttttgtaatcgcccgctgttcacctgggccgcaggtctgattgatctgcagaaaccgttcgatgcgaacggcgcctttcgcaatgcgtggtggctggaacagcgtaatgaagccggcgaaatgaaatatattgaactgtggggtcgtgtgccgccgcagggtgataccgaactgccggttcagacggaatttcagaaaccgagcggttacaacccgaaatattacgtgaatccgggcgaagaaaaaccgatttatccggtgatcatctacgttgatatgaaagatcagaaaccgcgcaaaaaatattgcgtgtgttacaacaaaaccctgaatcgctggcgtagtgcccaggcaagcacgctgaaaatcggtgatctgcagggcctggttctgcgtcagctgatgaaccaggaaatgacctatacgtggaaagaaggtgaatttaccaatgtgtttctgcagcgctggcgtggctttcgcctggcagttattgatgcacgtaaagcggataccgaaaacccgaccgtgcagacgtggaaagttgatggccagtggaatacccagggcacggtgctgaaagaagttttcaacatcaacctgaacaacgaacagatgcgccaggcggattttggcaaactgaacctgccgaaaagcccgcatgatatcgatttcggtcatcactctcgtttcggcccgttttgcgtgaaaaacgaaccgctggaatttcagctgaccgccccggaaccgacgaatctgtggtttcagtataaatttctgttccagtttggtggcgaataccagccgccaaccggtattcgcgatccgtgtgcggataatccggcctatccggttccgcagtctggtagtatcacccacccgaaatttgccggcaaaggtggcatgctgaccgaaacggatcgctggggcattaccgcagcgagctctcgtacgctgagcgcagataccccgacggaagcaacccagtctgcgctgctgcgtggtgatagtgagaaaaaaggcgaagaaaccgaagaaacgagtagctctagtagcattaccagcgccgaatctagtacggaaggtgatggcagctctgatgatgaagaaaccattcgccgtcgccgtcgcacctggaaacgtctgcgtcgcatggtgcgtgaacagctggatcgtcgcatggatcataaacgccagcgtctgcac

SEQ ID NO: 19 - DNA, 1954 bp, Torque Teno Virus
  misc_feature (1)..(1954): TTV capsid encoding sequence for strain 76057-4
caccatgggtaccaccaccaccaccacctctctgatgggtaccgcccgtcgctggcgtcgccgttttggtcgccgtcgccgtcgctatcgtaaacgtcgctacggctggcgtcgccgttattaccgttatcgcccgcgttattatcgccgtcgctggctggtgcgtcgccgtcgccgttctgtttatcgccgtggcggtcgccgtgcacgtccgtaccgtattagtgcattcaacccgaaagtgatgcgccgtgtggttattcgcggttggtggccgatcctgcagtgcctgaaaggccaagaaagtctgcgctatcgtccgctgcagtgggatgttgaaaaaagctggcgtatcaacaccacgctggaagataattatggttacctggtgcagtatggcggtggctggggtagcggcgaagttaccctggaaggcctgtaccaggaacatctgctgtggcgcaacagttggagcaagggtaatgatggcatggatctggtgcgttattttggttgtattgtttatctgtacccgctgaaagatcaggattactggttctggtgggataccgattttaaagaactgtatgcagaaagcatcaaagaatacagccagccgtctgtgatgatgatggcgaaacgcaccaaaattgtgattgcacgtagccgtgcaccgcaccgccgtaaagtgcgccgtatttttattccgccgccgtctcgcgataccacccagtggcagttccagaccgatttttgtaatcgcccgctgttcacctgggccgcaggtctgattgatctgcagaaaccgttcgatgcgaacggcgcctttcgcaatgcgtggtggctggaacagcgtaatgaagccggcgaaatgaaatatattgaactgtggggtcgtgtgccgccgcagggtgataccgaactgccggttcagacggaatttcagaaaccgagcggttacaacccgaaatattacgtgaatccgggcgaagaaaaaccgatttatccggtgatcatctacgttgatatgaaagatcagaaaccgcgcaaaaaatattgcgtgtgttacaacaaaaccctgaatcgctggcgtagtgcccaggcaagcacgctgaaaatcggtgatctgcagggcctggttctgcgtcagctgatgaaccaggaaatgacctatacgtggaaagaaggtgaatttaccaatgtgtttctgcagcgctggcgtggctttcgcctggcagttattgatgcacgtaaagcggataccgaaaacccgaccgtgcagacgtggaaagttgatggccagtggaatacccagggcacggtgctgaaagaagttttcaacatcaacctgaacaacgaacagatgcgccaggcggattttggcaaactgaacctgccgaaaagcccgcatgatatcgatttcggtcatcactctcgtttcggcccgttttgcgtgaaaaacgaaccgctggaatttcagctgaccgccccggaaccgacgaatctgtggtttcagtataaatttctgttccagtttggtggcgaataccagccgccaaccggtattcgcgatccgtgtgcggataatccggcctatccggttccgcagtctggtagtatcacccacccgaaatttgccggcaaaggtggcatgctgaccgaaacggatcgctggggcattaccgcagcgagctctcgtacgctgagcgcagataccccgacggaagcaacccagtctgcgctgctgcgtggtgatagtgagaaaaaaggcgaagaaaccgaagaaacgagtagctctagtagcattaccagcgccgaatctagtacggaaggtgatggcagctctgatgatgaagaaaccattcgccgtcgccgtcgcacctggaaacgtctgcgtcgcatggtgcgtgaacagctggatcgtcgcatggatcataaacgccagcgtctgcac

SEQ ID NO: 20 - DNA, 1929 bp, Torque Teno Virus
  misc_feature (1)..(1929): TTV capsid encoding sequence for strain 76057-5
ccatgggctttcgcaagaagatggagaagaagattcggtagaagaagaagaagatatagaaagagaagatacggttggagaagaagatactacagatatagaccaagatactacagaagaagatggttggttagaagaagaagaagatcagtttacagaagaggtggtagaagagctagaccttacagaatttccgcttttaatccaaaggttatgagaagagttgttattagaggttggtggcctatcttgcaatgtttgaagggtcaagaaagtttgagatacagaccattacaatgggatgttgaaaagtcttggagaattaatactacattggaagataactacggttacttagttcaatacggtggtggttggggttcaggtgaagttactttggaaggtttgtaccaagaacatttgttgtggagaaatagttggtctaagggtaacgatggtatggatttggttagatacttcggttgtatcgtttatttgtacccattgaaggatcaagattactggttctggtgggatactgatttcaaggaattgtacgctgaatctattaaggaatacagtcaaccttctgttatgatgatggcaaagagaacaaagatcgttatcgctagatcaagagcaccacatagaagaaaagttagaagaatttttattccacctccatcaagagataccactcaatggcaattccaaaccgatttttgtaatagacctttgttcacttgggctgcaggtttgattgatttgcaaaaaccattcgatgctaatggtgcttttagaaacgcttggtggttagaacaaagaaacgaagcaggtgaaatgaagtatattgaattgtggggtagagttcctccacaaggtgacactgaattgcctgttcaaacagaatttcaaaaaccttctggttacaatccaaagtattacgttaacccaggtgaagaaaagcctatctatccagttattatctatgttgatatgaaggatcaaaagccaagaaagaaatactgtgtttgttacaataagacattgaacagatggagatcagctcaagcatccaccttgaagattggtgacttgcaaggtttggttttgagacaattgatgaaccaagaaatgacatatacctggaaagaaggcgagtttactaacgttttcttgcaaagatggagaggttttagattggctgttattgatgctagaaaagcagatacagaaaatccaacagttcaaacctggaaggttgatggtcaatggaacactcaaggtacagttttgaaggaagttttcaatatcaacttaaataacgaacaaatgagacaagctgattttggtaaattgaatttgcctaagtcaccacatgatattgatttcggtcatcattccagattcggtcctttttgtgttaaaaatgaaccattggaatttcaattgacagctcctgaaccaaccaacttgtggttccaatacaagttcttgttccaattcggtggtgaataccaacctccaactggtattagagatccttgtgctgataatccagcatatcctgttccacaatcaggttccattacacatcctaaatttgctggtaaaggtggtatgttgactgaaacagatagatggggtattaccgctgcatcttcaagaactttatctgcagataccccaactgaagctacacaaagtgcattgttaagaggtgactctgaaaagaaaggtgaagaaaccgaagaaacttccagttcttcatccattacatctgctgaaagttctaccgaaggtgacggttcatccgatgatgaagaaactatcagaagaagaagaagaacatggaaaagattgagaagaatggttagagaacaattggatagaagaatggatcataagagacaaagattacatgacgtc

SEQ ID NO: 21 - DNA, 10448 bp, Torque Teno Virus
  misc_feature (1)..(10448): TTV sequence for strain ttvgt1-7, ORF1, with a yeast invertase expression tag
ggtagcgaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggttcggcggcagccaatcagaacggcgcgctccgaaagtttccttttatggcgaaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgacggtatcgataagcttgatatcgccaccatgcttttgcaagccttccttttccttttggctggttttgcagccaagatctccgcggcttttgctcgccgatggagacgccgctttggccgccgccgtagacgctatagaaagagacgctatggatggaggagacgctactaccgctacagaccgcgttactatcggagacgatggctggtaaggagaaggcggcgttccgtctaccgacgaggtggacgtagagcgcgcccctaccgcatttctgcctttaatccgaaagtaatgcgtagagtagtgattagagggtggtggccaatactgcagtgcctaaaaggtcaggaatcactaagatacagaccacttcagtgggacgtagagaaaagctggagaataaacacaactcttgaggacaactatggatacttagtacagtatggaggtggttggggtagcggagaggtaacactggaggggctgtatcaggagcacctactatggagaaactcttggtcaaaaggaaacgatgggatggacttagtgagatacttcggctgcatagtatatctatatccgttaaaagatcaagactactggttttggtgggacacagattttaaagaattatatgcagagagtatcaaagaatactcacagccatctgtaatgatgatggcaaaaagaacaaaaatagtgatcgcaagaagtagagccccacatagaaggaaggtacgcagaattttcataccgcctccaagtagagacacgacacagtggcaatttcaaactgacttttgcaatagaccactattcacatgggctgcaggactcatagacctccaaaaaccatttgacgcaaacggtgcgttcagaaatgcctggtggttagaacagagaaacgaggcaggagaaatgaaatacatagagctatggggtagagtaccaccccagggggacacggaattacccgttcaaacagaattccaaaaaccctcgggatataacccaaaatactacgtaaacccgggggaggaaaaaccaatctacccagtaataatatacgtagacatgaaagaccaaaaaccaagaaaaaagtactgcgtctgctacaacaagacgcttaacaggtggcgcagcgctcaagcaagcacattaaaaattggtgacttgcaggggctagtattgagacagctaatgaaccaagaaatgacatacacatggaaagaaggagaatttaccaatgtattcctgcagaggtggagaggtttcagattagcagtaatagacgcaagaaaggcagacacagaaaacccgacagtccaaacttggaaggtggacggacagtggaacacacaagggacagtgcttaaagaggttttcaatataaacctgaataatgaacagatgagacaggcagactttggaaaactaaacttaccaaaatccccgcacgacattgactttggacaccacagtagatttggacctttctgtgtaaaaaacgaaccactggagtttcaactaacagccccagagccaactaacctgtggtttcagtacaaatttctgtttcagtttggaggtgaataccaaccaccaacaggcatccgcgatccctgcgctgataacccagcctatcctgtgccgcagtcaggaagtattacacaccccaaattcgccggaaaaggcggcatgctcacggaaacagaccgttggggtatcactgctgcctcttcccgaaccctcagtgcagatacacccacggaagcaacgcaaagtgcacttctccgaggggactcggaaaagaaaggagaggaaaccgaggaaacctcgtcatcgtccagtatcacgagtgccgaaagctctactgaaggagatggatcgtctgatgatgaagagacaatcagacgccgaaggaggacctggaagcgactcagacggatggtccgagagcagcttgaccgacgaatggaccacaagcgacagcgacttcattgataatagggtaccgtttaaacgctagcggccgcctcaggtgcaggctgcctatcagaaggtggtggctggtgtgggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatggcaaacagctattatgggtattatgggtctcgagatctatgtcgggtgcggagaaagaggtaatgaaatggcatagggataacagggtaatactagtggatcccccgccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggccccgtatcccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcggatccgcggggctgcaggaattcgatagcttgcatgcctgcaggctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggttaattaacctggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagaactagtggatcagcgagctctagcatttaggtgacactatagaatagggccctctagcgaattctcgactcattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatgagctccgcgacgggttgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttcgcgacagacgtcgcggtgagttcaggctttttcatggatccagatttcgctcaagttagtataaaaaagcaggcttcaatcctgcagagaagctctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcctgcagccccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggccgcggatccgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggcgggggatccactagagtcgacctgcagtaactataacggtcctaa

SEQ ID NO: 22 - protein, 19 aa, Torque Teno Virus
  PEPTIDE (1)..(19): ttgvt1 peptide sequence (numbering based on the corresponding AY823990 sequence) from the ORF1 capsid protein corresponding to residues 167-185, which is used with the C-terminal AA in amidated form
CKDQDYWFWWDTDFKELYA

SEQ ID NO: 23 - protein, 21 aa, Torque Teno Virus
  PEPTIDE (1)..(21): ttgvt1 peptide sequence (numbering based on the corresponding AY823990 sequence) from the ORF1 capsid protein corresponding to residues 459-479
DFGHHSRFGPFCVKNEPLEFQ

SEQ ID NO: 24 - protein, 26 aa, Torque Teno Virus
  PEPTIDE (1)..(26): ttgvt1 peptide sequence (numbering based on the corresponding AY823990 sequence) from the ORF1 capsid protein corresponding to residues 612-637
CTWKRLRRMVREQLDRRMDHKRQRLH

SEQ ID NO: 25 - protein, 637 aa, Torque Teno Virus
  PEPTIDE (1)..(637): amino acid sequence of TTV strain AY823990 ORF1
MAPTRRWRRRFGRRRRRYRKRRYGWRRRYYRYRPRDYRRRWLVRRRRRSVYRRGGRRARPYRLFNPKVMRRVVIRGWWPILQCLKGQEALRYRPLQWDTERQWRVRSDFEDQYGYLVQYGGGWGSGDVTLEGLYQEHLLWRNSWSKGNDGMDLVRYFGCVVYLYPLKDQDYWFWWDTDFKELYAENIKEYSQPSVMMMAKRTRIVIARERAPHRRKVRKIFIPPPSRDTTQWQFQTDFCNRKLFTWAAGLIDMQKPFDANGAFRNAWWLEQRNDQGEMKYIELWGRVPPQGDSELPKKKEFSTGTDNPNYNVQDNEEKNIYPIIIYVDQKDQKPRKKYCVCYNKTLNRWRLGQASTLKIGNLKGLVLRQLMNQEMTYIWKEGEYSAPFVQRWKGSRFAVIDARKADQENPKVSTWPIEGTWNTQDTVLKDVFGINLQNQQFRAADFGKLTLPKSPHDLDFGHHSRFGPFCVKNEPLEFQVYPPEPTNLWFQYRFFFQFGGEYQPPTGIRDPCVDTPAYPVPQSGSITHPKFAGKGGMLTETDRWGITAASSRALSADTPTEAAQSALLRGDSEAKGEETEETASSSSITSAESSTEGDGSSDDEETIRRRRRTWKRLRRMVREQLDRRMDHKRQRLH

SEQ ID NO: 26 - DNA, 27 bp, Artificial Sequence
  DNA Primer
cgtactcgagtcacagtgttttcatcc

SEQ ID NO: 27 - DNA, 27 bp, Artificial Sequence
  DNA Primer
ctaggtaccatgccttacagacgctat

SEQ ID NO: 28 - DNA, 27 bp, Artificial Sequence
  DNA Primer
ctaggtaccatgcctttccaccgctat

SEQ ID NO: 29 - DNA, 26 bp, Artificial Sequence
  DNA Primer
cgtactcgagctatagggtcctgaat

SEQ ID NO: 30 - DNA, 6975 bp, Torque Teno Virus pCR2.1+TTV_178+218OL
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcattagctgtctcaatactagcccctgcaagtcaccaatttttaatgtgcttgcctgagcgctgcgccacctgttaagcgtcttgttgtagcagacgcagtacttttttcttggtttttggtctttcatgtctacgtatattattactgggtagattggtttttcctcccccgggtttacgtagtattttgggttatatcccgagggtttttggaattctgtttgaaggggtaattccgtgtccccctggggtggtactctaccccatagctctatgtatttcatttctcctgcctcgtttctctgttctaaccaccaggcatttctgaacgcaccatttgcgtcaaatggtttttggaggtctatgagtcctgcagcccatgtgaatagtggtctattgcaaaagtcagtttgaaattgccactgtgtcgtgtctctacttggaggcggtatgaaaattctgcgtaccttccttctatgtggggctctacttcttgcgatcactatttttgttctttttgccatcatcattacagatggctgtgagtattctttgatactctctgcatataattccttaaaatctgtgtcccaccaaaaccagtagtcctgatcttttaacggatatagatatactatgcagccgaagtatctcactaagtccatcccatcgtttccttttgaccaagagtttctccatagtaggtgctcctgatacagcccctccagtgttacctctccgctaccccaaccacctccatactgtactaagtatccatagttgtcctcaagagttgtgtttattctccagcttttctctacgtcccactgaagtggtctgtatcttagtgattcctgaccttttaggcmctgyagtattggccaccaccctctaatcactactctacgcattactttcggattaaaggcagaaatgcggtaggggcgcgctctacgtcmacctcgtcggtagacggaacgccgccttctccttaccagccatcgtctccgatagtaacgcggtctgtagcggtagtagcgtctcctccatccatagcgtctctttctatagcgtctacggcggcggccaaagcggcgtctccatcggcgagcaaaagccataggtggtcttgccaattaccgcagcggcagtcaaggtcgtgccagcccttggtagcttccaaccaggcctcctcccagtgttccggcataggccctccgctcagctgctcctgcccgataggccccttgactccggatctgggatcctccggcacccgcccagtcgctagacagttctgtctaccgctggcggcataaactcagccattcggaactgcacttacttatattcactttagtgggcacgccttaattctgattggttacaccctatgcaaatgaggacttgcgctcccgaccaatcagatttgactcctcctactttgcataaattaaaatcgagctcctcctactttgcataaattaatttgcatatactccgccccccttccgccatgtttaccgccaatttcaaatttgaatttaacggttttcagtcttcaatcaggtccgcctggcagcaaacgtggtgcgagcgaagcgagccaaattgagcctcctgaacccggaagtgtagggggtccggggcacagccccgggcaaggggggtttggggggggcgcagccccccccatccgacgcggggggtttggggggcggcagccccccatgggggggtgtaggggggccttggcccccccaccgggggggttggggggggcggagccccccccatgctcggccatagttttctaacgatcccaattgcgccccctatctgcagtgttgtgtgagggtcgagtcacggccactgtcgaagcaaagccgggtagttatgtagccattacacgggatgtcccgcactcactcgtccccgagtttcacttggcagggagttagtgcggtaacccagcttacgggggggcaagccctcccggaagcccccccaaaataatagggacactgtttagcgtttcttttgctttttattgaggcatctctctttaatggagggtgtcaatgaagtcgctgtcgcttgtggtccattcgtcggtcaagctgctctcggaccatccgtctgagtcgcttccaggtcctccttcggcgtctgattgtctcttcatcatcagacgatccatttccttcagtagagctttcggcactcgtgatactggacgatgacgaggtttcctcggtttcctctcctttcttttccgagtcccctcggagaagtgcactttgcgttgcttccgtgggtgtatctgcactgagggctcgggaagaggcagcagtgataccccaacggtctgtttccgtgagcatgccgccttttccggcgaatttggggtgtgtaatacttcctgactgcggcacaggataggctgggttatcagcgcagggatcgcggatgcctgttggtggttggtattcacctccaaactgaaacagaaatttgtactgaaaccacaggttagttggctctggggctgttagttgaaactccagtggttcgttttttacacagaaaggtccaaatctactgtggtgtccaaagtcaatgtcgtgcggggattttggtaagtttagttttccaaagtctgcctgtctcatctgttcattattcaggtttatattgaaaacctctttaagtactgttccttgtgtgttccactgtccgtccaccttccaagtttggactgtcgggttttctgtgtctgcctttcttgcgtctattactgctaatctgaaacctctccacctttgcaggaatacatttgtaaattctccttctttccatgtgtatgtcatttcttggttcattagctgtctcaatactagcccctgcaagtcaccaatttttaatgtgcttgcctgagcgctgcgccacctgttaagcgtcttgttgtagcagacgcagtacttttttcttggtttttggtctttcatgtctacgtatattattactgggtagattggtttttcctcccccgggtttacgtagtattttgggttatatcccgagggtttttggaattc

SEQ ID NO: 31 - DNA, 7274 bp, Torque Teno Virus pCR2.1+TTV_178+518OL
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttacacagaaaggtccaaatctactgtggtgtccaaagtcaatgtcgtgcggggattttggtaagtttagttttccaaagtctgcctgtctcatctgttcattattcaggtttatattgaaaacctctttaagtactgttccttgtgtgttccactgtccgtccaccttccaagtttggactgtcgggttttctgtgtctgcctttcttgcgtctattactgctaatctgaaacctctccacctttgcaggaatacatttgtaaattctccttctttccatgtgtatgtcatttcttggttcattagctgtctcaatactagcccctgcaagtcaccaatttttaatgtgcttgcctgagcgctgcgccacctgttaagcgtcttgttgtagcagacgcagtacttttttcttggtttttggtctttcatgtctacgtatattattactgggtagattggtttttcctcccccgggtttacgtagtattttgggttatatcccgagggtttttggaattctgtttgaaggggtaattccgtgtccccctggggtggtactctaccccatagctctatgtatttcatttctcctgcctcgtttctctgttctaaccaccaggcatttctgaacgcaccatttgcgtcaaatggtttttggaggtctatgagtcctgcagcccatgtgaatagtggtctattgcaaaagtcagtttgaaattgccactgtgtcgtgtctctacttggaggcggtatgaaaattctgcgtaccttccttctatgtggggctctacttcttgcgatcactatttttgttctttttgccatcatcattacagatggctgtgagtattctttgatactctctgcatataattccttaaaatctgtgtcccaccaaaaccagtagtcctgatcttttaacggatatagatatactatgcagccgaagtatctcactaagtccatcccatcgtttccttttgaccaagagtttctccatagtaggtgctcctgatacagcccctccagtgttacctctccgctaccccaaccacctccatactgtactaagtatccatagttgtcctcaagagttgtgtttattctccagcttttctctacgtcccactgaagtggtctgtatcttagtgattcctgaccttttaggcmctgyagtattggccaccaccctctaatcactactctacgcattactttcggattaaaggcagaaatgcggtaggggcgcgctctacgtcmacctcgtcggtagacggaacgccgccttctccttaccagccatcgtctccgatagtaacgcggtctgtagcggtagtagcgtctcctccatccatagcgtctctttctatagcgtctacggcggcggccaaagcggcgtctccatcggcgagcaaaagccataggtggtcttgccaattaccgcagcggcagtcaaggtcgtgccagcccttggtagcttccaaccaggcctcctcccagtgttccggcataggccctccgctcagctgctcctgcccgataggccccttgactccggatctgggatcctccggcacccgcccagtcgctagacagttctgtctaccgctggcggcataaactcagccattcggaactgcacttacttatattcactttagtgggcacgccttaattctgattggttacaccctatgcaaatgaggacttgcgctcccgaccaatcagatttgactcctcctactttgcataaattaaaatcgagctcctcctactttgcataaattaatttgcatatactccgccccccttccgccatgtttaccgccaatttcaaatttgaatttaacggttttcagtcttcaatcaggtccgcctggcagcaaacgtggtgcgagcgaagcgagccaaattgagcctcctgaacccggaagtgtagggggtccggggcacagccccgggcaaggggggtttggggggggcgcagccccccccatccgacgcggggggtttggggggcggcagccccccatgggggggtgtaggggggccttggcccccccaccgggggggttggggggggcggagccccccccatgctcggccatagttttctaacgatcccaattgcgccccctatctgcagtgttgtgtgagggtcgagtcacggccactgtcgaagcaaagccgggtagttatgtagccattacacgggatgtcccgcactcactcgtccccgagtttcacttggcagggagttagtgcggtaacccagcttacgggggggcaagccctcccggaagcccccccaaaataatagggacactgtttagcgtttcttttgctttttattgaggcatctctctttaatggagggtgtcaatgaagtcgctgtcgcttgtggtccattcgtcggtcaagctgctctcggaccatccgtctgagtcgcttccaggtcctccttcggcgtctgattgtctcttcatcatcagacgatccatttccttcagtagagctttcggcactcgtgatactggacgatgacgaggtttcctcggtttcctctcctttcttttccgagtcccctcggagaagtgcactttgcgttgcttccgtgggtgtatctgcactgagggctcgggaagaggcagcagtgataccccaacggtctgtttccgtgagcatgccgccttttccggcgaatttggggtgtgtaatacttcctgactgcggcacaggataggctgggttatcagcgcagggatcgcggatgcctgttggtggttggtattcacctccaaactgaaacagaaatttgtactgaaaccacaggttagttggctctggggctgttagttgaaactccagtggttcgttttttacacagaaaggtccaaatctactgtggtgtccaaagtcaatgtcgtgcggggattttggtaagtttagttttccaaagtctgcctgtctcatctgttcattattcaggtttatattgaaaacctctttaagtactgttccttgtgtgttccactgtccgtccaccttccaagtttggactgtcgggttttctgtgtctgcctttcttgcgtctattactgctaatctgaaacctctccacctttgcaggaatacatttgtaaattctccttctttccatgtgtatgtcatttcttggttcattagctgtctcaatactagcccctgcaagtcaccaatttttaatgtgcttgcctgagcgctgcgccacctgttaagcgtcttgttgtagcagacgcagtacttttttcttggtttttggtctttcatgtctacgtatattattactgggtagattggtttttcctcccccgggtttacgtagtattttgggttatatcccgagggtttttggaattc

SEQ ID NO: 32 - DNA, 26 bp, Artificial Sequence
  DNA Primer
acaagcgacagcgacttcattgacac

SEQ ID NO: 33 - DNA, 30 bp, Artificial Sequence
  DNA Primer
tattaagcttcattagctgtctcaatacta

SEQ ID NO: 34 - DNA, 33 bp, Artificial Sequence
  DNA Primer
tattaagcttacacagaaaggtccaaatctact